(19) United States
(12) Patent Application Publication
(10) Pub. No.: US 2023/0374575 A1
(43) Pub. Date: Nov. 23, 2023

(54) METHOD FOR VIRUS DETECTION

(71) Applicant: Qiagen GmbH, Hilden (DE)

(72) Inventors: Thorsten SINGER, Hilden (DE); Miroslav VRANES, Hilden (DE); Stefan CORNELIUS, Hilden (DE); Ralf PEIST, Hilden (DE)

(21) Appl. No.: 18/029,937

(22) PCT Filed: Oct. 6, 2021
(86) PCT No.: PCT/EP2021/077633

§ 371 Date: Apr. 3, 2023

Related U.S. Application Data
(60) Provisional application No. 63/088,423, filed on Oct. 6, 2020.

(30) Foreign Application Priority Data
Oct. 6, 2020 (EP) 20200425.5
Oct. 6, 2020 (EP) 20200426.3
Dec. 16, 2020 (EP) 20214412.7
Feb. 19, 2021 (EP) 21158281.2
Feb. 19, 2021 (EP) 21158283.8

(51) Int. Cl. C12Q 1/686

(52) U.S. Cl. CPC C12Q 1/686

(57) ABSTRACT

The present invention provides methods and kits for virus inactivation by virus-deactivating substances and direct amplification-based detection of target nucleic acids and viruses in biological samples without prior target purification.

### FIELD OF THE INVENTION

The present invention provides improved methods for detecting the presence or absence of a virus in a biological sample based on amplifying at least one target nucleic acid derived from the virus without prior purification of the target nucleic acid. Also provided are advantageous kits and uses.

### BACKGROUND OF THE INVENTION

Methods for detecting the presence or absence of a pathogen, such as a virus, are important and of high diagnostic value. This in particular in the situation of a pandemic where a large number of biological samples must be evaluated within a short time period in order to determine whether the subject from which the biological sample has been collected is infected with the pathogen or not. As the collected biological samples are potentially infectious, they need to be handled with great care and high safety requirements. When working with viruses for diagnostics or research it is thus always desirable to inactivate potentially comprised viruses before further processing to minimize the health risk for the operator(s) and to allow handling the biological material in a laboratory with lower biosafety requirements. This is even more important in a situation of a pandemic, such as the current COVID-19 pandemic, where extremely high throughputs of molecular diagnostic testing are required.

Therefore, the typical workflow for virus detection starts with inactivation of the virus with an aggressive chemical compound or mixture of compounds which destroy the virus due to aggressive and harsh properties. A typical example is the buffer AVL (QIAGEN; high molar guanidinium salt) as part of the QIAamp Virus Kit (QIAGEN) which is considered as a gold standard for virus inactivation and preparation. Also other chemical substances have been used and described to be suitable for virus-inactivation. It is established practice to purify the nucleic acids from the virus-inactivated sample (QIAamp Virus Kit) or extremely dilute the biological sample comprising the virus-inactivating substance in order to ensure the proper performance of the subsequently performed molecular biological analysis for detecting the presence or absence of the target virus. Most commonly used molecular biological downstream applications for detecting the presence or absence of a virus, in particular enzyme based downstream applications such as PCR assays, are sensitive to contaminating substances and can be easily inhibited. Prior purification ensures that the amplification reaction is not inhibited by contaminants that damage the used enzymes thereby allowing the detection of the target virus with high sensitivity. However, these approaches have great disadvantages. Purification of the viral nucleic acid from the biological sample requires extra steps and handling time which slows down the whole diagnostic process and requires expensive automation or a lot of hands-on time when processed manually. This is a major hurdle when high numbers of samples have to be processed as seen in the current pandemic. Dilution of the biological sample prior to detection reduces test sensitivity and may lead to false negative results especially when the virus titer is low. Accordingly, nucleic acid purification or dilution of a clinical sample to remove or dilute substances which interfere with the diagnostic downstream application for detecting the virus are time consuming, increase costs, and require special laboratory equipment, reagents and manpower which can be a limiting step as became obvious during the Covid-19 pandemic. This can therefore result in an undesired and inacceptable backlog in the processing of the samples. Consequently, there is an urgent need for improved methods.

It is thus an object of the present invention to avoid drawbacks of the prior art and to provide improved technologies for virus detection. In particular, it is an object of the present invention to provide a safe and rapid method for detecting the presence or absence of a pathogen such as a virus in a biological sample. It is furthermore an object to provide such method without compromising the detection sensitivity. It is additionally an object of the present invention to provide a rapid and safe method for detecting highly infectious viruses, such as coronaviruses, with a high sensitivity.

### SUMMARY OF THE INVENTION

The present invention overcomes core drawbacks of the prior art. In particular, the present invention provides methods and kits that provide a solution to the aforementioned problems and difficulties as is demonstrated by the examples and explained herein.

In particular, rapid methods and useful kits are provided that allow effective virus inactivation and the direct detection of target nucleic acids in biological samples, including samples contained in transport medium, without the need for prior purification of the target nucleic acid. The technology described herein is inter alia based on an improved virus inactivation using virus-deactivating substances that are suitable to inactivate viruses without impairing the sensitivity of downstream pathogen detection applications, such as amplification based detection methods. As is demonstrated by the examples, the virus inactivation technology of the present invention allows to maintain the sensitivity in direct amplification protocols that are performed using the virus-inactivated biological sample as input material. Surprisingly, and contrary to the prior art belief that virus-deactivating substances need to be removed or extremely diluted because of their chemical aggressiveness towards biological molecules, such as in particular enzymes, the present invention provides an accelerated workflow that avoids the purification or heavy dilution of the virus-inactivated biological sample prior to performing an amplification reaction. The direct amplification technology disclosed herein is rapid and avoids not only a nucleic acid purification prior to amplification, it also omits time-consuming or sample-compromising preprocessing steps such as filtration or centrifugation. Large amounts of the optionally pretreated virus-inactivated biological sample can be subjected to the amplification reaction including reverse transcription amplification reactions. The performance and/or sensitivity of the subsequent amplification reaction may be increased due to the addition of substances that counteract potential inhibitory effects of the virus-deactivating substances. The method according to the present invention is compatible with standard thermocycling and isothermal amplification procedures. Because of its rapidness and straightforward workflow, the technology of the present invention is particularly suitable for the processing of a large number of biological samples for rapid virus detection, as it is e.g. required during pandemic situations. The virus inactivation technology of the present invention ensures safe handling of potential infectious biological samples in a rapid workflow. As is shown in the present examples, the method is particularly suitable for detecting the presence or absence of RNA viruses, such as SARS-CoV-2, in respiratory samples, such as swab samples. Thus, the present invention combines effective virus inactivation using amplification compatible virus-deactivating substances with fast and reliable pathogen detection by direct amplification. The present invention thereby provides simplified and accelerated workflows that circumvent purification after inactivation and allow to apply the virus-inactivated biological sample directly in the amplification reaction for virus detection. It therefore makes an important contribution to the art.

According to a first aspect, a method for detecting the presence or absence of a virus in a biological sample based on amplifying at least one target nucleic acid derived from the virus without prior nucleic acid purification is provided, comprising
(A) providing a virus-inactivated biological sample, wherein providing such sample comprises preparing a composition comprising the biological sample and at least one virus-deactivating substance,
(B) optionally pretreating the biological sample,
(C) subjecting at least an aliquot or all of the optionally pretreated virus-inactivated biological sample to an amplification reaction and amplifying the at least one target nucleic acid, optionally wherein a reverse transcription reaction is performed in order to reverse transcribe RNA to cDNA prior to amplification.

The method according to a first aspect is particularly advantageous for providing a virus-inactivated biological sample for the direct amplification based detection of the presence of absence of a virus in a biological sample. The virus may be a RNA virus, such as in particular a coronavirus. The method allows the detection of the presence or absence of SARS-CoV-2 in a biological samples, such as respiratory specimens.

According to a second aspect, a kit is provided that is suitable for performing the method according to the first aspect and which comprises
(a) a virus-deactivating substance; and one or more and preferably all of the following components:
(b) a DNA polymerase;
(c) a reverse transcriptase;
(d) an amplification reaction buffer comprising a Mg2+ source, a buffering agent and optionally further additives;
(e) nucleotides, preferably a dNTP mix; and
(f) primers for amplifying the at least one target nucleic acid,
   optionally wherein components (b) to (e) or (b) to (f) are comprised in a single composition. Suitable virus-deactivating substances that do not compromise the amplification reaction/reverse transcription amplification reaction are disclosed herein.

The kit may further comprise
(g) an extraction composition as defined herein.

According to a third aspect, the present disclosure pertains to the use of a kit according to the second aspect in a method according to the first aspect.

According to a fourth aspect, the present disclosure pertaining to the use of a virus-deactivating substance for preparing a virus-inactivated biological sample for use in the direct amplification of target nucleic acids comprised in the biological sample without prior target nucleic acid purification, wherein preferably, the amplification reaction is a reverse transcription amplification reaction.

According to a fifth aspect the present disclosure pertains to the use of the method according to the first aspect for the detection of pathogens other than viruses.

Other objects, features, advantages and aspects of the present application will become apparent to those skilled in the art from the following description and appended claims. It should be understood, however, that the following description, appended claims, and specific examples, while indicating preferred embodiments of the application, are given by way of illustration only.

### BRIEF DESCRIPTION OF THE FIGURES

FIG. 1: Structural formulas of Lauryldimethylamin-N-oxide (LDAO) (A), Tween20 (B) and Ecosurf EH (C) (A and B are obtained from Wikipedia; C was obtained from https://www.merckroup.com/research/science-space/presentations/how-to-rescue-a-ph-sensitive-protein-detergent-viral-inactivation-and-analytical-quantitation-of-residual-detergent.pdf, retrieved on Jan. 22 2021).

FIG. 2: Ct-values of (RT-)qPCR with different amounts of LDAO, Ecosurf EH-9, Ecosurf SA-9, and Tween20 added. Black bars represent the Ct-values obtained with the IC RNA template and grey bars represent the Ct-values obtained with the DNA template. Arrows indicate the maximal non-inhibiting concentrations tested in this example.

FIG. 3: Structural formulas of povidone iodine (left) and DDAC (right).

FIG. 4: Ct-values of (RT-)qPCR with different amounts of povidone iodine or DDAC added. Black bars represent the Ct-values obtained with the IC RNA template and grey bars represent the Ct-values obtained with the DNA template. Arrows indicate the maximal non-inhibiting concentrations tested in this example.

FIG. 5: Ct-values of (RT-)qPCR for SARS-CoV-2 in negative donor samples (swab) containing heat-inactivated SARS-CoV-2 virus particles and stored for up to 10 days in 0.9% NaCl with and without 0.0145% DDAC. Grey bars indicate the detection of the N1 and N2 genes of SARS-CoV-2 and black bars indicate the detection of the IC RNA. The time points analyzed were (A) t=0, (B) day 1 (d1), (C) day 2 (d2), (D) day 6 (d6), and (E) day 10 (d10).

FIG. 6: Ct-values of (RT-)qPCR with different amounts of the non-ionic surfactants (A) Tween20, Tween60 and Brij58 as well as (B) Ecosurf EH-9 and Ecosurf SA-9.

FIG. 7: Ct-values of (RT-)PCR with increasing concentrations of SDS without and in the presence of 3% and 5% Tween20 (“Tween”; NADB, Nucleic Acid Dilution Buffer (QIAGEN, Hilden)).

FIG. 8: Ct-values of (RT-)PCR containing either didecyldimethylammonium chloride (DDAC), SDS or both (didecyldimethylammonium chloride+SDS) together in the same reaction. Black bars represent the Ct-values obtained with the IC RNA template and grey bars represent the Ct-values obtained with the DNA template.

FIG. 9: Different concentrations of the oxidizing agent povidone iodine ranging from 0.2% to 1% were mixed with increasing amounts of the reducing agent TCEP (0 mM to 5 mM). The dark (brownish) color is caused by the iodine content and disappears due to the addition of increasing concentrations of the reducing agent TCEP.

FIG. 10: Ct-values of (RT-)PCR containing povidone iodine only (0 mM TCEP) and with increasing concentrations of TECP (FAM (N1/N2): in vitro transcript N1/N2 gene SARS-CoV-2; Cy5 (IC RNA): internal control of QuantiNova Pathogen Kit; HEX (Sampling control): human DNA, simulates background of real samples).

FIG. 11: Exemplary workflows of the method according to the present invention including the use of a virus-deactivating substance and subsequent direct amplification reaction without prior nucleic acid isolation.

### DETAILED DESCRIPTION OF THE INVENTION

As explained in the summary of the invention, the different aspects and embodiments of the invention disclosed herein make important contributions to the art by providing safe and rapid workflows for pathogen detection.

### The Method According to the First Aspect

According to a first aspect, a method is provided for detecting the presence or absence of a virus in a biological sample based on amplifying at least one target nucleic acid derived from the virus without prior nucleic acid purification, comprising
(A) providing a virus-inactivated biological sample, wherein providing such sample comprises preparing a composition comprising the biological sample and at least one virus-deactivating substance,
(B) optionally pretreating the biological sample,
(C) subjecting at least an aliquot or all of the optionally pretreated virus-inactivated biological sample to an amplification reaction and amplifying the at least one target nucleic acid, optionally wherein a reverse transcription reaction is performed in order to reverse transcribe RNA to cDNA prior to amplification.

The present invention simplifies and accelerates the whole workflow by circumventing a nucleic acid purification step after virus inactivation. The virus-inactivated biological sample can be directly used in the molecular test. According to one workflow, after arrival in the lab, the collected biological sample which optionally is comprised in transport medium (e.g. UTM or VTM) is opened as usual in a—for example—biosafety laminar flow cabinet—and at least one virus-deactivating substance is added to the biological sample. Thereby, a composition comprising the biological sample and at least one virus-deactivating substance is provided which is then incubated to provide the virus-inactivated biological sample. The virus-inactivated biological sample can then be directly transferred to an analysis vessel without any risk for the operator or the amplification reagents are added to the virus—inactivated biological sample. According to another workflow, the at least one virus-deactivating substance is added directly to the transport medium (for example UTM or VTM) so that the virus is already inactivated during transport. After arrival in the lab, the virus-inactivated biological sample can be handled under less restricted conditions and can be directly contacted with the amplification reagents as described herein. The workflows may include (an) additional heating step(s) to improve lysis and/or virus inactivation.

The individual steps and preferred embodiments of the method according to the first aspect will now be described in detail.

### Step (A)

In (A) a virus-inactivated biological sample is provided, wherein providing such sample comprises preparing a composition comprising the biological sample and at least one virus-deactivating substance. Different options are available to provide the virus-inactivated biological sample in (A).

According to a preferred embodiment, providing in (A) comprises
contacting a biological sample with at least one virus-deactivating substance thereby preparing the composition, optionally wherein the biological sample is comprised in medium prior to contact with the at least one virus-deactivating substance; and
incubating the composition.

As disclosed herein, the collected biological sample (which may be comprised in medium, such as transport medium) can be contacted with the virus-deactivating substance to provide the composition in (A). Such contacting may be performed upon arrival of the collected biological sample at the site where the detection is performed.

According to a further embodiment, providing in (A) comprises
preparing the composition by immersing a collected biological sample in medium, wherein the medium comprises at least one virus-deactivating substance; and
incubating the composition.

According to this embodiment, the collected biological sample is immersed in a medium that comprises at least one virus-deactivating substance. Thereby, a composition is prepared that comprises the biological sample and at least one virus-deactivating substance. The medium for receiving the biological sample may be a transport medium that is comprised in a collection container. The collection container can be closed by a closure such as a cap. Suitable transport media and collection devices are known in the art. The composition comprising the biological sample and at least one virus-deactivating substance is incubated. During incubation the virus is inactivated thereby providing the virus-inactivated biological sample. Such incubation may occur during transportation and/or storage of the collected biological sample, respectively the collection container comprising the biological sample which is preferably contained in medium. Upon arrival at the laboratory, the virus-inactivated biological sample can be immediately handled under less restricted biosafety conditions and directly used for further analyses, such as virus detection by amplification reaction and/or reverse transcription amplification reaction, such as (RT-)PCR, without prior nucleic acid purification. This embodiment is specifically advantageous if a large number of samples needs to be analyzed in the shortest time possible and under increased safety conditions as it is the case in the situation of a pandemic.

According to one embodiment, providing in (A) comprises
immersing a biological sample in medium;
contacting the biological sample comprised in medium with at least one virus-deactivating substance thereby preparing the composition; and
incubating the composition.

According to this embodiment, the biological sample is collected and immersed in medium, e.g. a conventional transport medium. The medium may be comprised in a collection container that can be closed by a closure. Subsequently, the biological sample comprised in medium can be transported to the laboratory for further analyses, e.g. by (RT-)PCR, to detect the presence or absence of a virus. After arrival at the laboratory, the device containing the biological sample comprised in medium (e.g. collection container) is opened and the at least one virus-deactivating substance is added, thereby preparing a composition comprising the biological sample and at least one virus-deactivating substance. This initial contacting step with the virus-deactivating substance preferably occurs under specific biosafety conditions, such as under a biosafety laminar flow cabinet because at this point in time, virus-inactivation has not occurred yet. The composition comprising the biological sample and at least one virus-deactivating substance is then incubated to provide the virus-inactivated biological sample. As disclosed herein, the virus-deactivating substances according to the invention can act very rapidly. In embodiments, the incubation period for virus inactivation is ≤2 h, ≤1.5 h, ≤1 h, ≤45 min, or ≤30 min.

Incubation of the composition may be assisted by agitation.

According to one embodiment, the composition provided in (A) comprises medium that was used for collecting and/or storing the biological sample. Suitable collection/storage media are disclosed herein and are also known to the skilled person.

According to one embodiment, providing in (A) comprises heating the biological sample and/or the composition comprising the biological sample and the at least one virus-deactivating substance at a temperature that assists the virus inactivation, optionally wherein heating is performed at a temperature of ≥50° C., ≥55° C. or ≥60. In certain embodiments, heating of the biological sample and/or the composition comprising the biological sample and the at least one virus-deactivating substance is performed at a temperature of ≥75° C., ≥80° C. or ≥85° C., preferably ≥90° C. or ≥95° C. Embodiments of such heating step are also described in further detail below. Such heating step can be advantageous to assist virus-inactivation. However, the method of the invention can also be performed without such heating step. Therefore, in embodiments, virus-inactivation is achieved chemically by the use of one or more or two or more virus-deactivating substances as disclosed herein.

According to one embodiment, the virus-inactivated biological sample can be further processed within ≤2 h, ≤1 h or ≤0.5 h after contacting the biological sample with the virus-deactivating substance for the detection of the presence or absence of a virus in the biological sample by amplification reaction and/or reverse transcription amplification reaction, such as (RT-)PCR. As disclosed in the examples, virus-inactivation may occur rapidly in case a highly effective virus-deactivating substance is used. Longer incubation times may be necessary for substances with weaker virucidal activity. Preferably, the incubation time required for virus inactivation is short to accelerate the workflow. Preferably, the required incubation period is ≤1 h, ≤45 min, or ≤30 min to achieve virus inactivation. Suitable incubation times can also be determined by the skilled person.

However, the virus-inactivated biological sample may also be put on hold or stored e.g. for ≥2 h and ≤150 h, ≥3 h and ≤100 h or ≥4 h and ≤75 h, prior to further processing. In further embodiments, the storage time is at least 12 h, at least 24 h and may be at least 2 days, at least 3 days, at least 6 days or at least 10 days. The virus-inactivated biological sample may be stored at room temperature for short-term storage or at ≤4° C., ≤−20° C. or ≤−80° C. for long-term storage. As demonstrated in the examples, collecting a biological sample in medium comprising a virus-deactivating substance and subsequently performing an amplification reaction for the detection of the presence or absence of a virus without prior nucleic acid purification is possible with the method of the invention. The present invention allows to perform the virus-inactivation so that the subsequent detection is not compromised, even in case of RNA viruses. Therefore, the present invention enables flexible workflows without compromising the performance of the detection reaction. This is highly advantageous.

As disclosed herein, also two or more virus-deactivating substances may be used in combination. Hence, the composition provided in (A) may comprise two or more virus-deactivating substances. The addition of two or more virus-inactivating substances may improve virus inactivation (e.g. increased virucidal activity and/or shorter incubation for virus inactivation) thereby ensuring a safer handling of the potential infectious biological samples. The virus-deactivating substances may also be added at different points in time, e.g. at least one virus-deactivating substance may be included in the medium for collecting the biological sample and at least one virus-deactivating substance may be added prior to or during optional pretreatment of the biological sample.

The inventors observed that some very effective virus-deactivating substances that can be used in the method according to the present invention may exhibit an inhibitory effect on the amplification reaction and/or the reverse transcription, in particular when being used at higher concentrations. As is demonstrated in the examples, the present invention nevertheless allows to use such inhibitory virus-deactivating substances/concentrations, if such inhibitory effect is counteracted/reversed prior to or during performing the amplification/reverse transcription amplification. Hence, according to one embodiment, an inhibitory effect of the at least one virus-deactivating substance on the amplification reaction and/or the reverse transcription amplification is counteracted prior to or during performing the amplification reaction in (C). As shown by the examples, to achieve this, it is not required to purify the nucleic acids or heavily dilute the virus-inactivated biological sample in advance. An inhibitory effect of the at least one virus-deactivating substance may be counteracted by addition of at least one substance that can counteract the inhibitory effect of the at least one virus-deactivating substance. Advantageously, the activity of the DNA polymerase, and the reverse transcriptase (if a reverse transcription is performed), can be restored by addition of at least one substance that can counteract the inhibitory effect of the at least one virus-deactivating substance. The at least one substance that can counteract an inhibitory effect of the at least one virus-deactivating substance may be added in pretreatment step (B) and/or may be included in the amplification reaction of (C). Advantageous embodiments for suitable counteracting substances are disclosed below and also used in the examples.

The composition comprising the biological sample and the at least one virus-deactivating substance comprises the at least one virus-deactivating substance in a concentration wherein it supports or achieves virus-inactivation. Suitable concentrations can be determined by the skilled person following the detailed description and the examples presented herein. According to one embodiment, the virus-inactivated biological sample provided in (A) comprises the at least one virus-deactivating substance in a concentration where it does not interfere with the amplification reaction when at least an aliquot or all of the optionally pretreated virus-inactivated biological sample and thus the desired amount is subjected to an amplification reaction in (C). Suitable embodiments are disclosed herein and demonstrated in the examples. As the detection of RNA viruses, such as a coronavirus is of particular interest, the virus-inactivated biological sample provided in (A) preferably comprises the at least one virus-deactivating substance in a concentration where it does not interfere with the reverse transcription reaction and the amplification reaction when the desired amount of the optionally pretreated virus-inactivated biological sample is subjected to such amplification reaction in (C). As disclosed herein, also two or more virus-deactivating substances may be used in combination.

However, as disclosed herein, the one or more virus-deactivating substances may also be included in the composition and/or the amplification reaction in a concentration where they have an inhibitory effect on the amplification and/or reverse transcription reaction. In such case at least one counteracting substance may be used in a concentration so that the inhibitory effect of the virus-deactivating substance on the DNA polymerase and/or the reverse transcriptase is reduced or substantially reversed, e.g. by adding such counteracting substance during pretreatment step (B) and/or by including such substance into the amplification reaction. This allows the direct amplification and thus detection of the target nucleic acids even though the one or more virus-deactivating substances are used in an inhibiting concentration. As disclosed herein, the counteracting substance may have an inhibitory effect on its own as long as the inhibitory effects of the virus-deactivating substance and the counteracting substance compensate each other so that the resulting composition is suitable for use in a direct amplification/reverse transcription amplification. Suitable concentrations for the virus-deactivating substance and the counteracting substance to achieve such result can be determined by the skilled person by testing concentration series as it is disclosed in the examples.

To ensure a good performance in the direct amplification without prior purification, it is advantageous that the optionally pretreated virus-inactivated biological sample does not interfere with the amplification reaction when at least an aliquot or all and thus the desired amount of the optionally pretreated virus-inactivated biological sample is subjected to an amplification reaction in (C). According to one embodiment, the optionally pretreated virus-inactivated biological sample does not interfere with the reverse transcription reaction and the amplification reaction when the desired amount of the optionally pretreated virus-inactivated biological sample is subjected to an amplification reaction in (C). As discussed herein, this can be achieved by using a virus-deactivating substance in a concentration where it is effective in achieving or assisting virus-inactivation without substantial inhibition of the subsequent amplification reaction and/or reverse transcription and/or by using a counteracting substance as disclosed herein.

“Does not interfere” and similar expressions as used herein in particular means that there is no substantial inhibition of the amplification reaction so that the amplification can be carried out in a reliable manner. Furthermore, there is no substantial inhibition of the reverse transcription, if performed in advance of the amplification. This ensures that the amplification/reverse transcription amplification is performed accurately and reliably. “Does not interfere” as used herein in particular covers embodiments wherein the difference (delta) in the average Ct values obtained in (1) the amplification reaction/reverse transcription amplification reaction with the virus-inactivated biological sample (optionally pretreated) and (2) an amplification reaction with the same composition that does not comprise the virus-deactivating substance is 5 Ct values or less. For applications wherein the detection is qualitative, not quantitative, higher Ct values and thus inhibition rates in the range of up to 5 or up to 4 Ct values may be acceptable for certain users/applications. However, the present invention allows to achieve significantly better results as is demonstrated in the samples. In advantageous embodiments, the difference (delta) in the average Ct values is 3 Ct values or less, more preferably 2 Ct values or less. The average Ct value can be determined e.g. based on performing 5 to 10 identical reactions. To ensure a proper comparison, a defined amount of target nucleic acids (e.g. a control DNA or control RNA) should be added to the amplification reaction. In case a substance is additionally used in the method of the invention that counteracts an inhibitory effect of the virus-deactivating substance but has an inhibitory effect on its own, it is preferably also omitted in the reaction that omits the virus-deactivating substance to allow a proper comparison. If in such case only the virus-deactivating substance was omitted one would see inhibition effects from the counteracting substance. As is demonstrated by the examples, the technology of the invention allows efficient virus-inactivation while the average increase in the Ct values due to the virus-inactivating substance is low and can be kept in the range of <0 to 3, such as <0 to 2 or <0 to 1.5. Numerous suitable embodiments to achieve such results are described throughout the application.

### Virus-Deactivating Substances

In the method according to the invention at least one virus-deactivating substance is used for providing the virus-inactivated biological sample in (A). Also two or more virus-deactivating substances may be used in combination for virus inactivation. Virus inactivation can occur rapidly (e.g. 1.5 h or less, 1 h or less, 45 min or less or 30 min or less) if using one or more virus-deactivating substances having strong virucidal activity. According to an advantageous embodiment, the at least one virus-deactivating substance used is a disinfectant and/or a surfactant. As disclosed herein, numerous surfactants can be used in the method of the invention that are effective as disinfectants. The disinfectant preferably has a strong virucidal activity.

Preferably, a virus-deactivating substance or combination of two or more virus-deactivating substances is used in a concentration in the composition that fulfills the requirements of the established standards, such as the European Standard DIN EN 14476:2019-10 (Chemical disinfectants and antiseptics. Quantitative suspension test for the evaluation of virucidal activity in the medical area. Test method and requirements (Phase 2/Step 1), German version EN 14476:2013+A2:2019). According to this standard a substance has virucidal activity if a ≥4-log₁₀ titer reduction for virucidal suspension tests is demonstrated. The same applies if a combination of virus-deactivating substances as disclosed herein is used. As shown in the examples, oxidizing agents, such as povidone iodine, cationic surfactants, such as DDAC, as well as non-ionic surfactants, such as Ecosurf SA-7 and Ecosurf SA-9, reduced the virus titer about 4 log₁₀ demonstrating effective virucidal activity of the virus-deactivating substances according to the invention against DNA and/or RNA viruses. Preferably, the virus-deactivating substance or combination of virus-deactivating substances used for virus-inactivation in the method of the invention achieves such results against RNA and DNA viruses. Preferably, it is also active against other pathogens. Also other pathogens than viruses (e.g. bacteria, fungi) can be detected using the method according to the present invention and such use is also covered by the scope of the invention.

The virus-deactivating substance can be added in form of a liquid or solid. In embodiments, it is added in form of a solution that comprises or consists of the virus-deactivating substance. Concentrations suitable to achieve virus-inactivation while not interfering with the downstream direct amplification can be determined by the skilled person following the guidance provided herein and the examples. As disclosed therein, concentration series can be performed to determine a concentration for the virus-deactivating substance that achieves the desired degree of virus-inactivation in the biological sample while ensuring a good performance of the virus-inactivated biological sample in the amplification/reverse transcription reaction (optionally in combination with a counteracting substance as disclosed herein). While efficient virus-inactivation must be ensured, it is advantageous to keep the amount of added virus-inactivating substance low to avoid an unnecessary dilution of the biological sample and/or inhibition of the subsequent amplification. In preferred embodiments, the biological sample (optionally comprised in medium, such as transport medium) provides at least 70%, at least 80%, at least 90%, at least 95% or at least 98% of the composition that comprises the biological sample (optionally comprised in medium) and the virus-deactivating substance. The present invention provides numerous virus-deactivating substances that achieve virus inactivation already in low concentrations and do not interfere with the subsequent amplification/reverse transcription amplification reaction.

### Disinfectants Such as Oxidizing Agents

According to one embodiment, the virus-deactivating substance is an oxidizing agent. As is demonstrated in the examples using povidone iodine, oxidizing agents can be advantageously used as virus-deactivating substance. The present invention allows to use oxidizing agents having a strong virucidal activity that do not compromise the performance of the subsequently performed direct amplification reaction. Furthermore, a reducing agent may be added as counteracting substance to compensate an inhibitory effect of the oxidizing agent on the DNA polymerase and/or reverse transcriptase, if necessary.

Suitable disinfectants, such as oxidizing agents, that can be used in accordance with the teachings of the invention as virus-deactivating substance can be e.g. identified following the teachings of the examples. First, the virus inactivating effect of the candidate substance can be determined as well as the concentration necessary to achieve the desired degree of virus inactivation. Second, it can be confirmed that the candidate substance does not interfere with the amplification performed in (C) when the (optionally pretreated) virus-inactivated sample is subjected to the amplification reaction, optionally in combination with the use of a counteracting substance as disclosed herein.

According to one embodiment, the disinfectant used as virus-deactivating substance is an oxidizing agent selected from the group consisting of
(aa) iodine-releasing agents;
(bb) peroxide-based disinfectants, such as hydrogen peroxide and peroxyacetic acid;
(cc) chlorine-releasing disinfectants, such as sodium hypochlorite.

As is demonstrated in the examples, iodine-releasing agents are particularly useful virus-deactivating substances that can be used to provide the virus-inactivated biological sample in (A). According to one embodiment, the virus-deactivating substance is an iodophore. Iodophores are iodine-releasing agents formed from a complex of iodine with a solubilizing agent in aqueous solutions. Such complexes are advantageous since iodine alone is not stable in water. The released elemental iodine inter alia attacks proteins at the sulfuryl and disulfide bonds. As is demonstrated in the examples, povidone-iodine is a particularly suitable iodophore that can be used as virus-deactivating substance for the purpose of the present invention. It is able to deactivate DNA and RNA viruses such as Coronaviruses very quickly and effectively. Hence, according to an advantageous embodiment, povidone iodine is used as virus-deactivating substance. It was surprising that oxidizing agents such as povidone iodine, in spite of their oxidizing properties, can be used for virus inactivation even though the optionally pretreated virus-inactivated biological sample comprising povidone iodine is directly used in the amplification/reverse transcription amplification, without prior purification of the target nucleic acids. The oxidizing agent is used in the composition in (A) in a concentration that is high enough to assist or achieve virus-inactivation but preferably low enough so that it does not interfere with the amplification reaction/reverse transcription amplification reaction when the desired amount of the optionally pretreated virus-inactivated biological sample is subjected to the amplification reaction. Furthermore, also higher concentrations can be used if a counteracting substance (e.g. a reducing agent) is added in an appropriate amount after virus-inactivation with the oxidizing agent.

Other oxidizing agents with virucidal activity include peroxide-based disinfectants. Examples include but are not limited to hydrogen peroxide and peroxyacetic acid. Such oxidizing agents also target the oxidation of thiol groups and disulfide bonds of proteins and denature them. Hydrogen peroxide is virucidal already at concentrations of 1-3% and can quickly deactivate SARS-CoV. Peroxyacetic acid is more active than hydrogen peroxide against a broad spectrum of pathogens and at lower concentrations. Such peroxy compounds produce hydroxyl radicals that attack different parts of the virus including lipid membrane and proteins. Chlorine-releasing disinfectants such as sodium hypochlorite can also be used for virus-inactivation. At approx. neutral or low pH (e.g. 4-7), the hypochlorite anion gets protonated and exists in equilibrium with hypochlorous acid, which will be the predominant species. It is believed that the acid is the active biocidal agent due to its permeability of membranes and strong oxidizing ability.

As disclosed herein, the skilled person can determine suitable concentrations of the virus-deactivating substance in the composition comprising the biological sample and the virus-deactivating substance that allow to achieve virus-inactivation while allowing the direct amplification in step (C). It is preferred to avoid an unnecessary dilution of the biological sample by the virus-inactivating substance, which in embodiments is a disinfectant such as an oxidizing agent. In non-limiting embodiments, the composition comprising the biological sample and a disinfectant as virus-deactivating substance, comprises the disinfectant, such as an oxidizing agent, in a concentration that lies in the range of 0.001 to 5%, such as 0.01% to 3% or 0.015% to 1%. The percentage can be (w/w) or (v/v) for disinfectants (such as oxidizing agents) added as a liquid or added as a solid. In certain embodiments, the composition comprises the disinfectant, such as an oxidizing agent, in a concentration in the range of or 0.02% to 0.5% or 0.02% to 0.2%. Such concentration ranges are particularly suitable for iodine-releasing agents such as povidone iodine or other iodophores (w/w). The concentration of the disinfectant, such as an oxidizing agent, in the amplification reaction provided in (C) may lie in the range of 0.0005% to 1%, such as 0.001% to 0.5%, 0.0035% to 0.35%, 0.007% to 0.07% or 0.01% to 0.05%. Such concentration ranges are particularly suitable for iodine-releasing agents such as povidone iodine or other iodophores. As disclosed herein the disinfectant is an oxidizing agent, preferably a iodophore such as povidone iodine. Following the teachings of the present application, the skilled person can chose suitable concentrations to effectively inactivate viruses and other pathogens potentially comprised in the biological sample.

As disclosed herein, an inhibitory effect of the disinfectant on the amplification reaction and/or the reverse transcription can be counteracted prior to or during performing the amplification reaction in (C) by addition of at least one substance that can counteract the inhibitory effect of the disinfectant. This is particularly feasible if the virus-deactivating disinfectant is an oxidizing agent and the counteracting substance is a reducing agent. This embodiment is also illustrated in the example wherein an iodophore (here: povidone iodine) is used as disinfectant and a reducing agent (here: TCEP) as reducing agent.

### Surfactants

According to one embodiment, at least one virus-deactivating substance is used which is a surfactant selected from the group consisting of (i) cationic surfactants, (ii) non-ionic surfactants, (iii) anionic surfactants, and (iv) zwitterionic surfactants. As is demonstrated herein, various members of these different classes can be advantageously used as virus-deactivating substance in the method of the present invention because they have strong virucidal activity.

### Cationic Surfactant as Virus-Deactivating Substance

The virus-deactivating substance used to provide the virus-inactivated biological sample may be a cationic surfactant. According to an advantageous embodiment, the cationic surfactant used as virus-deactivating substance is a quaternary ammonium compound, preferably a quaternary ammonium salt. Again, also a mixture of quaternary ammonium compounds may be used for virus inactivation. As is demonstrated in the examples, quaternary ammonium compounds, such as didecyldimethylammonium chloride, can effectively inactivate viruses, such as coronaviruses, especially in respiratory specimens. Furthermore, as also shown in the examples, when used according to the present invention there is no interference with the downstream direct amplification reaction which in preferred embodiments is a reverse transcription amplification reaction.

Quaternary ammonium compounds (QACs) are effective disinfectants and therefore provide suitable virus-deactivating substances for the purpose of the present invention. These compounds are organic-based salts in which a cation is an amino group with four organic substituents on the nitrogen atom and the anion is preferably either a halide or a sulfate. Preferably, the anion is a halide selected from chloride and bromide. The variation of the substituents on the amino group between a combination of alkyl, aryl, and/or heterocycles provides these compounds with a wide range of activity and adaptability. According to one embodiment, the cationic surfactant is an alkyl- or aryl- or alkyl/aryl-quaternary ammonium salt. Also mixtures of quaternary ammonium salts can be used for virus inactivation.

Preferably, the quaternary ammonium compound comprises at least one alkyl group, wherein the chain length is selected from C₁ to C₂₀. According to an advantageous embodiment, at least one of the substituents is a long alkyl chain, while the other three are smaller in size. Such a structure may facilitate the formation of micelles which leads to their biocidal activity through the disintegration (lysing) of the pathogens' membranes and, hence, the loss of their structural integrity. Suitable and preferred embodiments are described in the following.

According to one embodiment, the cationic surfactant used as virus-deactivating substance is a tetraalkylammonium salt. Preferably, one or two alkyl substituent have a chain length selected from C₈ to C₂₀ such as C₁₀ to C₁₈ and two or three alkyl substituents have a chain length selected from C₁ to C₆ such as C₁ to C₄, preferably C₁ or C₂. Suitable embodiments are described in the following.

According to one embodiment, the tetraalkylammonium salt is a dialkyl dimethyl ammonium salt, wherein the chain length of the two alkyl groups is selected from C₈ to C₁₆ such as C₁₀ to C₁₂. The chain length of the two alkyl groups may be the same or different. Dialkyl dimethyl ammonium salts having two alkyl substituents of the same structure are particularly useful as virus-deactivating substance in view of their strong virucidal activity. According to an advantageous embodiment, the virus-deactivating cationic surfactant is didecyldimethylammonium chloride (DDAC), which was also used in the examples. Other cationic surfactants of this class include dimethyldioctadecyl ammonium chloride and dioctadecyldimethylammonium bromide.

According to one embodiment, the tetraalkylammonium salt is an alkyltrimethylammonium salt, wherein preferably, the chain length of the alkyl group is selected from C₈ to C₂₀ such as C₁₀ to C₁₈. The alkyltrimethylammonium salt may be selected from the group consisting of cetrimonium chloride (CTAC) of the formula C₁₉H₄₂ClN, cetrimonium stearate of the formula C₃₇H₇₇NO₂, cetrimonium bromide (CTAB) of the formula C₁₉H₄₂BrN, tetradonium bromide (TTAB or MITMAB) of the formula C₁₇H₃₈BrN, laurtrimonium bromide (DTAB or LTAB) of the formula C₁₅H₃₄BrN or a mixture thereof. Further alkyltrimethylammonium salts include (DTRB), n-dodecyl trimethyl ammonium bromide (DTAB), trimethyl-tetradecyl ammonium bromide, hexadecyl trimethyl ammonium bromide, hexadecyltrimethylammonium bromide (HTAB). Also suitable are the corresponding compounds which comprise a chloride instead of bromide.

According to one embodiment, the cationic surfactant used as virus-deactivating substance is an alkyl/aryl-quaternary ammonium salt, such as an alkylbenzyldimethylammonium salt. The chain length of the alkyl group may be selected from C₈ to C₂₀ or C₈ to C₁₈. The anion is preferably a halide selected from chloride and bromide. Such cationic surfactants are very effective against viruses, including coronaviruses. The alkylbenzyldimethylammonium may be benzalkonium chloride (BAC). The benzalkonium chloride may be of the formula C₆H₅CH₂N(CH₃)₂RCl with R═CₘHₙ, wherein m is selected from 8, 10, 12, 14, 16, and 18 and n is selected from 17, 21, 25, 29, 33, and 37 accordingly. Benzalkonium chlorides are frequently used as active ingredients in disinfectants. According to an advantageous embodiment, the benzalkonium chloride is a mixture of benzalkonium chlorides with different lengths for the alkyl chain, ranging from C₈ to C₁₈, preferably C₁₂ and C₁₄.

An example of an aryl quaternary ammonium salt is cetylpyridinium chloride.

As disclosed herein, the skilled person can determine suitable concentrations of the virus-deactivating cationic surfactant in the composition comprising the biological sample and the virus-deactivating cationic surfactant that allow to achieve virus-inactivation while allowing the direct amplification in step (C). According to one embodiment, the composition comprising the biological sample and the virus-deactivating substance comprises a cationic surfactant as virus-deactivating substance in a concentration that lies in the range of 0.0001% to 3%. The percentage disclosed herein can be (w/w) or (v/v) for cationic surfactants added as a liquid or added as a solid and preferably refers to (w/w). In embodiments, the composition comprises the cationic surfactant in a concentration that lies in the range of 0.0005% to 1%, 0.001% to 0.5%, 0.005% to 0.1% and 0.01% to 0.05%. Such concentration ranges are particularly suitable for quaternary ammonium salts, such as DDAC, which are highly effective for virus inactivation already at low concentrations whereby a dilution of the biological sample is avoided. The concentration of the cationic surfactant in the amplification reaction provided in (C) may lie in the range of 0.0001% to 0.1%, such as 0.0002% to 0.05%, 0.00025% to 0.025%, optionally 0.0025% to 0.01%. Following the teachings of the present application, the skilled person can chose suitable cationic surfactant concentrations to effectively inactivate viruses potentially comprised in the biological sample. As is demonstrated in the examples, small amounts of cationic surfactant are often sufficient to achieve virus inactivation which is advantageous to avoid a dilution of the sample.

According to one embodiment, an inhibitory effect of the cationic surfactant on the amplification reaction and/or the reverse transcription is counteracted prior to or during performing the amplification reaction in (C) by addition of at least one substance that can counteract the inhibitory effect of the cationic surfactant. According to one embodiment, the at least one substance that can counteract the inhibitory effect of the cationic surfactant is a surfactant. According to one embodiment, the at least one counteracting surfactant is added after virus-inactivation in (A). It may be added in pretreatment step (B) and/or may be included in the amplification reaction of (C). According to one embodiment, the counteracting surfactant is not a cationic surfactant. The counteracting surfactant may be selected from anionic surfactants, non-ionic surfactants and zwitterionic surfactant. In an advantageous embodiment, the surfactant that counteracts the inhibitory effect of the cationic surfactant is an anionic surfactant, such as SDS. This embodiment is particularly feasible wherein the cationic surfactant used as virus-deactivating substance is a quaternary ammonium salt. As demonstrated in the examples, the inhibitory effect of such cationic surfactant (here: didecyldimethylammonium chloride), can be effectively counteracted by an anionic surfactant (here: SDS). The counteracting substance, such as the counteracting surfactant, is used in a concentration where it can counteract the inhibitory effect of the cationic surfactant while not substantially inhibiting the amplification reaction/the reverse transcription amplification reaction. Suitable concentrations can be determined following the principles of the examples by testing different concentration series.

### Non-Ionic Surfactant as Virus-Deactivating Substance

The virus-deactivating substance used to provide the virus-inactivated biological sample may furthermore be a non-ionic surfactant. According to an advantageous embodiment, the non-ionic surfactant used as virus-deactivating substance is an alcohol ethoxylate or an alkyl glycoside. Also a mixture of different alcohol ethoxylates, a mixture of different alkyl glycosides or a mixture of one or more alcohol ethoxylates and one or more alkyl glycosides may be used for virus inactivation.

Preferably, a non-ionic surfactant having strong virucidal activity is used in a concentration where it does not inhibit the amplification/reverse transcription amplification in (C). In advantageous embodiments, a non-ionic surfactant is used as virus-deactivating substance in a concentration that meets the DIN EN 14476: 2019-10 standard.

### Alcohol Ethoxylate

According to one embodiment, the virus-deactivating substance is an alcohol ethoxylate selected from the group consisting of 2-ethyl hexanol ethoxylated propoxylated copolymers, seed oil alcohol alkoxylates, linear or branched alkyl polyethylene glycol ethers, such as C₁₀-Guerbet-based alkyl polyethylene glycol ethers, polysorbates and polyoxyethylene alkyl phenyl ethers.

In one embodiment, the virus-deactivating substance is an alcohol ethoxylate selected from the group consisting of 2-ethyl hexanol ethoxylated propoxylated copolymers, seed oil alcohol alkoxylates and C₁₀-Guerbet-based alkyl polyethylene glycol ethers. In embodiments, these alcohol ethoxylates comprise three to twelve ethoxylated moieties, such as three ((EO)₃), four ((EO)₄), six ((EO)₆), seven ((EO)₇), or nine ((EO)₉) ethoxylated moieties (sometimes also referred to as moles). More preferred alcohol ethoxylates from the group consisting of 2-ethyl hexanol ethoxylated propoxylated copolymers, seed oil alcohol alkoxylates and C₁₀-Guerbet-based alkyl polyethylene glycol ethers comprise four ((EO)₄), seven ((EO)₇) or nine ((EO)₉) ethoxylated moieties. Most preferred alcohol ethoxylates from the group consisting of 2-ethyl hexanol ethoxylated propoxylated copolymers, seed oil alcohol alkoxylates and C₁₀-Guerbet-based alkyl polyethylene glycol ethers comprise seven ((EO)₇) or nine ((EO)₉) ethoxylated moieties.

According to one embodiment, the alcohol ethoxylate is a polysorbate. Exemplary polysorbates are polyoxyethylene (20) sorbitan monolaurate (Tween® 20), polyoxyethylene (20) sorbitan monopalmitate (Tween® 40), polyoxyethylene (20) sorbitan monostearate (Tween® 60), polyoxyethylene (20) sorbitan tristearate (Tween® 65) and polyoxyethylene (20) sorbitan monooleate (Tween® 80). Also mixture of different polysorbates may be used.

Further polysorbates are also described elsewhere herein. Polysorbates are also described elsewhere herein and may also be used as counteracting substance. Where a polysorbate of weaker virucidal activity such as polyoxyethylene (20) sorbitan monolaurate (Tween® 20) is used as virus-deactivating substance, it is preferably used in combination with a further virus-deactivating substance of stronger virucidal activity and/or in combination with heating in order to provide a virus-inactivated biological sample in (A). In other embodiments, the virus-deactivating substance is not a polysorbate.

Also suitable is an alcohol ethoxylate which is a polyoxyethylene alkyl phenyl ether. According to one embodiment, the non-ionic surfactant is a polyethylene glycol p-(1,1,3,3-tetramethylbutyl)-phenyl ether, e.g. having the chemical formula of C₁₄H₂₂O(C₂H₄O)ₙ, wherein n is 9-10 (Triton X-100, CAS: 9002-93-1). Polyoxyethylene alkyl phenyl ether are also described elsewhere herein.

According to one embodiment, the alcohol ethoxylate is selected from the group consisting of seed oil alcohol alkoxylates and 2-ethyl hexanol ethoxylated propoxylated copolymers. In embodiments, the alcohol ethoxylate comprises three to twelve ethoxylated moieties, such as three ((EO)₃), four ((EO)₄, six ((EO)₆), seven ((EO)₇), or nine ((EO)₉) ethoxylated moieties. As is demonstrated in the examples, such non-ionic surfactants are advantageous as virus-deactivating substances. They may also be used in combination with a further virus-deactivating substance of even stronger virucidal activity.

According to a preferred embodiment, a seed oil alcohol alkoxylate is used as virus-deactivating substance. The seed oil alcohol alkoxylate may be of the formula X(C₃H₆O)ₘ(C₂H₄O)ₙ, wherein X is an aromatic or aliphatic C₆ to C₁₂ alkyl group, which is linear, branched or cyclic and optionally further substituted, m is 3 or 4 and n is an integer from 4 to 9. Preferably n is 4, 7 or 9. The skilled person is aware of the fact that surfactants such as seed oil alcohol alkoxylates are mixtures due to their origin. Accordingly, X may vary between C₆ and C₁₂ alkyl groups, m can vary from 3 to 4 and n is in embodiments a set value of 4, 7 or 9. According to one embodiment, the seed oil alcohol alkoxylate has a HLB value in the range of 5 to 15, e.g. 7 to 13. It may have a CMC (critical micelle concentration) at 25° C. less or equal to (s) 50 ppm. Preferred seed oil alcohol alkoxylates are those which are commercially available from DOW*, e.g. ECOSURF SA-9 (CAS: 68937-66-6), ECOSUR SA-7 or ECOSURF SA-4. Preferred seed oil alcohol alkoxylates are ECOSURF SA-7 and ECOSURF SA-9. Furthermore, mixtures comprising such seed oil alcohol alkoxylates can be used for virus inactivation.

According to an advantageous embodiment, the seed oil alcohol alkoxylate used as virus-deactivating non-ionic surfactant is of the formula X(C3H6O)m(C2H4O)n, wherein X is an aromatic or aliphatic C6 to C12 alkyl group, which is linear, branched or cyclic and optionally further substituted, m is 3 or 4 and n is 9. In embodiments, said seed oil alcohol alkoxylate has a HLB value in the range of 11 to 13 and a CMC (at 25° C.) in the range of 20 to 25 ppm. ECOSURF SA-9 is an example of such seed oil alcohol alkoxylate of the general formula X(C₃H₆O)ₘ(C₂H₄O)ₙ, wherein X is an aromatic or aliphatic C₆ to C₁₂ alkyl group, which is linear, branched or cyclic and optionally further substituted, m is 3 or 4 and n is 9. It usually has a HLB value of 11.1 and a CMC (at 25° C.) at 22 ppm. As demonstrated in the examples, a seed oil alcohol alkoxylate of the formula X(C3H6O)m(C2H4O)n, wherein X is an aromatic or aliphatic C₆ to C₁₂ alkyl group, which is linear, branched or cyclic and optionally further substituted, m is 3 or 4 and n is 9, is particularly suitable as virus-deactivating non-ionic surfactant as it can inactivate viruses, such as coronaviruses. Furthermore, the examples also show that such seed oil alcohol alkoxylates when used according to the invention do not interfere with a conventional amplification reaction and/or reverse transcription amplification reaction, such as (RT-)PCR, even when used at very high concentrations.

According to one embodiment, the seed oil alcohol alkoxylate used as virus-deactivating non-ionic surfactant is of the formula X(C₃H₆O)ₘ(C₂H₄O)ₙ, wherein X is an aromatic or aliphatic C₆ to C₁₂ alkyl group, which is linear, branched or cyclic and optionally further substituted, m is 3 or 4 and n is 7. In embodiments, said seed oil alcohol alkoxylate has a HLB value in the range of 9 to 11, preferably 9 to 10 and a CMC (at 25° C.) in the range of 15 to 20 ppm. ECOSURF SA-7 is an example of a seed oil alcohol alkoxylate of the general formula X(C₃H₆O)ₘ(C₂H₄O)ₙ, wherein X is an aromatic or aliphatic C₆ to C₁₂ alkyl group, which is linear, branched or cyclic and optionally further substituted, m is 3 or 4 and n is 7. It usually has a HLB value of 9.7 and a CMC (at 25° C.) at 17 ppm.

According to one embodiment, the seed oil alcohol alkoxylate used as virus-deactivating non-ionic surfactant is of the formula X(C₃H₆O)ₘ(C₂H₄O)ₙ, wherein X is an aromatic or aliphatic C₆ to C₁₂ alkyl group, which is linear, branched or cyclic and optionally further substituted, m is 3 or 4 and n is 4. In embodiments, said seed oil alcohol alkoxylate has a HLB value in the range of 7 to 8, such as 7.5, and a CMC (at 25° C.) lower than 5 ppm, optionally 0 ppm. ECOSURF SA-4 is a seed oil alcohol alkoxylate of the general formula X(C₃H₆O)ₘ(C₂H₄O)ₙ, wherein X is an aromatic or aliphatic C₆ to C₁₂ alkyl group, which is linear, branched or cyclic and optionally further substituted, m is 3 or 4 and n is 4. It usually has a HLB value of 7.5 and a CMC (at 25° C.) at 0 ppm.

According to further embodiments, the non-ionic surfactant used as virus-deactivating substance is a 2-ethyl hexanol ethoxylated propoxylated copolymer. According to one embodiment, the 2-ethyl hexanol ethoxylated propoxylated copolymer has a HLB value in the range of 5 to 15. It may have a CMC (at 25° C.) in the range of 350 to 1200 ppm. It can be derived from the general formula C₈H₁₈O(C₃H₆O)ₘ(C₂H₄O)ₙ, wherein m is an integer from 1 to 12 and n is an integer from 3 to 9. Preferably m is an integer from 1 to 8 and n is 3, 6 or 9. The skilled person is aware of the fact that non-ionic surfactants such as 2-ethyl hexanol ethoxylated propoxylated copolymers can be mixtures due to their polymeric nature, wherein m can vary from 1 to 12 and wherein n is a set value of 3, 6 or 9. Such 2-ethyl hexanol ethoxylated propoxylated copolymers can have a HBL value between 5 and 15, such as between 7 and 13. Furthermore, they can have a CMC (critical micelle concentration) at 25° C. in the range of 350 to 1200 ppm. Preferred 2-ethyl hexanol ethoxylated propoxylated copolymers are those which are commercially available from DOW*e, e.g. ECOSURF EH-9 (CAS: 64366-70-7), ECOSURF EH-6 or ECOSURF EH-3.

According to a further embodiment, the 2-ethyl hexanol ethoxylated propoxylated copolymer used as virus-deactivating non-ionic surfactant is of the formula C₈H₁₈O(C₃H₆O)ₘ(C₂H₄O)ₙ, wherein m is an integer from 1 to 12 and n is an integer from 3 to 9.

According to one embodiment, the 2-ethyl hexanol ethoxylated propoxylated copolymer is of the formula C₈H₁₈O(C₃H₆O)ₘ(C₂H₄O)ₙ, wherein m is an integer from 1 to 12, preferably an integer from 1 to 8 and n is 9. In embodiments, said 2-ethyl hexanol ethoxylated propoxylated copolymer has a HLB value in the range of 12 to 13, such as 12.5, and a CMC (at 25° C.) in the range of 1000 to 1100 ppm. ECOSURF EH-9 is a 2-ethyl hexanol ethoxylated propoxylated copolymer of the general formula C₈H₁₈O(C₃H₆O)ₘ(C₂H₄O)ₙ, wherein m is an integer from 1 to 12, preferably m is an integer from 1 to 8 and n is 9. It has a HLB value of 12.5 and a CMC (at 25° C.) at 1066 ppm. As demonstrated in the examples, a 2-ethyl hexanol ethoxylated propoxylated copolymer of the formula C₈H₁₈O(C₃H₆O)ₘ(C₂H₄O)ₙ, wherein m is an integer from 1 to 12, preferably an integer from 1 to 8 and n is 9, when used according to the invention does not interfere with reverse transcription and/or amplification reaction, such as (RT-)PCR.

According to one embodiment, the 2-ethyl hexanol ethoxylated propoxylated copolymer is of the formula C₈H₁₈O(C₃H₆O)ₘ(C₂H₄O)ₙ, wherein m is an integer from 1 to 12, preferably an integer from 1 to 8 and n is 6. In embodiments, said 2-ethyl hexanol ethoxylated propoxylated copolymer has a HLB value in the range of 10 to 11, such as 10.8, and a CMC (at 25° C.) in the range of 900 to 950 ppm. ECOSURF EH-6 is a 2-ethyl hexanol ethoxylated propoxylated copolymer of the general formula C₈H₁₈O(C₃H₆O)ₘ(C₂H₄O)ₙ, wherein m is an integer from 1 to 12, preferably m is an integer from 1 to 8 and n is 6. It has a HLB value of 10.8 and a CMC (at 25° C.) at 914 ppm.

According to one embodiment, the 2-ethyl hexanol ethoxylated propoxylated copolymer is of the formula C₈H₁₈O(C₃H₆O)ₘ(C₂H₄O)ₙ, wherein m is an integer from 1 to 12, preferably an integer from 1 to 8, and n is 3. In embodiments, said 2-ethyl hexanol ethoxylated propoxylated copolymer has a HLB value in the range of 7 to 8.5, such as about 8, and a CMC (at 25° C.) in the range of 450 to 500 ppm. ECOSURF EH-3 is a 2-ethyl hexanol ethoxylated propoxylated copolymer of the general formula C₈H₁₈O(C₃H₆O)ₘ(C₂H₄O)ₙ, wherein m is an integer from 1 to 12, preferably m is an integer from 1 to 8 and n is 3. It usually has a HLB value of 7.9 and a CMC (at 25° C.) at 480 ppm.

### Polysorbates and Polyoxyethylene Alkyl Phenyl Ethers

As also noted elsewhere herein, the virus-deactivating substance may be selected from the group consisting of polyoxyethylene fatty acid esters, in particular polyoxyethylene sorbitan fatty acid esters and polyoxyethylene alkylphenyl ether, optionally wherein the polyoxyethylene alkyl phenyl ether is selected from the group consisting of polyoxyethylene nonylphenyl ether and polyoxyethylene isooctylphenyl ether.

According to one embodiment, the non-ionic surfactant is a polyoxyethylene fatty acid ester, comprising
a fatty acid derived from laureate, palmitate, stearate and oleate,
a polyoxyethylene component containing from 2 to 150, 4 to 100, 6 to 50 or 6 to 30 (CH₂CH₂O) units.

Polyoxyethylene sorbitan fatty acid esters, also referred to as polysorbates, are particularly preferred as non-ionic surfactant as virus-deactivating substance. The non-ionic surfactant may be selected from polyoxyethylene (20) sorbitan monolaurate (e.g. Tween20), polyoxyethylene (4) sorbitan monolaurate (e.g. Tween21), polyoxyethylene (40) sorbitan monopalmitate (e.g. Tween40), polyoxyethylene (60) sorbitan monostearate (e.g. Tween60), polyoxyethylene (4) sorbitan monostearate (e.g. Tween61), polyoxyethylene (20) sorbitan tristearate (e.g. Tween65), polyoxyethylene (40) sorbitan monooleate (e.g. Tween80), polyoxyethylene (5) sorbitan monooleate (e.g. Tween81), polyoxyethylene sorbitan trioleate (e.g. Tween85), and polyoxyethylen (20) sorbitan monoisostearate. According to a preferred embodiment, the polyoxyethylene fatty acid ester is selected from polysorbate 20, polysorbate 40, polysorbate 60 and polysorbate 80. In embodiments, said non-ionic surfactant is used in combination with at least one additional virus-deactivating substance of stronger virucidal activity and/or heating in order to provide a virus-inactivated biological sample in (A). These non-ionic surfactants may also be used as counteracting substance, e.g. for inhibitory ionic surfactants used as virus-deactivating substance. As noted, in certain embodiments, the virus-deactivating substance is not a polysorbate.

In one embodiment, the non-ionic surfactant is a polyoxyethylene alkyl phenyl ether. The polyoxyethylene alkylphenyl ether may have an alkyl group containing from five to 15 carbon atoms, such as 6 to 10 carbon atoms. Also encompassed are branched or unbranched C₇- to C₁₀-alkyl groups, such as branched or unbranched C₈- and C₉-alkyl groups, e.g. isooctyl groups and nonyl groups. The polyoxyethylene alkylphenyl ether may be selected from the group consisting of polyoxyethylene nonylphenyl ether and polyoxyethylene isooctylphenyl ether. It may be Triton X 100.

### Linear and Branched Alkyl Polyethylene Glycol Ethers

According to one embodiment, the virus-deactivating substance is a linear or branched alkyl polyethylene glycol ether, preferably of the general formula C₁₃H₂₁O(CH₂CH₂O)_{y}H, wherein y is an integer in the range of 5 to 14. These non-ionic surfactants are also suitable as virus-deactivating substance.

According to one embodiment, the virus-deactivating substance is a C₁₀-Guerbet-based alkyl polyethylene glycol ether, preferably selected from the general formula C₁₀H₂₁(CH₂CH₂O)ₓH, wherein x is an integer in the range of 3 to 10 or 14. According to one embodiment, the virus-deactivating substance is a C₁₀-Guerbet-based alkyl polyethylene glycol ether is of the formula C₁₀H₂₁(CH₂CH₂O)₉H and has a HBL value in the range of 14 to 15, preferably of 14.5. Further non-ionic surfactants suitable for use as virus-deactivating substance are alcohol ethoxylates which are linear or branched alkyl polyethylene glycol ethers, such as Coo-Guerbet-based alkyl polyethylene glycol ethers. Coo-Guerbet-based alkyl polyethylene glycol ethers can be derived from the general formula C₁₀H₂₁(CH₂CH₂O)ₓH, wherein x is an integer in the range of 3 to 10 or 14. Preferably x is 9. C₁₀-Guerbet-based alkyl polyethylene glycol ethers can have a HBL value between 8 and 17, such as 8.5 to 16.5. Preferred C₁₀-Guerbet-based alkyl polyethylene glycol ethers are those which are commercially available from BASF®, e.g. Lutensol XP types. Lutensol XP 90 is a C₁₀-Guerbet-based alkyl polyethylene glycol ether of the general formula C₁₀H₂₁(CH₂CH₂O)ₓH, wherein x is 9. It usually has a HLB value of approximately 14.5.

Alternative linear or branched alkyl polyethylene glycol ethers can be derived from the general formula C₁₃H₂₁O(CH₂CH₂O)_{y}H, wherein y is an integer in the range of 5 to 14.

### Alkyl Glycoside

Further non-ionic surfactants suitable for use as virus-deactivating substance are alkyl glycosides. The virus-deactivating substance may be selected from the group consisting of decyl polyglucoside, decyl β-D glucopyranoside, lauryl glucoside, n-octyl β-D glucopyranoside. Also mixtures thereof may be used. Aqueous preparations thereof are commercially available and suitable as non-ionic surfactants acting as a virus-deactivating substance. Embodiments of alkyl glycoside-based non-ionic surfactants are commercially available. These include Triton™ CG-110 (CAS: 68515-73-1) from DOW, which is an aqueous preparation of D-glucopyranose oligomeric decyl octyl glycosides (˜58-62 wt-%); Mackol™ DG from Rhodia Inc., which is an aqueous preparation of D-glucopyranose oligomeric C₁₀₋₁₆ alkyl glycosides (CAS: 110615-47-9) (˜53-57 wt-%); and APN® 325 N (BASF), which is an aqueous preparation of D-glucopyranose oligomeric C₉₋₁₁-alkyl glycosides (CAS: 132778-08-6).

Other surfactants that can be used as virus-deactivating substances are mixtures of fatty alcohols, free fatty acids and fatty esters. Commercial products include SAFE CARE® SC-1000 (HLB: 13.85) from GEMTECK® products.

As disclosed herein, the skilled person can determine suitable concentrations of the virus-deactivating non-ionic surfactant in the composition comprising the biological sample and the virus-deactivating non-ionic surfactant that allow to achieve virus-inactivation while allowing the direct amplification in step (C). According to one embodiment, the composition comprising the biological sample and the virus-deactivating substance comprises a non-ionic surfactant as virus-deactivating substance in a concentration that lies in the range of 0.1% to 25%, such as 0.1% to 15% or 0.2% to 10%. To avoid dilution of the biological sample, the concentration of the virus-deactivating non-ionic surfactant is preferably in the range of 0.1% to 5%, such as 0.2% to 2% or 0.3% to 1.5%. As demonstrated in the examples such lower concentrations are in particular suitable for seed oil alcohol ethoxylates. The percentage disclosed herein can be (v/v) or (w/w) for non-ionic surfactants added as a liquid or added as a solid. As non-ionic surfactants can be conveniently added as a liquid, the percentages disclosed herein in particular refer to (v/v). According to one embodiment, the virus-deactivating substance is a non-ionic surfactant and wherein the concentration of the non-ionic surfactant in the amplification reaction provided in (C) lies in the range of 0.01% to 10%, 0.02% to 5%, and 0.03% to 3%. As disclosed in the examples, the amplification/reverse transcription amplification can be performed also in the presence of a high concentration of a non-ionic surfactant. However, the concentration is preferably low to avoid dilution effects due to the presence of the non-ionic surfactant. In embodiments, the virus-deactivating non-ionic surfactant is comprised in the amplification reaction provided in (C) in a concentration that lies in the range of 0.05% to 1%, such as 0.05% to 0.7%, optionally 0.1% to 0.5%. Following the teachings of the present application, the skilled person can chose suitable non-ionic surfactant concentrations to effectively inactivate viruses potentially comprised in the biological sample.

### Anionic Surfactant as Virus-Deactivating Substance

According to one embodiment, the virus-deactivating substance is an anionic surfactant.

Anionic surfactants, include, but are not limited to sodium dodecyl sulfate (SDS), lithium dodecyl sulfate, sodium octyl sulfate, sodium dodecyl sulfonate, sodium decyl sulfonate, sodium octyl sulfonate, dodecylbenzene sulfonic acid (DDBSA), N-lauroyl sarcosine, sodium cholate, sodium deoxycholate and caprylic acid. Optionally, the anionic surfactant is selected from sodium dodecyl sulfate, N-lauroyl sarcosine and caprylic acid. Two or more anionic surfactants may also be used for virus inactivation. The use of SDS is particularly suitable, as it has a strong virus-inactivating effect.

As disclosed herein, the skilled person can determine suitable concentrations of the virus-deactivating anionic surfactant in the composition comprising the biological sample and the virus-deactivating anionic surfactant that allow to achieve virus-inactivation while allowing the direct amplification in step (C). It is also referred to the concentration ranges indicated for the cationic surfactant which provide guidance for suitable concentration ranges for the anionic surfactant. Furthermore, inhibitory effects of the anionic surfactant on the polymerase/reverse transcriptase may be counteracted. According to one embodiment, an inhibitory effect of the anionic surfactant on the amplification reaction and/or the reverse transcription is counteracted prior to or during performing the amplification reaction in (C).

This can be achieved by addition of at least one substance that can counteract the inhibitory effect of the anionic surfactant. According to one embodiment, the at least one substance that can counteract the inhibitory effect of the anionic surfactant is also a surfactant. The counteracting surfactant can be added after providing the virus-inactivated biological sample in (A). The counteracting surfactant may be added in pretreatment step (B) and/or may be included in the amplification reaction of (C). The surfactant serving the purpose to counteract the inhibitory effect of an anionic surfactant is preferably not an anionic surfactant. According to an advantageous embodiment, the surfactant that counteracts the inhibitory effect of the anionic surfactant is a non-ionic surfactant or a cationic surfactant. According to a preferred embodiment, the anionic surfactant used as virus-deactivating substance is SDS and the surfactant that counteracts the inhibitory effect of SDS is an quaternary ammonium salt. Suitable quaternary ammonium salts are described herein, such as didecyldimethylammonium chloride. As demonstrated in the examples, the inhibitory effect of an anionic surfactant, such as SDS, can be counteracted by either a cationic surfactant, such as didecyldimethylammonium chloride, or by a non-ionic surfactant, such as a polysorbate. The use of a non-ionic surfactant as counteracting substance is advantageous as numerous non-ionic surfactants do not inhibit the polymerase/reverse transcriptase even at rather high concentrations.

### Zwitterionic Surfactant as Virus-Deactivating Substance

According to one embodiment, the virus-deactivating substance is a zwitterionic surfactant. An amine oxide-based zwitterionic surfactant may be used as virus-deactivating substance. According to an advantageous embodiment, the amine oxide-based zwitterionic surfactant comprises a C8 to C18 alkyl group, such as a C10 to C16 alkyl group or a C12 to C14 alkyl group. In embodiments, it comprises a C12 alkyl group.

According to one embodiment, the amine oxide-based zwitterionic surfactant is an alkyldimethyl amine oxide. Alkyldimethyl amine oxides have been shown to have antimicrobial activity. According to an advantageous embodiment, the alkyldimethyl amine oxide comprises a C8 to C18 alkyl group, optionally a C10 to C16 alkyl group or a C12 to C14 alkyl group, such as a C12 alkyl group. In a preferred embodiment, the amine oxide-based zwitterionic surfactant is lauryldimethylamine N-oxide (LDAO). As demonstrated in the examples, LDAO does not interfere with (RT-)PCR when used in an appropriate concentration. The skilled person can choose appropriate concentrations following the general guidance provided in this application. Other amine oxide-based zwitterionic surfactants include dimethylamineoleyl oxide and dimethyltetradecyl amine oxide.

According to one embodiment, the amine oxide-based zwitterionic surfactant is a cyclic amine oxide. According to one embodiment, the amine oxide-based zwitterionic surfactant is selected from the group consisting of N-lauryl morpholine oxide, N-lauryl piperidine oxide and N-lauryl-3-methyl piperidine oxide. According to one embodiment, the amine oxide-based zwitterionic surfactant is a 2-alkoxy-N,N-dimethyl amine N-oxide. According to one embodiment, the amine oxide-based zwitterionic surfactant is 2-lauryloxy-N,N-dimethylethyl amine N-oxide. The amine oxide-based zwitterionic surfactant is may also be an alkyl benzene-derived amine oxide such as an alkylbenzene sulfonamide amine oxide.

Alkyl betaine-based zwitterionic surfactants may also be used as virus-deactivating substance as alkyl betaines have been shown to have antimicrobial activity. The alkyl betaine-based zwitterionic surfactant may comprise a C8 to C18 alkyl group. According to an advantageous embodiment, the alkyl betaine-based zwitterionic surfactant is oleyl betaine.

According to one embodiment, a mixture of an alkyldimethyl amine oxide and an alkyl betaine, such as oleyldimethylamine oxide and oleyl betaine, is used for virus-deactivation.

Also other zwitterionic surfactants with virucidal activity may be used for the purpose of the present invention.

As disclosed herein, the skilled person can determine suitable concentrations of the virus-deactivating zwitterionic surfactant in the composition comprising the biological sample and the virus-deactivating zwitterionic surfactant that allow to achieve virus-inactivation while allowing the direct amplification in step (C). According to one embodiment, the composition comprising the biological sample comprises a zwitterionic surfactant as virus-deactivating substance in a concentration that lies in the range of 0.001% to 5%, such as 0.01% to 3%, 0.01% to 2.0%, 0.02% to 1.5% (v/v) or 0.05% to 1.5%. The percentage disclosed herein can be (w/w) or (v/v) for zwitterionic surfactants added as a liquid or added as a solid and preferably is (w/w). According to one embodiment, the virus-deactivating substance is a zwitterionic surfactant and wherein the concentration of the zwitterionic surfactant in the amplification reaction provided in (C) lies in the range of 0.002% to 1%, such as 0.007% to 0.7%, 0.01% to 0.5% or 0.01% to 0.1%. The skilled person can chose suitable zwitterionic surfactant concentrations to effectively inactivate viruses potentially comprised in the biological sample. Furthermore, a counteracting substance may be used, if necessary.

### Organophosphorous Compound as Virus-Deactivating Substance

According to a further embodiment, the virus-deactivating substance is an organophosphorous compound, such as tri-n-butyl phosphate (TBP). According to one embodiment, at least one virus-deactivating substance is used that is selected from the group consisting of quaternary ammonium salts, seed oil alcohol alkoxylates, 2-ethyl hexanol ethoxylated propoxylated copolymers, polyoxyethylene alkyl phenyl ether, SDS and an amine oxide-based zwitterionic surfactant comprising a C₁₀ to C₁₆ alkyl group.

The virus-deactivating effect of a substance can be determined using methods available in the art, including the tests described in the examples. The virus-deactivating effect of substances disclosed herein has also been described in the art see (e.g. Welch et al. (2020), Al-Sayah, 2020 and US 2016/0333046). Hence, the substances disclosed herein can be advantageously used for virus-inactivation in the method according to the present invention.

### The Biological Sample

The method according to the first aspect enables the rapid and safe processing of biological samples that comprise or potentially comprise one or more viruses. The technology of the invention provides virus-inactivated biological samples for direct virus testing by amplification, including reverse-transcription amplification, wherein such amplification can be carried out in a rapid and robust manner. A prior purification of the nucleic acids is not necessary. In principle, any biological sample commonly used for virus testing can be processed using the method according to the present invention. Non-limiting and preferred examples for biological samples that can be processed with the present invention are described in the following.

The biological sample may be derived from a human and may thus be a human sample. This is particularly advantageous for diagnostic applications that rely on the amplification based detection of one or more target nucleic acids e.g. in order to identify the infection with one or more pathogens, such as one or more viruses, that can be determined based on the presence or absence of a target nucleic acid.

The biological sample can be a bodily sample and preferably is a cell-containing sample. Common bodily samples used for pathogen detection include, but are not limited to, swab samples, smear samples, blood and blood derived samples such as plasma or serum, urine, saliva, aspirates, lymphatic fluid, liquor, cerebrospinal fluid, synovial fluid, interstitial fluid, ascites, milk, bronchial lavage, amniotic fluid, semen/seminal fluid, body secretions, nasal secretions, vaginal secretions, wound secretions and excretions.

According to advantageous core embodiments, the biological sample is a respiratory specimen. The respiratory specimen may be collected from the upper or lower respiratory tract and is preferably collected from the upper respiratory tract. Such biological samples are particularly suitable for the detection of viruses, including RNA viruses, such as in particular an acute respiratory syndrome-related coronavirus. As is demonstrated in the examples, the method according to the first aspect is particularly suitable to provide virus-inactivated respiratory specimen samples for direct amplification based detection of contained viral targets, such as RNA target nucleic acids, without prior nucleic acid isolation. The biological sample may thus be an oral sample, a nasal sample, a nasopharyngeal sample, an oropharyngeal sample, a throat sample or a combination of the foregoing. In a particular embodiment, the biological sample is selected from saliva, sputum, spittle, mucus, drool, bronchoalveolar lavage, pharynx secretions, nasal secretions, nasopharyngeal secretions, salivary secretions, a swab or smear derived from mouth, nose and/or throat and a combination of the foregoing. According to a particularly preferred embodiment, the biological sample is selected from nasopharyngeal, oropharyngeal and nasal samples, preferably selected from a nasopharyngeal, oropharyngeal or nasal swab, smear or wash/aspirate samples, more preferably selected from swab or smear samples.

In preferred embodiments, the biological sample is a swab sample, preferably contained in a medium, and the at least one target nucleic acid is a viral RNA. The viral RNA may be derived from a virus selected from a severe acute respiratory syndrome-related coronavirus, preferably severe acute respiratory syndrome coronavirus 2 (SARS-CoV-2) or other severe acute respiratory syndrome coronavirus (SARS-CoV or SARS-CoV-1), more preferably SARS-CoV-2. In particular, the biological sample may be a nasopharyngeal, oropharyngeal or nasal swab sample and the target nucleic acid is a viral RNA derived from a coronavirus, preferably a human coronavirus, such as in particular severe acute respiratory syndrome coronavirus 2 (SARS-CoV-2). In a further embodiment, the biological sample is saliva, sputum or mucus and the target nucleic acid is a viral RNA derived from a coronavirus, preferably a human coronavirus, such as in particular severe acute respiratory syndrome coronavirus 2 (SARS-CoV-2). As disclosed herein, also two or more viruses and/or other pathogens can be detected using the technology of the invention.

### The Transport Medium

As disclosed herein, many biological samples are upon collection placed into a medium (e.g. for transport or storage). They may also be transported in dry form and are then transferred to a medium prior to processing the biological sample for amplification based detection of the one or more viral target nucleic acids. It is a particular advantage of the method of the present invention that it allows to directly process a biological sample contained in a medium, such as a transport medium, for amplification based detection of at least one target nucleic acid without prior target nucleic acid purification or removal of the medium. As is demonstrated in the examples, the method according to the invention is robust and advantageously allows to prepare and process virus-inactivated biological samples contained in media for amplification based detection of at least one target nucleic acid without prior target nucleic acid purification. The biological sample contained in medium can thus be directly processed and there is no need to remove the medium in advance. This considerably simplifies and accelerates the workflow for high throughput testing.

In certain embodiments, the biological sample is collected from the subject (e.g. a human) and directly transferred into a medium, such as a transport medium. In embodiments disclosed herein, said medium comprises at least one virus-deactivating substance. E.g. the biological sample may be collected by swabbing and the swab is placed into a transport medium, optionally comprising at least one virus-deactivating substance, prior to transportation and/or storage. In other embodiments, the biological sample is collected from the subject and after a delay, which optionally comprises storing and/or transporting the sample, is the biological sample contacted with the medium, optionally comprising at least one virus-deactivating substance, to provide a biological sample contained in medium, optionally comprising at least one virus-deactivating substance. E.g. the biological sample may be collected into a container without any liquid and transported. Such “dry” collection of a biological sample, such as a swab sample, is sometimes performed in the situation of a pandemic where a large number of samples are collected and there is a shortage of transport media. In this case, the biological sample is preferably contacted with a liquid medium, such as physiological salt solution, optionally comprising at least one virus-deactivating substance, to receive the biological sample in a medium, optionally comprising at least one virus-deactivating substance.

According to one embodiment, the medium containing the biological sample is a transport medium. Preferably, the medium is a transport medium for swab and/or smear samples. Suitable embodiments of such transport media are known to the skilled person and furthermore described herein.

The medium is preferably an aqueous solution. The medium may be a saline solution suitable to keep the osmotic pressure in cells comprised in the biological sample when the medium is in contact with the biological sample. The medium may stabilize cells and/or viral particles comprised in the biological sample. This supports the protection of the target nucleic acids by inhibiting e.g. the release of nucleases from cells contained in the biological sample and preserving viral particles that contain the target nucleic acids. Using such media for receiving the biological sample is advantageous as it preserves the targets during transportation/storage as is well-known known in the art. The medium may also stabilize the at least one target nucleic acid against degradation. It is preferred that the medium for receiving the biological sample does not result in cross-linking or other fixation of the contained nucleic acids that could hamper and thus impair the subsequent direct amplification based detection of the one or more target nucleic acids in the optionally pretreated virus-inactivated biological sample due to the cross-links/fixation.

According to one embodiment, the medium that comprises the biological sample and optionally the at least one virus-deactivating substance is a salt containing solution. The medium is in embodiments a salt containing solution. The total salt concentration in the medium may lie in a range of 50 mM to 250 mM, such as 75 mM to 225 mM or 100 mM to 200 mM. The total salt concentration in the medium may lie in a range of 120 mM to 175 mM or 125 mM to 150 mM. Many common media used for the collection of biological samples, such as swab samples, have a salt concentration in the aforementioned range. Many common transport media used for collecting biological samples such as swab samples comprise Hank's balanced salt solution as core component. In embodiments of the present invention, the medium in which the biological sample is contained prior to contact with the extraction composition comprises or consists of Hank's balanced salt solution, Universal Transport Medium (UTM), Viral Transport Medium (VTM) or a medium having a total salt concentration in a range +/−30% or +/−20% compared to one or more of the aforementioned media. In embodiments, the medium is a physiological salt solution. The medium comprising the biological sample may be a 0.7% to 1.2% (w/v) or 0.8% to 1% (w/v) alkali metal salt solution. In embodiments, the medium is a 0.9% (w/v) sodium chloride solution. In further embodiments, the medium comprising the biological sample and optionally the virus-deactivating substance is provided by a phosphate buffer, optionally a PBS buffer. As is demonstrated in the examples, the method according to the present invention allows to provide a virus-inactivated biological sample comprised in media for amplification based detection of one or more target nucleic acids without prior nucleic acid purification or removal of the medium. This is highly advantageous, because a robust preparation method is provided that can process biological samples contained in various different media, in particular different media commonly used for receiving, e.g. collecting, respiratory specimens.

According to one embodiment, the medium used for collection comprises at least one virus-deactivating substance as defined above. According to an advantageous embodiment, the composition that is formed when contacting the medium comprising at least one virus-deactivating substance with the biological sample is a composition as disclosed above.

Where the biological sample is comprised in a medium that contains a high amount of salt, the ionic strength of the amplification reaction buffer that is used for setting up the amplification reaction admixture may be reduced to thereby compensate the introduction of ions into the amplification reaction admixture due to the optionally pretreated virus-inactivated biological sample. This embodiment allows to incorporate a high amount of optionally pretreated virus-inactivated biological sample into the amplification reaction admixture (e.g. up to 40%, up to 50% or up to 60% of the total volume of the amplification reaction admixture that comprises all components used in the amplification, which preferably is a reverse transcription amplification) without detrimental inhibition of the amplification reaction by the components that are carried over from the salt-containing medium into the (optionally pretreated) virus-inactivated biological sample and thus the amplification reaction. These advantageous embodiments are disclosed in further detail elsewhere herein. Alternatively, the amount of optionally pretreated virus-inactivated biological sample in the amplification reaction admixture can be reduced to ensure a high performance of the amplification reaction, in particular a reverse transcription amplification reaction.

### Step (B)

As disclosed herein, the method according to the first aspect may comprise (B) pretreating the biological sample. In certain embodiments, pretreatment Step (B) is performed after providing the virus-inactivated biological sample in (A) and prior to (C). In other embodiments, (A) and (B) are performed concurrently.

Such pretreatment step is advantageous, as it can assist the lysis of the biological sample and thus the release of the target nucleic acids to thereby improve the performance of the amplification in (C) and thus the detection of the presence or absence of the target virus(es). Furthermore, pretreatment step (B) can be used to introduce one or more substances that counteract a potentially inhibitory effect of the one or more virus-deactivating substances used for virus inactivation.

In preferred embodiments, pretreating in (B) is performed and comprises
contacting at least an aliquot or all of the virus-inactivated biological sample with an extraction composition preferably comprising
   (a) at least one surfactant,
   (b) at least one nuclease inhibitor, and/or
   (c) at least one reducing agent thereby providing an admixture; and
incubating the admixture to provide the pretreated virus-inactivated biological sample.

The extraction composition may comprise components (a) and (b), components (b) and (c), components (a) and (c) and preferably comprises components (a), (b) and (c). For the detection of RNA viruses, it is preferred to include an RNase inhibitor in order to protect the RNA target nucleic acids from degradation. The extraction composition is preferably an extraction solution.

Suitable extraction compositions/solutions that can be used in pretreatment step (B) and the associated effects are described in detail in EP 20 200 426.3 and EP 20 200 425.5, herein incorporated by reference.

The pretreated virus-inactivated biological sample that is subjected to the amplification reaction may provide at least 20%, at least 30% or at least 40% of the total reaction volume of the amplification reaction. The amount of the pretreated virus-inactivated biological sample that is subjected to the amplification reaction may even provide up to 50% or up to 60% of the total reaction volume of the amplification reaction. That a high amount of the virus-inactivated biological sample can be subjected to the direct amplification without prior nucleic acid purification is advantageous as it increases the sensitivity of the detection reaction.

The extraction composition that is contacted in (B) with the virus-inactivated biological sample can greatly improve the results of the subsequently performed amplification reaction, such as a reverse-transcription amplification reaction. In particular, the extraction composition as used according to the teachings of the present invention can render the target nucleic acids more accessible for the subsequent direct amplification reaction. This by supporting the lysis of the biological sample, including e.g. contained cells and/or virus particles containing the target nucleic acids, whereby the target nucleic acids (if present in the biological sample) are rendered accessible e.g. for the primers and enzymes that are used in the subsequent amplification reaction, which in a preferred embodiment for the detection of RNA viruses is a reverse transcription amplification reaction. At the same time the extraction composition effectively inhibits the undesired degradation of the target nucleic acids by nucleases in the so pretreated virus-inactivated biological sample biological sample. This is particularly advantageous in case of RNA target nucleic acids, because RNA, including viral RNA, is particularly prone to degradation by RNases that are e.g. released from the eukaryotic cells additionally contained in the biological sample or the medium that contains the actual biological sample. It is therefore particularly important to protect the target RNA from degradation during pretreatment/incubation. This particularly if aiming at detecting a pathogenic RNA target nucleic acid (e.g. derived from a RNA virus such as a coronavirus) as there is otherwise a risk of false negative results.

The components comprised in the extraction composition achieve in particular when used in combination the above mentioned beneficial effects in pretreating the virus-inactivated biological sample for direct amplification and advantageously do not interfere with each other or the subsequent direct amplification reaction (which in a preferred embodiment is a reverse transcription amplification reaction). The extraction composition of the present invention that is used in (B) for pretreating the biological sample for direct amplification without prior nucleic acid purification is thus compatible with standard downstream amplification and reverse transcription amplification methods.

As described above, some virus-deactivating substances, such as SDS or povidone iodine, may have an inhibitory effect on the enzyme(s) that are used in (C) in the amplification reaction or reverse transcription amplification reaction, in particular when being used in higher concentrations. As the virus-inactivated biological sample is directly subjected to the amplification reaction (preceded by a reverse transcription reaction in case of RNA targets), without prior nucleic acid purification, it is in such case recommended to first counteract or neutralize the inhibitory effect of the virus-deactivating substance(s) to ensure a high performance of the direct amplification method of the present invention. Suitable substances that can counteract the inhibitory effect of different virus-deactivating substances are described elsewhere herein and are also disclosed in the examples. The use of an extraction solution for pretreatment in (B) advantageously allows to introduce such counteracting substance prior to step (C), whereby the inhibitory effect of the virus-deactivating substance on the enzyme(s) used in (C) is compensated. This allows the preparation of a pretreated virus-inactivated biological sample that is well suitable for direct amplification/reverse transcription amplification in (C) in a rapid manner. The extraction solution can thus serve the purpose to effectively prepare the virus-inactivated biological sample for amplification in (C) and compensate potentially inhibitory effects of the virus-deactivating substance. As discussed herein, the core components of the extraction solution also provide several advantageous functions.

The individual components of the extraction composition and preferred embodiments thereof are described in the following. As disclosed herein, the extraction composition may comprise (a) at least one surfactant, (b) at least one nuclease inhibitor, and (c) optionally at least one reducing agent. The extraction composition is preferably an extraction solution. All disclosures and embodiments described in this application for the extraction composition in general, specifically apply and particularly refer to the preferred embodiment of using an extraction solution even if not explicitly stated.

### The Surfactant Comprised in the Extraction Composition

The extraction composition preferably comprises at least one surfactant. The comprised surfactant supports the lysis of the biological sample, including contained cells and virus particles. The surfactant-induced lysis thereby assists in releasing the target nucleic acids, such as e.g. viral RNA, and thereby renders them accessible for amplification/reverse transcription.

According to one embodiment, the surfactant comprised in the extraction composition counteracts an inhibitory effect of the at least one virus-deactivating substance comprised in the virus-inactivated biological sample on the amplification reaction and/or the reverse transcription reaction performed in (C). As disclosed herein, the surfactant comprised in the extraction composition can counteract inhibitory effects of the virus-deactivating substance. Thereby, the activity of the DNA polymerase and the reverse trancriptase (if used) can be improved in the presence of the virus-deactivating substance exhibiting an inhibitory effect. As is demonstrated in the examples, the activity of the DNA polymerase and the reverse transcriptase can even be restored by including a surfactant that counteracts the inhibitory effect of a surfactant used as virus-deactivating substance. E.g. a cationic surfactant can counteract the inhibitory effect of an anionic surfactant and vice versa. A non-ionic surfactant can likewise counteract the inhibitory effects of an anionic surfactant. Suitable concentrations for the surfactant in the extraction solution to achieve such counteracting or compensating effect can be determined by the skilled person following the guidance in the present description and examples.

The surfactant comprised in the extraction composition is in embodiments selected from non-ionic and zwitterionic surfactants. Suitable non-ionic and zwitterionic surfactants are described elsewhere herein and can also be included in the extraction composition particularly suitable examples are again described in the following.

According to a preferred embodiment, the surfactant comprised in the extraction solution is a non-ionic surfactant. Non-ionic surfactants are particularly suitable to prepare the virus-inactivated biological sample for direct amplification in (C). Furthermore, and as also demonstrated in the examples, non-ionic surfactants can compensate inhibitory effects of virus-deactivating substances, such as e.g. anionic surfactants. Hence, the use of a non-ionic surfactant is advantageous as it can effectively support the lysis of the biological sample, can counteract inhibitory effects of various virus-deactivating substances and usually exerts no or only a low inhibitory effect on the amplification/reverse transcription amplification even at higher concentrations as is demonstrated in the examples.

According to one embodiment, the non-ionic surfactant is a polyoxyethylene-based non-ionic surfactant. It may be selected from the group consisting of polyoxyethylene fatty acid esters, in particular polyoxyethylene sorbitan fatty acid esters; polyoxyethylene fatty alcohol ethers; polyoxyethylene alkylphenyl ethers; and polyoxyethylene-polyoxypropylene block copolymers. In advantageous embodiments, the non-ionic surfactant comprised in the extraction composition is selected from polyoxyethylene fatty acid esters, in particular polyoxyethylene sorbitan fatty acid esters, and polyoxyethylene fatty alcohol ethers.

According to one embodiment, the extraction composition comprises a polyoxyethylene fatty acid ester, comprising
a fatty acid derived from laureate, palmitate, stearate and oleate,
a polyoxyethylene component containing from 2 to 150, 4 to 100, 6 to 50 or 6 to 30 (CH₂CH₂O) units.

Polyoxyethylene sorbitan fatty acid esters, also referred to as polysorbates, are particularly preferred as non-ionic surfactant for the extraction composition. The non-ionic surfactant may be selected from polyoxyethylene (20) sorbitan monolaurate (e.g. Tween20), polyoxyethylene (4) sorbitan monolaurate (e.g. Tween21), polyoxyethylene (40) sorbitan monopalmitate (e.g. Tween40), polyoxyethylene (60) sorbitan monostearate (e.g. Tween60), polyoxyethylene (4) sorbitan monostearate (e.g. Tween61), polyoxyethylene (20) sorbitan tristearate (e.g. Tween65), polyoxyethylene (40) sorbitan monooleate (e.g. Tween80), polyoxyethylene (5) sorbitan monooleate (e.g. Tween81), polyoxyethylene sorbitan trioleate (e.g. Tween85), and polyoxyethylen (20) sorbitan monoisostearate. According to a preferred embodiment, the polyoxyethylene fatty acid ester is selected from polysorbate 20, polysorbate 40, polysorbate 60 and polysorbate 80. As is demonstrated in the examples, such non-ionic surfactants are advantageous because they assist the lysis and thus preparation of the biological sample for direct amplification and do not inhibit the subsequent amplification reaction (PCR and RT-PCR) even when used in higher concentrations. The use of polysorbate 20 is particularly preferred.

According to one embodiment the extraction composition comprises as non-ionic surfactant a polyoxyethylene fatty alcohol ether, comprising
a fatty alcohol component having from 6 to 22 carbon atoms, and
a polyoxyethylene component containing from 2 to 150, 4 to 100, 6 to 50 or 6 to 30 (CH₂CH₂O) units.

The polyoxyethylene fatty alcohol ether may be selected from the group consisting of polyoxyethylene lauryl ether, polyoxyethylene cetyl ether, polyoxyethylene stearyl ether and polyoxyethylene oleyl ether. In a preferred embodiment, the polyoxyethylene fatty alcohol ether is selected from the group comprising polyoxyethylene cetyl ether, polyoxyethylene stearyl ether and/or polyoxyethylene oleyl ether. Suitable examples include but are not limited to polyoxyethylene cetyl or polyoxyethylene oleyl alcohol ethers, such as polyoxyethylene(10) cetyl ether (Brij® 56), polyoxyethylene(20) cetyl ether (Brij® 58) and polyoxyethylene(20) oleyl ether (Brij® 98).

In one embodiment the extraction composition comprises as non-ionic surfactant a polyoxyethylene alkyl phenyl ether. The polyoxyethylene alkylphenyl ether may have an alkyl group containing from five to 15 carbon atoms, such as 6 to 10 carbon atoms. Also encompassed are branched or unbranched C₈- to C₁₀-alkyl groups, such as branched or unbranched C₈- and C₉-alkyl groups, e.g. isooctyl groups and nonyl groups. The polyoxyethylene alkylphenyl ether may be selected from the group consisting of polyoxyethylene nonylphenyl ether and polyoxyethylene isooctylphenyl ether. It may be Triton X-100.

Polyoxyethylene-polyoxypropylene block copolymers may also be included as non-ionic surfactant in the extraction composition. Polyoxyethylene-polyoxypropylene block copolymers are also referred to as “poloxamers”. Polyoxyethylene-polyoxypropylene block copolymers may be of the empirical formula HO(C₂H₄O)ₐ(C₃H₆O)_{b}(C₂H₄O)ₐH, where “a” refers to the number of polyoxyethylene units and “b” refers to the number of polyoxypropylene units, with the a/b weight ratio optionally being in the range from 0.1 to 3. Such polyoxyethylene-polyoxypropylene block copolymers can be obtained, for example, under the trade name Pluronic® or Synperonic®. Further non-ionic surfactants were also described above in conjunction with the virus-deactivating substance and it is referred to the respective disclosure which also applies here.

According to a further embodiment, the surfactant is a zwitterionic surfactant. The zwitterionic surfactant may be a betaine such as N,N,N trimethylglycine. In one embodiment the extraction solution comprises the zwitterionic surfactant such as a betaine in a concentration lies in the range of 50 mM to 1M, such as 100 mM-500 mM.

According to one embodiment, the virus-deactivating substance differs from the surfactant comprised in the extraction buffer.

According to one embodiment, the crude biological sample is contacted with an extraction solution that comprises the surfactant, preferably a non-ionic surfactant, in a concentration that lies in a range of 0.1% to 30% (w/v). Suitable ranges include but are not limited to 0.5% to 25% (w/v), 0.7% to 20% (w/v) and 1% to 15% (w/v). In further embodiments the surfactant concentration in the extraction solution is 1.2% to 10% (w/v), 1.5% to 8% (w/v) or 2% to 5% (w/v). Following the teachings of the present application, the skilled person can chose suitable surfactant concentrations for the extraction solution also depending on the amount of virus-inactivated biological sample to be contacted with the extraction solution. To ensure that the pretreated virus-inactivated biological sample comprises a high amount of the original biological sample (which is optionally contained in medium) and avoid unnecessary dilutions of the virus-inactivated biological sample it is advantageous to use a concentrated extraction solution. This allows using a small amount of extraction solution in order to prepare a larger amount of biological sample. E.g. the extraction solution may be concentrated 3×, 4× or 5×. The above mentioned concentrations are suitable for providing such concentrated extraction compositions. In further embodiments, the extraction solution is concentrated 10×, 15× or 20×.

In advantageous embodiments, the resulting admixture that is prepared by contacting the (virus-inactivated) biological sample (optionally contained in medium) with the extraction composition comprises the surfactant originating from the extraction composition, preferably a non-ionic surfactant, in a concentration that lies in a range of 0.075% to 20% (w/v). Suitable final concentration ranges for the surfactant, preferably a non-ionic surfactant, in the prepared admixture include but are not limited to 0.1% to 15% (w/v), 0.15% to 15% (w/v), 0.2% to 10% (w/v) and 0.25% to 8% (w/v). In further embodiments, the final surfactant concentration in the prepared admixture is 0.2% to 5% (w/v), 0.25% to 3% (w/v) or 0.3% to 2% (w/v).

The extraction solution may also comprise two or more surfactants, e.g. selected from non-ionic surfactants and zwitterionic surfactants.

### The Nuclease Inhibitor Comprised in the Extraction Composition

The extraction composition preferably supports the lysis of the biological sample, including contained viral particles, and at the same time inhibits nucleases that could degrade the target nucleic acids, such as target RNA or target DNA. It was found that biological samples used for virus testing, such as swab samples comprised in transport media, often contain high amounts of nucleases. The nucleases may origin from the comprised eukaryotic cells but also from undefined media components, such as fetal calf serum that may be comprised in standard swab transport medium.

To inhibit the degradation of target nucleic acids by nucleases in the pretreated virus-inactivated biological sample, the extraction composition preferably comprises at least one nuclease inhibitor. It is preferred that the nuclease inhibitor achieves strong nuclease inhibition, in order to effectively protect the target nucleic acids that are released e.g. during pretreatment from nuclease degradation. As disclosed herein, nucleases may also be released from the cells that are lysed by the extraction composition.

The nuclease inhibitor may be an RNase inhibitor or a DNase inhibitor. The extraction solution comprises a nuclease inhibitor that is capable of protecting the target nucleic acid of interest and thus the target nucleic acids that are used for determining whether the virus is present or absent. The extraction composition may also comprise two or more nuclease inhibitors, such as (i) two or more RNase inhibitors, (ii) two or more DNase inhibitors or (iii) one or more RNase inhibitors and one or more DNase inhibitors. Using an extraction composition comprising a RNase inhibitor as well as a DNase inhibitor can be advantageous in order to provide an universal extraction composition and thus universal preparation method that is compatible with RNA and DNA target nucleic acids and thus is suitable for the detection of RNA and DNA viruses.

The nuclease inhibitor that is comprised in the extraction composition does not interfere with the subsequent enzymatic reaction (such as the amplification and/or reverse amplification) at least in the concentration in which it is included into the enzymatic reaction via the pretreated virus-inactivated biological sample that comprises the biological sample and components of the extraction composition. Hence, a reverse transcription reaction and/or an amplification reaction can be performed in the presence of the comprised nuclease inhibitor.

According to one embodiment, the nuclease inhibitor is a RNAase inhibitor. Incorporating a RNase inhibitor into the extraction composition that is used for pretreating the virus-inactivated biological sample greatly improves the results of a subsequently performed direct reverse transcription amplification to which the optionally pretreated virus-inactivated biological sample is subjected without prior nucleic acid purification. As disclosed herein, the target nucleic acid is in preferred embodiments a RNA, such as a viral RNA. Therefore, preventing degradation of the viral RNA is particularly advantageous to increase the sensitivity of the virus detection. The RNase inhibitor may have broad-spectrum RNase inhibitory properties and may inhibit RNase A, B and C as well as human placental RNase. It does not inhibit the reverse transcriptase or the DNA polymerase used, such as Taq polymerase.

The use of a strong RNase inhibitor is preferred in order to maximize the protection of the target RNA from degradation. Strong RNase inhibitors are well-known and are often provided by proteins, in particularly recombinantly produced proteinaceous RNase inhibitors. In an advantageous embodiment, the RNase inhibitor is thus a proteinaceous RNase inhibitor. Numerous proteinaceous RNase inhibitors are commercially available and can thus be used in conjunction with the present invention as is also demonstrated in the examples. Examples of proteinaceous RNase inhibitors include, but are not limited to, QIAGEN RNase Inhibitor, RNasin® Ribonuclease Inhibitor, NxGen RNase inhibitor and others.

The amount/concentration of the RNase inhibitor in the extraction composition of the present invention can be experimentally determined by the skilled person following the guidance provided in the application and e.g. the manufacturer's instructions for the chosen RNase inhibitor. Incorporating more of the RNase inhibitor will usually achieve a stronger RNase inhibitory effect.

In one embodiment, where RNA target nucleic acids are of interest, the extraction composition comprises a RNase inhibitor, preferably a proteinaceous RNase inhibitor, but does not comprise a separate DNase inhibitor. In this embodiment, the RNA target nucleic acids are protected from degradation by the RNase inhibitor, while any degradation of non-target DNA would reduce the non-target nucleic acid background. Corresponding considerations apply where DNA target nucleic acids are of interest, and wherein the extraction composition comprises a DNase inhibitor but does not comprise a separate RNase inhibitor.

### The Reducing Agent Comprised in the Extraction Composition

In a preferred embodiment, the extraction composition that is used in (B) for pretreating the virus-inactivated biological sample comprises a reducing agent. Incorporating a reducing agent is advantageous as it assists the pretreatment of the virus-inactivated biological sample for direct amplification.

The reducing agent preferably supports the destruction of disulfide bonds and denaturation of proteins comprised in the biological sample. The reducing agent can thus assist in the inhibition of the nucleases. It can furthermore support the release of the target nucleic acids. Furthermore, incorporating a reducing agent into the extraction composition is advantageous because it can assist to liquefy the biological sample. This can simplify the processing of viscous biological samples, such as respiratory samples. Liquefying a viscous biological sample is advantageous because the target nucleic acids are better accessible in a liquefied biological sample and the optionally pretreated virus-inactivated biological sample is more homogeneous. Reducing agents are known in the art. The reducing agent that is comprised in the extraction composition does not interfere with the subsequent enzymatic reaction (such as the amplification and/or reverse amplification) at least in the concentration in which it is included into the enzymatic reaction via the pretreated virus-inactivated biological sample that comprises the biological sample, the at least one virus-deactivating substance and components of the extraction composition. Hence, a reverse transcription reaction and/or an amplification reaction can be performed in the presence of the comprised reducing agent respectively the pretreated virus-inactivated biological sample that comprises the reducing agent.

According to one embodiment, the reducing agent comprised in the extraction composition counteracts an inhibitory effect of the at least one virus-deactivating substance comprised in the virus-inactivated biological sample on the amplification reaction and/or the reverse transcription reaction performed in (C). As is demonstrated in the examples, the reducing agent comprised in the extraction composition/extraction solution can also compensate inhibitory effects of virus-deactivating substances, such as disinfectants. E.g. oxidizing agents such as povidone iodine can exert an inhibitory effect at higher concentrations. This inhibitory effect of oxidizing agents such as povidone iodine can be compensated by adding a suitable amount of a reducing agent. Advantageously, the reducing agent can be directly included into the extraction composition used for pretreatment, thereby saving handling steps.

In one embodiment, the reducing agent is selected from Tris(carboxyethyl)phosphine (TCEP), Dithiothreitol (DTT), N-acetyl cysteine, THPP (Tris(hydroxypropyl)phosphine), 1-thioglycerol and beta-mercaptoethanol. In one embodiment, the comprised reducing agent is selected from Tris(carboxyethyl)phosphine (TCEP), Dithiothreitol (DTT), N-acetyl cysteine, THPP (Tris(hydroxypropyl)phosphine) and 1-thioglycerol. In one embodiment, the comprised reducing agent is selected from Tris(carboxyethyl)phosphine (TCEP), Dithiothreitol (DTT) and N-acetyl cysteine. These reducing agents do not interfere with the subsequent reverse transcription reaction and/or an amplification reaction when included in appropriate concentrations. In a particular embodiment, the extraction composition comprises Tris(carboxyethyl)phosphine (TCEP) as reducing agent. TCEP is storage stable and therefore, is advantageous for ready-to-use kit formats.

An extraction composition comprising in addition to the RNase inhibitor and surfactant a reducing agent such as TCEP can further improve the subsequently performed direct amplification reaction to which the pretreated virus-inactivated biological sample is subjected.

In one embodiment, the extraction composition comprises the reducing agent in a concentration that lies in a range of 0.3 mM to 50 mM. Suitable concentration ranges for the reducing agent in the extraction composition include but are not limited to 0.5 mM to 25 mM, 1 mM to 20 mM and 1.5 mM to 15 mM. In embodiments, the extraction composition comprises the reducing agent in a concentration in a range of 2 mM to 10 mM or 2 mM to 5 mM. The extraction composition comprises in one embodiment a reducing agent that is selected from Tris(carboxyethyl)phosphine (TCEP), Dithiothreitol (DTT), N-acetyl cysteine, THPP (Tris(hydroxypropyl)phosphine) and 1-thioglycerol in a concentration that lies in the range of 1 mM to 10 mM or 2 mM to 5 mM, wherein in a preferred embodiment the reducing agent is TCEP. Following the teachings of the present application, the skilled person can chose a suitable reducing agent concentration for the extraction composition. E.g. in case of more viscous biological samples, the concentration may be increased to further support the rapid liquefaction of the biological sample, also when the biological sample is contained in medium. As noted above, to ensure that the pretreated virus-inactivated biological sample comprises a high amount of the original biological sample (which is optionally contained in medium) it is advantageous to use a concentrated extraction solution. This allows using a small amount of extraction solution in order to prepare a larger amount of biological sample. E.g. the extraction solution may be concentrated 3×, 4× or 5×. The above mentioned concentrations are suitable for providing such concentrated extraction compositions. In further embodiments, the extraction solution is concentrated 10×, 15× or 20×. Thus, the extraction solution may be concentrated in a range of 3× to 20×.

In advantageous embodiments, the resulting admixture that is prepared by contacting the (virus-inactivated) biological sample (optionally contained in medium) with the extraction composition comprises the reducing agent in a concentration that lies in a range of 0.1 mM to 15 mM. Suitable concentration ranges for a reducing agent such as TCEP in the prepared admixture include but are not limited to 0.2 mM to 10 mM, 0.25 mM to 8 mM and 0.3 mM to 5 mM. In further embodiments, the final reducing agent concentration in the prepared admixture is 0.35 mM to 2 mM or 0.4 mM to 1.5 mM.

According to an advantageous embodiment, the extraction composition comprises a reducing agent selected from Tris(carboxyethyl)phosphine (TCEP), Dithiothreitol (DTT), N-acetyl cysteine, THPP (Tris(hydroxypropyl)phosphine) and 1-thioglycerol in a concentration that lies in the range of 1 mM to 10 mM or 2 mM to 5 mM.

### Embodiments of the Extraction Composition

As noted above, in preferred embodiments the extraction composition is provided as liquid composition. The use of an extraction solution in (B) is advantageous because such solution can be easily mixed with the biological sample, which in preferred embodiments is a biological sample comprised in a composition that further comprises a virus-deactivating substance and medium. The active components of the extraction solution, i.e. the nuclease inhibitor (preferably a proteinaceous RNase inhibitor), the surfactant (preferably a non-ionic surfactant) and the preferably comprised reducing agent, can be quickly dispersed in the biological sample and can thereby ensure the efficient lysis and pretreatment of the biological sample and protection of the target nucleic acids. This process can be assisted by agitation, such as vortexing, to ensure that the extraction solution and the biological sample are mixed well.

Particularly advantageous extraction solutions suitable to pretreat a (virus-inactivated) biological sample such as a biological sample contained in medium for direct reverse transcription and amplification of the target nucleic acids without prior purification of the contained nucleic acids are described in the following. As is demonstrated by the examples, accordingly designed extraction solutions achieve particularly favorable results. In embodiments, the subsequently described extraction solutions consist essentially of or consist of the carrier liquid (which may comprise a buffering agent or can be unbuffered) of the extraction solution and the identified active ingredients.

According to one embodiment, the extraction composition, which preferably is a liquid composition, comprises
(a) at least one non-ionic surfactant,
(b) at least one proteinaceous RNase inhibitor, and
(c) at least one reducing agent.

Suitable and preferred embodiments of the non-ionic surfactant and the reducing agent were described above and it is referred to the respective disclosure.

According to one embodiment, the extraction composition, which preferably is a liquid composition, comprises
(a) at least one polyoxyethylene-based non-ionic surfactant,
(b) at least one proteinaceous RNase inhibitor, and
(c) at least one reducing agent selected from Tris(carboxyethyl)phosphine (TCEP), Dithiothreitol (DTT), N-acetyl cysteine, THPP (Tris(hydroxypropyl)phosphine) and 1-thioglycerol.

As disclosed herein, the active ingredients of the extraction composition, which preferably is a liquid composition, may consist essentially of or may consist of
(a) a non-ionic surfactant, preferably a polyoxyethylene-based non-ionic surfactant,
(b) a proteinaceous RNase inhibitor, and
(c) a reducing agent, preferably selected from Tris(carboxyethyl)phosphine (TCEP), Dithiothreitol (DTT), N-acetyl cysteine, THPP (Tris(hydroxypropyl)phosphine) and 1-thioglycerol.

Suitable and preferred embodiments of the one polyoxyethylene-based non-ionic surfactant were described above and it is referred to the respective disclosure. In advantageous embodiments, the non-ionic surfactant comprised in the extraction solution is selected from polyoxyethylene fatty acid esters, in particular polyoxyethylene sorbitan fatty acid esters, and polyoxyethylene fatty alcohol ethers.

According to a preferred embodiment, the extraction composition, which preferably is a liquid composition, comprises
(a) at least one polysorbate,
(b) at least one proteinaceous RNase inhibitor, and
(c) Tris(carboxyethyl)phosphine (TCEP).

Such extraction composition is very advantageous and allows to pretreat even difficult biological samples, including respiratory specimens comprised in medium, for direct reverse transcription and amplification of comprised RNA target nucleic acids (such as viral RNA targets) with favorable sensitivity. Suitable polysorbates that can be included as non-ionic surfactant are disclosed above and it is referred to the respective disclosure. As described, the polysorbate may be selected from polysorbate 20, polysorbate 40, polysorbate 60 and polysorbate 80. Polysorbate 20 is a particularly preferred polysorbate that can be included in the extraction solution as non-ionic surfactant. In one embodiment, the active ingredients of the extraction solution may consist essentially of or may consist of
(a) at least one polysorbate,
(b) at least one proteinaceous RNase inhibitor, and
(c) Tris(carboxyethyl)phosphine (TCEP).

The extraction composition may have a pH in the range of 6.0 to 9.0, such as 6.0 to 8.5 or 6.3 to 8.0. The pH may be in the range of 6.5 to 7.5, such as about 7.0. The extraction composition may comprise a buffer or may be unbuffered.

As no purification occurs prior to amplification in the rapid method according to the invention, the extraction composition preferably does not comprise any components that substantially interfere with the subsequent direct amplification. While such inhibitory effects may potentially be counteracted using the technology described herein, it is preferred to avoid such interfering substances, where possible. The same applies to the medium that may be used for collecting/storing/transporting the biological sample. Suitable embodiments are described herein.

As noted, the extraction composition preferably does not comprise ingredients in a concentration that could inhibit the subsequently performed amplification/reverse transcription amplification of the one or more target nucleic acids when the pretreated virus-inactivated biological sample, that comprises the components of the extraction composition, is in (C) subjected to the amplification reaction/reverse transcription amplification reaction. Furthermore, the extraction composition should not comprise ingredients that counteract or damage the comprised core components, i.e. the surfactant, the nuclease inhibitor and, if comprised, the reducing agent. It is thus advantageous if the extraction composition/extraction solution does not comprise one or more, two or more, three or more or all of the following components:
an ionic surfactant if a non-ionic surfactant is comprised;
a chaotropic salt;
chloride ions in a concentration exceeding 10 mM, wherein preferably the extraction composition does not comprise chloride ions;
an aliphatic C1-C5 alcohol; and/or
a proteinase enzyme (or if such enzyme is used, it should be inactivated prior to performing the amplification reaction to avoid damage of the enzyme(s) used in the amplification).

The components of the extraction composition/extraction solution are comprised in the pretreated biological sample and are thus transferred to the subsequent amplification reaction, which preferably is a reverse transcription amplification reaction. It is therefore advantageous to design the extraction solution as simple as possible. In preferred embodiments, the active ingredients of the extraction composition, respectively the extraction solution, therefore consist essentially of or consist of (a) a surfactant, preferably a non-ionic surfactant, (b) the nuclease inhibitor and (c) the reducing agent, if comprised. For pretreating virus-inactivated biological samples for a subsequently performed direct reverse transcription amplification reaction (without prior nucleic acid purification) the nuclease inhibitor is as described herein a RNase inhibitor, preferably a proteinaceous RNase inhibitor.

The core components comprised in the extraction composition of the present invention are not harmful and may even support or promote the performance of the amplification such as the reverse transcription amplification. Advantageously, the extraction composition may be free of anorganic salts, in particular chloride salts and alkali metal salts. This is advantageous as the ionic strength due to comprised salts is not further increased in the pretreated virus-inactivated biological sample due to the extraction composition. This is advantageous when aiming at processing biological samples comprised in salt-containing medium (e.g. swab samples comprised in common transport media).

### Further Features and Embodiments of (B)

The admixture that is provided by contacting the virus-inactivated biological sample with the extraction composition according to the present invention is preferably incubated so that the ingredients comprised in the extraction composition can digest the biological sample, while protecting the target nucleic acids. In embodiments, incubation occurs for 1 to 60 min, 1 to 30 min or 1 to 20 min. In further embodiments, incubation occurs for 1 to 15 min or 1 to 10 min. The extraction composition according to the present invention is highly effective, so that very short incubation times of 1.5 to 5 min or 1.5 to 3 min, such as 2 min, are sufficient in order to pretreat a virus-inactivated biological sample for direct amplification. This is highly advantageous because it significantly shortens the processing time compared to workflows that require nucleic acid purification prior to amplification or incorporate other more time consuming preparation steps. However, also longer incubation times are feasible without compromising the quality of the target nucleic acids because target degradation is effectively reduced with the extraction composition of the present invention. This is advantageous where a high number of biological samples are processed in parallel. The biological samples that are first contacted with the extraction composition may simply be incubated for a longer time without compromising the quality of the pretreated virus-inactivated biological sample until the last biological samples were also contacted with the extraction composition and incubated for an appropriate time. This pretreatment protocol of the present invention is therefore very robust and ensures uniform results even if the incubation time varies between pretreated virus-inactivated biological samples.

Preparation of the admixture comprising the virus-inactivated biological sample and the extraction composition may comprise agitating the biological sample in contact with the extraction composition to ensure a thorough admixture of the virus-inactivated biological sample and the extraction composition. For agitation, the admixture may e.g. be aspirated and dispensed and/or vortexted.

Advantageously, the steps of contacting the biological sample with the extraction composition and incubating the admixture may be carried out at ambient temperature (e.g. room temperature). In embodiments, all preparation steps apart from the enzymatic reaction (amplification or reverse-transcription amplification) are carried out at ambient temperature. This simplifies the performance of the method according to the present invention. If desired, these steps may also be carried out on ice.

In certain embodiments, pretreatment step (B) is performed but does not involve heating the biological sample in contact with the extraction composition to a temperature 75° C., such as 70° C., 65° C., 60° C., 55° C., 50° C., 45° C. or 40° C. for at least 2 min prior to subjecting at least an aliquot or all of the pretreated virus-inactivated biological sample to the amplification reaction. Such heating step may reduce the performance of the pretreated virus-inactivated biological sample in the subsequent amplification/reverse transcription amplification reaction and is therefore preferably avoided. In particular, the method should not involve heating the biological sample in contact with the extraction composition to a temperature that would denature a comprised proteinaceous RNase inhibitor prior to subjecting at least an aliquot or all of the pretreated virus-inactivated biological sample to the amplification reaction. As disclosed herein, such strong proteinaceous RNase inhibitor is particularly advantageous in order to protect the labile RNA targets from degradation during preparation of the biological sample for direct amplification based detection of the target nucleic acid. Therefore, heating steps that would denature the proteinaceous RNase inhibitor should be avoided to ensure the correct performance of the extraction composition. In particularly preferred embodiments, the admixture comprising the virus-inactivated biological sample and the extraction composition is not heated prior to subjecting at least an aliquot or all of the pretreated virus-inactivated biological sample to the subsequent enzymatic reaction selected from reverse transcription amplification and amplification. After subjecting the pretreated virus-inactivated biological sample to the enzymatic reaction, heating steps may of course be performed and are usually performed to establish e.g. the conditions for the reverse transcription reaction and/or amplification reaction and for activating “hot start” applications.

The method according to the present invention provides a rapid and simple workflow that does not require elaborate pretreatment steps. Contacting the virus-inactivated biological sample with the extraction composition and shortly incubating the admixture is advantageous and sufficient to provide the pretreated virus-inactivated biological sample that enables a direct amplification of the target nucleic acids without prior nucleic acid purification.

Therefore, in preferred embodiments, the method according to the first aspect does not involve centrifuging the optionally pretreated virus-inactivated biological sample prior to subjecting at least an aliquot or all of the optionally pretreated virus-inactivated biological sample to the amplification reaction. In particular, no centrifugation steps are required to remove components (e.g. cellular debris) from the optionally pretreated virus-inactivated biological sample prior to subjecting at least an aliquot or all of the optionally pretreated virus-inactivated biological sample to the amplification reaction. If desired, a brief centrifugation step may be included in order to e.g. collect liquid at the bottom of the reaction vessel, e.g. after contacting the optionally pretreated virus-inactivated biological sample with the components necessary for performing the reverse-transcription amplification or amplification as it is also described in the examples.

Advantageously, the method according to the first aspect can be performed so that it does not involve removing cellular components from the optionally pretreated virus-inactivated biological sample prior to subjecting at least an aliquot or all of the optionally pretreated virus-inactivated biological sample to the amplification reaction. As disclosed herein, the methods according to the present invention furthermore do not require purifying the target nucleic acid prior to subjecting at least an aliquot or all of the optionally pretreated virus-inactivated biological sample to the amplification reaction. This significantly simplifies and streamlines the workflow.

Performing a Heating Step Prior to Contact with the Extraction Composition

According to one embodiment, the biological sample is heated prior to optionally contacting the virus-inactivated biological sample in (B) with the extraction composition to improve pathogen inactivation. As disclosed herein, the biological sample may be comprised in a medium (such as a transport medium described herein). To include one or more heating steps for pathogen inactivation can assist biosafety and biosecurity because heat-inactivating pathogens potentially comprised in the biological sample further reduces the infection risk during sample handling and allows to simplify processing.

According to one embodiment, the method according to the first aspect comprises heating the biological sample prior to contact with a virus-deactivating substance. According to another embodiment, the method according to the invention comprises heating the composition comprising the biological sample and the virus-deactivating substance. According to a further embodiment, heating the biological sample is performed prior to and after contacting the biological sample with at least one virus-deactivating substance. Heating is performed at a temperature suitable to inactivate pathogens, including viruses, prior to contacting the obtained virus-inactivated biological sample with the extraction composition in (B). Heating to assist the inactivation of pathogens such as viruses may comprise heating the biological sample to a temperature ≥50° C., ≥55° C. or ≥60° C. Such heating protocols for pathogen inactivation are known in the art. Heating temperatures at the lower end usually require longer heating times for pathogen inactivation, such as virus inactivation. In preferred embodiments heating is performed at a temperature ≥75° C., ≥80° C. or ≥85° C., preferably ≥90° C. or ≥95° C. Heating at such higher temperatures is advantageous as the heating period necessary to achieve pathogen inactivation can be shorter, allowing the use of short heating times for pathogen inactivation. Furthermore, the use of such higher heating temperatures for pathogen inactivation may also denature proteins comprised in the biological sample that could negatively affect the comprised target nucleic acids. As noted, any such heating step is performed prior to optional pretreatment step (B) as heating in the presence of the extraction composition prior to step (C) should be avoided.

Previous studies investigated the influence of an initial heat inactivation step on the PCR sensitivity and accuracy. These studies consistently confirmed that heating of the samples prior to SARS-CoV-2 detection by PCR results in dramatically increased Ct values and thus in decreased detection rates independent of whether viral nucleic acids were extracted subsequent to the heating step or not and also independent of the assay used (E, N, ORF1 ab) (Zou et al., 2020; Alcoba-Florez et al., 2020; Fomsgaard & Rosenstierne, 2020). However, these drawbacks of prior art methods can be overcome by the method of the present invention, wherein after heating, the virus-inactivated biological sample is pretreated with an extraction composition as disclosed herein. Heating the biological sample in advance and in the absence of the extraction solution can assist pathogen inactivation and the following addition of the extraction composition of the present invention, preferably comprising (a) a non-ionic surfactant, (b) a nuclease inhibitor, preferably a proteinaceous RNase inhibitor in case of RNA targets and (c) a reducing agent, to the virus-inactivated biological sample prevents the subsequent degradation of the target nucleic acid due to inhibition of the RNases thereby providing improved results without impairing signal intensity in the subsequent amplification. These beneficial effects are not seen with prior art heating procedures which report a decrease of the signal intensity as described above.

After heating, the virus-inactivated biological sample may be contacted within ≤2 h, ≤1 h, ≤0.5 h, ≤20 min or ≤15 min with the extraction composition for sample pretreatment. Therefore, the virus-inactivated biological samples may be directly further processed by contacting the heated biological sample with the extraction composition, if desired. Optionally, a cooling step can be performed in-between heating and contacting the virus-inactivated biological sample with the extraction composition.

Contacting the virus-inactivated biological sample with the extraction composition may also be delayed. Therefore, after heating the virus-inactivated biological sample may be put on hold, stored or transported prior to contacting the virus-inactivated biological sample with the extraction composition of the present invention. Short—as well as long-term storage of the virus-inactivated biological sample prior to contact with the extraction composition is possible. According to one embodiment, the time span between heating the biological sample for providing the virus-inactivated biological sample and contacting the obtained virus-inactivated biological sample with the extraction composition is ≥2 h. In embodiments, the time-span is within a range of ≥2 h and ≤150 h, ≥3 h and ≤100 h or ≥4 h and ≤75 h. In further embodiments, the time-span is at least 12 h, at least 24 h and may be at least 2 days or at least 3 days.

According to one embodiment, virus inactivation is assisted by heating and the method comprises heating the biological sample in the collection container used for receiving the collected biological sample. The biological sample may be comprised in a medium in the collection container. Advantageously, the medium may comprise a virus-deactivating substance as disclosed herein. In one embodiment, the collection container has not been opened after collection of the biological sample and prior to heating for inactivating pathogens potentially comprised in the biological sample. In a further embodiment, an aliquot of the biological sample comprised in medium is obtained and heated for pathogen inactivation as described herein prior to contacting the virus-inactivated biological sample with the extraction composition.

In embodiments, no heating step is performed for virus, respectively pathogen, inactivation in the method according to the first aspect.

### Performing Steps (A) and (B) Concurrently or Sequentially

In certain embodiments, virus-inactivation (A) and pretreatment (B) are performed concurrently in order to provide a pretreated virus-inactivated biological sample. Hence, virus-inactivation (A) and pretreatment (B) can be performed essentially at the same time in one process step. Such embodiment can reduce the processing time to obtain the pretreated virus-inactivated biological sample. E.g. after arrival of the collected biological sample, the biological sample may be contacted with the virus-deactivating substance and an extraction composition. Contacting may occur in any order and the virus-deactivating substance may also be directly comprised in the extraction composition. The provided composition/admixture comprises the biological sample (optionally contained in medium used for storage/transport), the virus-deactivating substance and the extraction composition and is incubated. During incubation, virus inactivation and pretreatment can occur together and thus essentially at the same time.

According to one embodiment, the virus-deactivating substance, which optionally is a surfactant, is included in the extraction composition which is contacted with the biological sample that is optionally comprised in medium. The extraction composition comprises in this embodiment at least one virus-deactivating substance and additionally
(a) at least one surfactant,
(b) at least one nuclease inhibitor, and/or
(c) at least one reducing agent.

As disclosed above, it is advantageous to include a nuclease inhibitor, in particular a strong RNase inhibitor if detecting an RNA viruses. Therefore, the extraction composition preferably comprises at least component (b). If the virus-deactivating substance is a surfactant, the use of an additional surfactant is optional. If a further surfactant is used, it should be chosen such that it does not counteract the virus-deactivating activity of the virus-deactivating surfactant to ensure effective virus inactivation. The extraction composition may also comprise two virus-deactivating surfactants, e.g. of different virucidal strength. In embodiments, a cationic, zwitterionic or anionic surfactant, preferably a cationic surfactant (such as a quaternary ammonium salt), is used as virus-deactivating substance. A non-ionic surfactant may be included as component (a).

The extraction composition comprising the virus-deactivating substance may furthermore comprise a reducing agent. If a reducing agent is included, the virus-deactivating substance is preferably not an oxidizing agent as the reducing agent would neutralize the activity of the oxidizing agent as is demonstrated in the examples. In embodiments, the extraction composition comprises a virus-deactivating surfactant and (a) optionally an additional surfactant; (b) a nuclease inhibitor, preferably a RNase inhibitor and (c) a reducing agent.

Where the extraction composition comprises a virus-deactivating substance in a concentration where it could exert an inhibitory effect on the amplification and/or reverse transcription in (C), a counteracting substance may be added prior or during (C). E.g. such counteracting substance may be included in the reaction mixture prepared in (C) for performing the amplification and/or reverse transcription.

Suitable embodiments for the individual components of the extraction composition including concentrations in the extraction composition and the resulting admixture and for the virus-deactivating substance, including concentrations in the composition comprising the biological sample and the virus-deactivating substance are described in detail elsewhere herein and it is referred to the respective disclosure which also applies here.

The biological sample may also be contacted with an extraction composition as disclosed herein prior to contacting the resulting admixture with a virus-deactivating substance to provide a pretreated virus-inactivated biological sample. Furthermore, pretreatment step (B) may also be performed in the presence of some or all components necessary for performing the amplification/reverse transcription amplification in (C). After incubation for pretreatment the prepared admixture is then subjected to the actual amplification reaction, e.g. by transfer to a thermocycler. In one embodiment that is also illustrated in the examples, pretreatment step (B) is performed after virus inactivation in step (A) and prior to step (C).

### Step (C)

Step (C) comprises subjecting at least an aliquot or all of the optionally pretreated virus-inactivated biological sample to an amplification reaction and amplifying the at least one target nucleic acid, optionally wherein a reverse transcription reaction is performed in order to reverse transcribe RNA to cDNA prior to amplification. As disclosed herein, a reverse transcription reaction and/or an amplification reaction, such as a reverse-transcription and amplification reaction, preferably a quantitative RT-PCR can be performed using the optionally pretreated virus-inactivated biological sample. As is demonstrated by the examples, the technology of the invention provides a (optionally pretreated) virus-inactivated biological sample that is directly suitable for amplification based detection of one or more target nucleic acids, such as RNA target nucleic acids, for virus detection while ensuring a good performance and sensitivity. Details for the target viruses/target nucleic acids as well as suitable and preferred embodiments for step (C) are described in the following.

### The Target Nucleic Acids and Target Viruses to be Detected

As disclosed herein, the technology of the present invention allows the detection of the presence or absence of at least one virus in the biological sample, based on the amplification based detection of at least one target nucleic acid that is derived from and thus indicative for the target virus. Thereby, the presence or absence of the virus in the biological sample can be detected. The at least one target nucleic acid is thus a viral nucleic acid that may be selected from RNA and/or DNA. For the detection of a RNA virus, such as a coronavirus, the target nucleic acid is RNA. In this case, the RNA nucleic acid is preferably first transcribed in (C) to cDNA prior to performing the actual amplification reaction. As demonstrated in the examples, this can be done in a reverse transcription amplification reaction which is rapid and convenient for high throughput testing. For the detection of a DNA virus, the target nucleic is DNA and the amplification, such as a PCR, can be directly performed in (C). According to one embodiment, the target nucleic acid is provided by two or more viral target nucleic acids that are derived from the same virus to be detected. Also two or more different viruses may be detected in the same amplification reaction using the method according to the first aspect.

The virus to be detected may be a capsid or non-capsid virus. In a preferred embodiment, the virus is an enveloped virus. In one embodiment, the virus is a RNA virus, such as an enveloped RNA virus, and the amplification reaction is a reverse transcription amplification reaction. In preferred embodiments, the at least one target nucleic acid is a viral nucleic acid derived from a RNA virus. As is demonstrated in the examples, the technology of the invention is particularly suitable for providing a virus-inactivated biological sample for amplification based detection of viral target RNA derived from a RNA virus. The at least one target nucleic acid is in advantageous embodiments derived from a coronavirus,

The virus, the presence or absence of which in the biological sample may be detected using the technology of the present invention, may be a coronavirus, in particular a coronavirus infectious for humans. A human coronavirus as used herein in particular refers to a coronavirus that is infectious to a human (but e.g. may also infect other animals). According to a preferred embodiment, the at least one target nucleic acid is derived from a severe acute respiratory syndrome-related coronavirus, preferably severe acute respiratory syndrome coronavirus 2 (SARS-CoV-2) or severe acute respiratory syndrome coronavirus (SARS-CoV or SARS-CoV-1) or middle east respiratory syndrome (MERS). The at least one target nucleic acid that is amplified for the detection of the presence or absence of the virus is in particular embodiments a severe acute respiratory syndrome coronavirus 2 (SARS-CoV-2)-derived nucleic acid.

The coronavirus to be detected using the method according to the invention may in particular be a severe acute respiratory syndrome-related coronavirus, such as severe acute respiratory syndrome coronavirus 2 (SARS-CoV-2 also referred to as COVID-19) or severe acute respiratory syndrome (SARS-CoV or SARS-CoV-1). Accordingly, in a preferred embodiment, the target nucleic acid is derived from severe acute respiratory syndrome coronavirus 2 (SARS-CoV-2). A coronavirus may also be a middle east respiratory syndrome-related coronavirus, such as middle east respiratory syndrome coronavirus (MERS-CoV). In a further embodiment, a coronavirus is a human coronavirus 229E (HCoV-229E), HKU1 (HCoV-HKU1), NL63 (HCoV-NL63), OC43 (HCoV-OC43) or B814 (HCoV-B814), human enteric coronavirus (HECV). According to a further embodiment, the coronavirus is a betacoronavirus, sarbecovirus, murine hepatitis virus, murine coronavirus, hedgehog coronavirus, pipistrellus bat coronavirus, such as HKU5, HKU4, HKU1, HKU9, or HCOV-HKU1, tylonycteris derived coronavirus, rousettus derived coronavirus, Ty-BatCoV HKU5, or rhinolophus-derived coronavirus.

As is demonstrated in the examples, the technology of the invention is particularly suitable for testing biological samples for the presence of absence of SARS-CoV-2 and provides an advantageous, rapid and simple workflow that significantly improves existing SARS-CoV-2 testing methods as well as testing methods for other RNA (and DNA) viruses. According to one embodiment, the one or more target nucleic acids are thus derived from SARS-CoV-2, optionally wherein the target nucleic acid sequences are derived from the SARS-CoV-2 genes N, N1, N2, RdRP, E and Orf1b.

According to other embodiments, the virus to be detected is an influenza virus, such as influenza-A, influenza-B, influenza-C, influenza-D, influenza-H₁N1, or influenza H₃N2, a parainfluenza virus, a respiratory syncytial virus (RSV), an adenovirus, an enterovirus or a rhinovirus.

As disclosed herein, also two or more viruses can be detected using the method according to the invention. The method according to the invention may also be used for the detection of other pathogens apart from viruses and such embodiments are also covered by the disclosure and scope of the invention.

### Suitable and Preferred Embodiments of Step (C)

According to one embodiment, subjecting at least an aliquot or all (the desired amount) of the optionally pretreated virus-inactivated biological sample to an amplification reaction in (C) comprises contacting the optionally pretreated virus-inactivated biological sample with the components used for performing the amplification or reverse transcription amplification reaction thereby providing an amplification reaction admixture.

In one embodiment, the prepared amplification reaction admixture comprises:
(a) the (optionally pretreated) virus-inactivated biological sample;
(b) a DNA polymerase;
(c) optionally a reverse transcriptase, which is included in case a reverse transcription amplification is performed;
(d) an amplification reaction buffer comprising a Mg2+ source, a buffering agent and optionally further additives;
(e) nucleotides, preferably a dNTP mix, optionally wherein the nucleotides comprise modified nucleotides or dUTP; and
(f) primers for amplifying the one or more target nucleic acids and optionally probes.

According to one embodiment, the method comprises contacting the optionally pretreated virus-inactivated biological sample with an amplification master mix comprising components (b) to (e) and separately provided primers for amplifying the one or more target nucleic acids. This embodiment is particularly preferred for detecting RNA target nucleic acids, such as viral RNA. Further components that are commonly used in virus testing methods, such as probes (for quantitative RT-PCRs), internal controls etc. may also be included into the amplification reaction admixture. In embodiments, these further components are, however, also provided separately and are not included into the amplification master mix.

According to one embodiment, the method comprises contacting the optionally pretreated virus-inactivated biological sample with a direct amplification master mix comprising components (b) and (d) to (f) and optionally (c).

As discussed above, the (optionally pretreated) virus-inactivated biological sample may comprise the medium that was used for collecting the biological sample, such as a medium as disclosed herein. According to one embodiment, the ionic strength of amplification reaction buffer (d), the amplification master mix comprising components (b) to (e) and/or the direct amplification master mix comprising components (b) to (f) is reduced to thereby compensate the introduction of ions, in particular ions derived from alkali metal salts and/or chlorides, into the amplification reaction admixture due to the optionally pretreated virus-inactivated biological sample that contains the medium.

According to an advantageous embodiment, the ionic strength of the amplification reaction buffer (d) or the amplification master mix comprising components (b) to (e) is reduced to thereby compensate the introduction of ions, in particular ions derived from alkali metal salts and/or chlorides, into the amplification reaction admixture due to the optionally pretreated virus-inactivated biological sample that may contain the medium. Such a modified amplification buffer has been described in EP 20 214 412.7 and is incorporated herein by reference. This embodiment is advantageous to compensate the detrimental ions introduced by the medium thereby ensuring that the amplification can work properly enabling sensitive testing. Several suitable and preferred options to reduce the ionic strength and in particular the chloride concentration are described in the following and these options can also be used in combination and can be freely combined. In embodiments, the ionic strength of the amplification reaction buffer (d) or the amplification master mix comprising components (b) to (e) is reduced so that the salt concentration in the amplification reaction admixture that comprises the optionally pretreated virus-inactivated biological sample with all components used for performing the amplification or reverse transcription amplification reaction is ≤300 mM, preferably ≤250 mM or ≤220 mM. In case the (optionally pretreated) virus-inactivated biological sample comprises a high amount of salt that cannot be compensated by the described measures, a lower amount of the (optionally pretreated) virus-inactivated biological sample may be incorporated into the amplification reaction admixture to achieve a dilution effect. As disclosed herein, the disturbing ions such as chloride, sodium and/or ions derived from alkali metal salts usually originate from the biological sample, in particular if the biological sample is comprised in a medium as described above. Advantageously, the extraction composition that may be used to pretreat the virus-inactivated biological sample for direct amplification-based detection of the one or more target nucleic acids and which is thus contained in the optionally pretreated virus-inactivated biological sample may be free of anorganic salts, in particular chloride salts.

According to one embodiment, the amplification reaction buffer (d) has one or more, preferably two or more, more preferably three of more of the following characteristics:
(aa) the amplification reaction buffer (d) does not comprise sodium chloride in a concentration ≥30 mM ≥20 mM ≥15 mM, ≥10 mM or ≥5 mM and wherein preferably, the amplification reaction buffer (d) contains no sodium chloride;
(bb) the amplification reaction buffer (d) does not comprise potassium chloride in a concentration ≥30 mM ≥20 mM ≥15 mM, ≥10 mM or ≥5 mM and wherein preferably, the amplification reaction buffer (d) contains no potassium chloride;
(cc) the amplification reaction buffer (d) does not comprise potassium chloride or sodium chloride;
(dd) the alkali metal chloride concentration in the amplification reaction buffer (d) is ≤30 mM, ≤20 mM, ≤15 mM or ≤10 mM and wherein preferably, the amplification reaction buffer (d) does not contain alkali metal chlorides; and
(ee) the alkali metal salt concentration in the amplification reaction buffer (d) is ≤30 mM, ≤20 mM, ≤15 mM or ≤10 mM and wherein preferably, the amplification reaction buffer (d) does not contain alkali metal salts.

The amplification reaction buffer (d) does not comprise the enzymes (b) and (c). However, as described, components (b) to (e) may advantageously be provided in form of an amplification master mix. The use of such amplification master mix is common practice and convenient for the user.

In advantageous embodiments, the amplification reaction buffer (d) comprises a buffering agent that does not comprise chloride ions, optionally wherein the buffering agent is selected from the group consisting of tris(hydroxymethyl)aminomethane, N-(tri(hydroxymethyl)methyl)glycine, N,N-bis(2-hydroxyethyl)glycine, 3-(N-morpholino),propanesulphonic acid, N-(2-hydroxy¬ethyl)piperazine-N′-(2-ethanesulphonic acid), piperazine-1,4-bis(2-ethanesulphonic acid), N cyclohexyl-2-aminoethanesulphonic acid and 2-(N-morpholino)¬ethanesulphonic acid and preferably is selected from tris(hydroxymethyl)aminomethane and 3-(N-morpholino)-propanesulphonic acid.

In embodiments, the pH of the amplification reaction buffer (d) is adjusted with an acid that does not comprise chloride. This further reduces the chloride burden and therefore provides a robust method for processing different types of optionally pretreated virus-inactivated biological samples, in particular for performing a reverse transcription amplification. As disclosed herein, it allows the processing of optionally pretreated virus-inactivated biological samples that are comprised in salt-containing media or other media of high ionic strength that are pretreated using the extraction composition according to the present invention. As is demonstrated in the examples, the pH may be adjusted using an organic acid, preferably a carboxylic acid. In one embodiment, the pH of the amplification reaction buffer (d) is adjusted with a carboxylic acid selected from acetic acid, formic acid, propionic acid and butyric acid and preferably is adjusted with acetic acid. Other acids include mineral acids such as H₂SO₄ or HNO₃. The pH of the amplification reaction buffer (d) may be in the commonly used range, e.g. in the range of 6 to 10, 6.5 to 9.5, 7.0 to 9.5 and 7.5 to 9.0 such as about 8.0 to 8.5.

In further embodiments, the amplification reaction buffer (d) comprises a soluble magnesium salt as Mg²⁺ source that does not comprise chloride. This again allows to reduce the chloride concentration in the amplification reaction buffer (d). The same applies to an amplification master mix that comprises the amplification master mix (d). The soluble magnesium salt may be a magnesium salt of an organic acid. The magnesium salt may be selected from magnesium sulfate and magnesium acetate.

According to one embodiment, the amplification reaction buffer (d) is characterized in that:
(i) it does not comprise potassium chloride or sodium chloride;
(ii) it comprises a buffering agent that does not comprise chloride ions, optionally wherein the buffering agent is selected from tris(hydroxymethyl)aminomethane and 3-(N-morpholino)-propanesulphonic acid, and
(iii) the pH of the amplification reaction buffer (d) is adjusted with an organic acid, preferably a carboxylic acid.

According to one embodiment, the amplification master mix comprising components (b) to (e) or the direct amplification master mix comprising components (b) to (f) has one or more of the following characteristics:
(aa) it does not comprise sodium chloride in a concentration ≥50 mM, ≥20 mM, ≥15 mM or ≥10 mM and wherein preferably, it contains no sodium chloride;
(bb) it does not comprise potassium chloride in a concentration ≥100 mM, ≥75 mM, ≥60 mM or ≥50 mM, optionally wherein it contains no potassium chloride;
(cc) it does not comprise potassium chloride or sodium chloride;
(dd) the alkali metal chloride concentration in the amplification master mix or the direct amplification master mix is ≤100 mM, ≤75 mM, ≤60 mM, ≤50 mM or ≤45 mM, optionally wherein it does not contain alkali metal chlorides;
(ee) wherein the alkali metal salt concentration in the amplification master mix or the direct amplification master mix is ≤100 mM, ≤75 mM, ≤60 mM, ≤50 mM or ≤45 mM; and/or
(ff) the chloride ion concentration is ≤250 mM, ≤200 mM, ≤175 mM or ≤150 mM.

The amplification master mix comprising components (b) to (e) or the direct amplification master mix comprising components (b) to (f) has one or both of the following characteristics:
(aa) it comprises a buffering agent that does not comprise chloride ions, optionally wherein the buffering agent is selected from the group consisting of tris(hydroxymethyl)aminomethane, N-(tri(hydroxymethyl)methyl)glycine, N,N-bis(2-hydroxyethyl)glycine, 3-(N-morpholino)¬propanesulphonic acid, N-(2-hydroxy¬ethyl)piperazine-N′-(2-ethanesulphonic acid), piperazine-1,4-bis(2-ethanesulphonic acid), N cyclohexyl-2-aminoethanesulphonic acid and 2-(N-morpholino)ethanesulphonic acid and preferably is selected from tris(hydroxymethyl)aminomethane and 3-(N-morpholino)-propanesulphonic acid;
(bb) the pH is adjusted with an organic acid, preferably a carboxylic acid, optionally wherein the carboxylic acid selected from acetic acid, formic acid, propionic acid and butyric acid, preferably acetic acid.

Features (aa) and (bb) further reduce the ionic strength in the amplification master mix, thereby allowing to incorporate a higher amount of the optionally pretreated virus-inactivated biological sample into the amplification reaction admixture as the impact of additional ions e.g. deriving from chloride salts comprised in medium that contained the biological sample can be compensated thereby ensuring a robust performance of the amplification, such as a quantitative RT-PCR, even if crude biological samples comprising different types of transport media are processed.

According to one embodiment, the amplification master mix comprising components (b) to (e) or the direct amplification master mix comprising components (b) to (f) comprises a soluble magnesium salt as Mg²⁺ source that does not comprise chloride, optionally wherein the soluble magnesium salt is a magnesium salt of an organic acid, preferably selected from magnesium sulfate and magnesium acetate.

According to one embodiment, the amplification master mix comprising components (b) to (e) is characterized in that:
(i) it does not comprise sodium chloride in a concentration ≥50 mM, ≥20 mM, ≥15 mM or ≥10 mM and wherein preferably, it contains no sodium chloride;
(ii) it does not comprise potassium chloride in a concentration ≥100 mM, ≥75 mM, ≥60 mM or ≥50 mM, optionally wherein it contains no potassium chloride;
(iii) if chloride ions are present, the chloride ion concentration is ≤250 mM, ≤200 mM, ≤175 mM, ≤150 mM or ≤125 mM;
(iv) it comprises a buffering agent that does not comprise chloride ions, wherein preferably the buffering agent is selected from tris(hydroxymethyl)aminomethane and 3-(N-morpholino)-propanesulphonic acid;
(v) optionally wherein the pH is adjusted with an acid that does not comprise chloride, preferably an organic acid, more preferably a carboxylic acid, optionally wherein the carboxylic acid is acetic acid.

These embodiments that use accordingly optimized amplification reagents (e.g. amplification reaction buffer (d) and/or the amplification master mix wherein components (b) to (e) are provided in a single composition) allow to incorporate a high amount of optionally pretreated virus-inactivated biological sample into the amplification reaction admixture (e.g. 20% up to 40%, up to 50% or up to 60% of the total volume of the amplification reaction admixture that comprises all components used in the amplification, which preferably is a reverse transcription amplification) without detrimental inhibition of the amplification reaction by the components that are carried over from a salt-containing medium or other medium of high ionic strength into the (optionally pretreated) virus-inactivated biological sample and thus the amplification reaction. Alternatively, the amount of (optionally pretreated) virus-inactivated biological sample in the amplification reaction admixture can be reduced to ensure a high performance of the amplification reaction, in particular a reverse transcription amplification reaction.

According to one embodiment, the amplification buffer (d), the amplification master mix comprising components (b) to (e) or the direct amplification master mix comprising components (b) to (f) comprises one or more of the following additives:
an ammonium salt, optionally selected from ammonium sulfate and ammonium chloride;
polyethylene glycol;
N,N,N-trimethylglycine;
serum albumin;
a metal ion chelator, optionally EGTA;
glycerol;
fish gelatine;
PVP (polyvinylpyrrolidone);
DMSO; and
formamide.

These additives are commonly used in amplification reactions and the skilled person may choose appropriate concentrations following the general guidance provided in this application. Furthermore, the amplification buffer (d), the amplification master mix comprising components (b) to (e) or the direct amplification master mix comprising components (b) to (f) may comprise at least one substance that is capable of counteracting an inhibitory effect of a virus-deactivating substance that was used for virus-inactivation of the reverse transcription and/or amplification. Suitable embodiments for such counteracting substances for virus-deactivating substances are disclosed elsewhere herein and it is referred to the corresponding disclosure. Appropriate concentrations for such counteracting substances to ensure a good performance of the amplification/reverse transcription amplification can be determined by performing concentration series as illustrated in the examples.

As is also common in prior art methods, the prepared amplification reaction admixture comprising all components used in the amplification reaction, such as the reverse transcription amplification reaction (e.g. a quantitative RT-PCR) comprises:
(g) at least one internal control template and primers for amplifying said internal control template and optionally probes for detection.

As disclosed herein, the (optionally pretreated) virus-inactivated biological sample that is subjected to the amplification reaction may provide at least 20%, at least 30%, at least 40% or at least 45% of the total reaction volume of the amplification reaction, optionally wherein the optionally pretreated virus-inactivated biological sample that is subjected to the amplification reaction provides up to 60% or up to 50% of the total reaction volume of the amplification reaction, which preferably is a reverse transcription PCR or PCR. In preferred embodiments, the optionally pretreated virus-inactivated biological sample provides at least at least 30%, at least 40% or at least 45% of the total volume of the amplification reaction admixture which comprises the optionally pretreated virus-inactivated biological sample and all components necessary for performing the amplification. In embodiments, the optionally pretreated virus-inactivated biological sample provides up to 60% or up to 50% of the total volume of the amplification reaction admixture which comprises the optionally pretreated virus-inactivated biological sample and all components necessary for performing the amplification as is also demonstrated in the examples. The possibility to subject a high volume of the optionally pretreated virus-inactivated biological sample to the amplification reaction, such as the reverse transcription amplification reaction is advantageous because it increases the sensitivity. As disclosed herein and shown in the examples, despite processing a virus-inactivated biological sample without prior nucleic acid purification, the pretreatment step disclosed herein wherein the virus-inactivated biological sample is contacted with the extraction composition provides optionally pretreated virus-inactivated biological samples in which the target nucleic acids, including RNA target nucleic acids can be reliably identified with good sensitivity. Also the components of the extraction solution do not interfere with the subsequent amplification or reverse transcription amplification and can furthermore balance differences in the biological samples/counteract inhibitory effects of the at least one virus-deactivating substance thereby ensuring robust results.

Any kind of amplification can be performed, including but not limited to (i) reverse transcription amplification reaction, (ii) reverse transcription PCR, (iii) isothermal amplification reaction, (iv) polymerase chain reaction (PCR), (v) quantitative PCR, (vi) quantitative reverse transcription PCR, and (vii) digital PCR. Furthermore, the amplification may be a loop-mediated isothermal amplification (LAMP) or reverse transcription loop-mediated isothermal amplification (RT-LAMP). As used herein, the term “PCR” comprises PCR (polymerase chain reaction; DNA amplification) as well as RT-PCR (reverse transcription-polymerase chain reaction; RNA amplification). In particular preferred embodiments, the PCR is a semi-quantitative or more preferably a quantitative PCR, such as a quantitative reverse-transcription PCR. Performing a quantitative PCR is particularly preferred for pathogen testing. All components necessary for performing the chosen amplification type are included into the amplification reaction admixture that also includes the optionally pretreated virus-inactivated biological sample.

According to a preferred embodiment, the amplification reaction is a reverse-transcription amplification reaction, preferably a quantitative reverse-transcription polymerase chain reaction (RT-PCR). As is demonstrated in the examples, the method according to the present invention is particularly useful in order to amplify RNA target nucleic acids, which is a core application for virus testing, e.g. in order to detect the presence of absence of SARS-CoV-2 and other RNA viruses.

Advantageously, the steps of
contacting the virus-inactivated biological sample with the extraction composition,
incubating the admixture to provide the pretreated virus-inactivated biological sample, and
performing the amplification reaction, preferably a reverse-transcription amplification reaction,
   can be performed within the same reaction vessel. It is also within the scope of the present invention to perform the virus-inactivation in the same vessel in which the amplification reaction is performed. The reaction vessel may be provided by the well of a microtiter plate, such as a PCR plate.

A virus-deactivating substance may also be added to the amplification reaction comprising the biological sample to thereby provide a virus-inactivated composition and amplification reaction simultaneously. In this embodiment, providing in (A) and subjecting in (C) are performed concurrently and thus essentially in one step. E.g., after arrival at the lab, an aliquot of the biological sample is removed and transferred to a vessel that contains the virus-deactivating substance and at least some or all reagents/components necessary for amplification/reverse transcription amplification. The vessel may additionally comprise an extraction composition as described herein, or the individual components thereof. In embodiments, the reaction vessel comprises an extraction composition as disclosed herein which includes at least one virus-deactivating substance and at least some or all components necessary for amplification/reverse transcription amplification. The resulting admixture is incubated for efficient virus-inactivation (and optional pretreatment). The vessel may be sealed during incubation to avoid contaminations. The vessel may be provided by the well of a microtiter plate, such as a PCR plate, which is advantageous for high-throughput applications. The so obtained virus-inactivated and optionally pretreated biological sample that is present in contact with the reagents/components required for the amplification/reverse transcription amplification is then subjected to the actual amplification reaction. This can be done e.g. by transferring the vessel(s) comprising the virus-inactivated and optionally pretreated biological sample(s) to an instrument for performing the amplification reaction/reverse transcription amplification reaction. E.g. after virus-inactivation, the sealed PCR plates comprising the prepared virus-inactivated biological samples may be safely transferred to a thermocycler for performing the actual amplification reaction/reverse transcription amplification reaction.

### Further Embodiments of the Method According to the First Aspect

According to one embodiment, the biological sample is a respiratory biological sample and the virus is a RNA virus, such as a coronavirus, wherein the method comprises
(A) providing a virus-inactivated biological sample, wherein providing such sample comprises preparing a composition comprising the biological sample and at least one virus-deactivating substance, and wherein providing comprises
   contacting the biological sample with at least one virus-deactivating substance; wherein the virus-deactivating substance is selected from the group consisting of oxidizing agents, cationic surfactants, non-ionic surfactants, and zwitterionic surfactants; and
   incubating the composition to provide the virus-inactivated biological sample;
(B) optionally pretreating the biological sample, wherein optionally pretreating preferably comprises
   contacting an aliquot or all of the virus-inactivated biological sample with an extraction composition as defined above thereby providing an admixture; and
   incubating the admixture to provide the pretreated virus-inactivated biological sample;
      and
(C) subjecting at least an aliquot or all of the optionally pretreated virus-inactivated biological sample to a reverse transcription and amplification reaction, which preferably is a quantitative RT-PCR reaction, wherein the optionally pretreated virus-inactivated biological sample is in contact with the components used for performing the reverse transcription amplification reaction thereby providing an amplification reaction admixture, wherein the prepared amplification reaction admixture comprises
   (a) the optionally pretreated virus-inactivated biological sample;
   (b) a DNA polymerase;
   (c) a reverse transcriptase;
   (d) an amplification reaction buffer comprising a Mg²⁺ source, a buffering agent and optionally further additives;
   (e) nucleotides, preferably a dNTP mix; and
   (f) primers for reverse transcribing and amplifying the one or more target nucleic acids,
and performing the reverse transcription and amplification reaction to reverse transcribe and amplify at least one RNA target nucleic acid derived from the RNA virus.

According to one embodiment, the pretreatment step (B) is performed and wherein in (C) the pretreated virus-inactivated biological sample provides at least 30% or at least 40% of the total reaction volume of the prepared amplification reaction admixture; and wherein at least the steps of
contacting the virus-inactivated biological sample with the extraction composition to prepare the admixture,
incubating the admixture, and
performing the reverse-transcription amplification reaction,
   are performed within the same reaction vessel,
   and wherein the target nucleic acid is provided by one or more, preferably two or more, target nucleic acids derived from a severe acute respiratory syndrome-related coronavirus, preferably severe acute respiratory syndrome coronavirus 2 (SARS-CoV-2).

According to an advantageous embodiment, steps (B) and (C) are completed in 2 h or less, 1.5 h or less or 1 h or less.

Preferred embodiments of the methods according to the first aspect are again described in the following.
1. A method for detecting the presence or absence of a virus in a biological sample based on amplifying at least one target nucleic acid derived from the virus without prior nucleic acid purification, comprising
(A) providing a virus-inactivated biological sample, wherein providing such sample comprises preparing a composition comprising the biological sample and at least one virus-deactivating substance,
(B) optionally pretreating the biological sample,
(C) subjecting at least an aliquot or all of the optionally pretreated virus-inactivated biological sample to an amplification reaction and amplifying the at least one target nucleic acid, optionally wherein a reverse transcription reaction is performed in order to reverse transcribe RNA to cDNA prior to amplification.
2. The method according to item 1, wherein providing in (A) comprises
   contacting a biological sample with at least one virus-deactivating substance thereby preparing the composition, optionally wherein the biological sample is comprised in medium prior to contact with the at least one virus-deactivating substance; and
   incubating the composition.
3. The method according to item 1 or 2, wherein providing in (A) comprises
   preparing the composition by immersing a collected biological sample in medium, wherein the medium comprises at least one virus-deactivating substance; and
   incubating the composition, optionally wherein the medium is comprised in a container and wherein the container is closed by a closure prior to incubation.
4. The method according to item 1 or 2, wherein providing in (A) comprises
   immersing a biological sample in medium;
   contacting the biological sample comprised in medium with at least one virus-deactivating substance thereby preparing the composition; and
   incubating the composition.
5. The method according to any one of items 1 to 4, wherein the composition provided in (A) comprises medium that was used for collecting and/or storing the biological sample.
6. The method according to any one of items 1 to 5, wherein providing in (A) comprises heating the biological sample and/or the composition comprising the biological sample and the at least one virus-deactivating substance at a temperature to assist the virus inactivation, optionally wherein heating is performed at a temperature of ≥50° C., ≥55° C. or ≥60, preferably ≥75° C., ≥80° C. or ≥85° C., more preferably ≥90° C. or 95° C.
7. The method according to any one of items 1 to 6, wherein the composition provided in (A) comprises two or more virus-deactivating substances.
8. The method according to any one of items 1 to 7, wherein an inhibitory effect of the at least one virus-deactivating substance on the amplification reaction and/or the reverse transcription is counteracted prior to or during performing the amplification reaction in (C).
9. The method according to item 8, wherein an inhibitory effect of the at least one virus-deactivating substance is counteracted by addition of at least one substance that can counteract the inhibitory effect of the at least one virus-deactivating substance, wherein preferably the activity of the DNA polymerase, and the reverse transcriptase if a reverse transcription is performed, is restored.
10. The method according to item 9, wherein the at least one substance that can counteract an inhibitory effect of the at least one virus-deactivating substance is added in pretreatment step (B) and/or is included in the amplification reaction of (C).
11. The method according to any one of items 1 to 10, wherein in (A) the composition comprising the biological sample and the at least one virus-deactivating substance comprises the at least one virus-deactivating substance in a concentration wherein it supports or achieves virus-inactivation.
12. The method according to any one of items 1 to 11, wherein the virus-inactivated biological sample provided in (A) comprises the at least one virus-deactivating substance in a concentration where it does not interfere with the amplification reaction when at least an aliquot or all of the optionally pretreated virus-inactivated biological sample is subjected to an amplification reaction in (C).
13. The method according to any one of items 1 to 12, wherein the virus-inactivated biological sample provided in (A) comprises the at least one virus-deactivating substance in a concentration where it does not interfere with the reverse transcription reaction and the amplification reaction when at least an aliquot or all of the optionally pretreated virus-inactivated biological sample is subjected to such amplification reaction in (C).
14. The method according to any one of items 1 to 13, wherein the optionally pretreated virus-inactivated biological sample does not interfere with the amplification reaction when at least an aliquot or all of the optionally pretreated virus-inactivated biological sample is subjected to an amplification reaction in (C).
15. The method according to any one of items 1 to 14, wherein the optionally pretreated virus-inactivated biological sample does not interfere with the reverse transcription reaction and the amplification reaction when at least an aliquot or all of the optionally pretreated virus-inactivated biological sample is subjected to an amplification reaction in (C).
16. The method according to any one of items 1 to 15, wherein at least one virus-deactivating substance is used which is a disinfectant or a surfactant.
17. The method according to any one of items 1 to 16, wherein the virus-deactivating substance is an oxidizing agent, optionally selected from the group consisting of
(aa) iodine-releasing agents;
(bb) peroxide-based disinfectants, such as hydrogen peroxide and peroxyacetic acid;
(cc) chlorine-releasing disinfectants, such as sodium hypochlorite.
18. The method according to item 16 or 17, wherein a iodophore is used as virus-deactivating substance, preferably povidone iodine.
19. The method according to any one of items 16 to 18,
   wherein the composition comprising the biological sample and the virus-deactivating substance comprises a disinfectant as virus-deactivating substance, wherein the concentration of the disinfectant lies in the range of 0.001 to 5%, optionally 0.01% to 1% or 0.02% to 0.5%; and/or
   wherein the virus-deactivating substance is a disinfectant and wherein the concentration of the disinfectant in the amplification reaction provided in (C) lies in the range of 0.0005% to 1%, optionally 0.0035% to 0.35% or 0.007% to 0.07%, optionally wherein the disinfectant is an oxidizing agent, preferably a iodophore such as povidone iodine.
20. The method according to any one of items 16 to 19, wherein an inhibitory effect of the disinfectant on the amplification reaction and/or the reverse transcription is counteracted prior to or during performing the amplification reaction in (C) by addition of at least one substance that can counteract the inhibitory effect of the disinfectant, wherein preferably the virus-deactivating disinfectant is an oxidizing agent and the counteracting substance is a reducing agent.
21. The method according to item 20, wherein the at least one substance that can counteract the inhibitory effect of the disinfectant is a reducing agent and wherein the reducing agent is added in pretreatment step (B) and/or is included in the amplification reaction of (C), wherein preferably, the disinfectant is povidone iodine.
22. The method according to any one of items 1 to 21, wherein at least one virus-deactivating substance is used which is a surfactant selected from the group consisting of (i) cationic surfactants, (ii) non-ionic surfactants, (iii) anionic surfactants, and (iv) zwitterionic surfactants.
23. The method according to item 22, wherein the virus-deactivating substance is a cationic surfactant which is a quaternary ammonium compound, preferably a quaternary ammonium salt, optionally wherein a mixture of quaternary ammonium compounds is used as cationic surfactant.
24. The method according to item 23, wherein the quaternary ammonium compound comprises four organic substituents on the nitrogen atom, wherein the substituents are independently selected from alkyl, aryl and heterocycles, optionally wherein the alkyl chain length is selected from C₁ to C₂₀.
25. The method according to item 23 or 24, wherein the cationic surfactant used as virus-deactivating substance has at least one of the following characteristics:
(aa) it is a tetraalkylammonium salt, wherein preferably, (i) at least one alkyl substituent has a chain length selected from C₈ to C₂₀ or C₁₀ to C₁₈ and (ii) two or three alkyl substituents have a chain length selected from C₁ to C₆ or C₁ to C₄, preferably C₁ or C₂;
(bb) it is a dialkyl dimethyl ammonium salt, wherein the chain length of the alkyl groups is selected from C₈ to C₁₆ or C₁₀ to C₁₂, optionally wherein the chain length of the alkyl groups is the same;
(cc) it is an alkyltrimethylammonium salt, wherein preferably, the chain length of the alkyl group is selected from C₈ to C₂₀ or C₁₀ to C₁₈;
(dd) it is an alkyl/aryl-quaternary ammonium salt, such as an alkylbenzyldimethylammonium salt, wherein preferably the chain length of the alkyl group is selected from C₈ to C₂₀ or C₈ to C₁₈;
(ee) the anion is selected from a halide and a sulfate, preferably a halide selected from chloride and bromide.
26. The method according to item 23 or 24, wherein the cationic surfactant used as virus-deactivating substance is selected from didecyldimethylammonium chloride, benzalkonium chloride and an alkyltrimethylammonium salt.
27. The method according to any one of items 22 to 26,
   wherein the composition comprising the biological sample and the virus-deactivating substance comprises a cationic surfactant as virus-deactivating substance, wherein the concentration of the cationic surfactant lies in the range of 0.0001% to 3%, optionally 0.0005% to 1%, 0.001% to 0.5%, 0.005% to 0.1% or 0.01% to 0.05%; and/or
   wherein the virus-deactivating substance is a cationic surfactant and wherein the concentration of the cationic surfactant in the amplification reaction provided in (C) lies in the range of 0.0001% to 0.1%, optionally 0.0002% to 0.05%, 0.00025% to 0.025% or 0.0025% to 0.01%.
28. The method according to any one of items 22 to 27, wherein an inhibitory effect of the cationic surfactant on the amplification reaction and/or the reverse transcription is counteracted prior to or during performing the amplification reaction in (C) by addition of at least one substance that can counteract the inhibitory effect of the cationic surfactant.
29. The method according to item 28, wherein the at least one substance that can counteract the inhibitory effect of the cationic surfactant is a surfactant and wherein the surfactant is added in pretreatment step (B) and/or is included in the amplification reaction of (C).
30. The method according to item 29, wherein the surfactant that counteracts the inhibitory effect of the cationic surfactant is an anionic surfactant, optionally SDS, optionally wherein the cationic surfactant used as virus-deactivating substance is a quaternary ammonium salt, preferably didecyldimethylammonium chloride.
31. The method according to item 22, wherein the virus-deactivating substance is a non-ionic surfactant which is an alcohol ethoxylate or an alkyl glycoside, optionally wherein a mixture of different alcohol ethoxylates, a mixtures of different alkyl glycosides or a mixtures of one or more alcohol ethoxylates and one or more alkyl glycosides is used.
32. The method according to item 31, wherein the alcohol ethoxylate is selected from the group consisting of seed oil alcohol alkoxylates and 2-ethyl hexanol ethoxylated propoxylated copolymers.
33. The method according to item 32, wherein the alcohol ethoxylate comprises three ((EO)₃), four ((EO)₄), six ((EO)₆), seven ((EO)₇), or nine ((EO)₉) ethoxylated moieties.
34. The method according to item 32 or 33, wherein the seed oil alcohol alkoxylate is of the formula X(C₃H₆O)ₘ(C₂H₄O)ₙ, wherein X is an aromatic or aliphatic C₆ to C₁₂ alkyl group, which is linear, branched or cyclic and optionally further substituted, m is 3 or 4 and n is an integer from 4 to 9.
35. The method according to item 34, wherein the seed oil alcohol alkoxylate used as virus-deactivating non-ionic surfactant is of the formula X(C₃H₆O)ₘ(C₂H₄O)ₙ, wherein X is an aromatic or aliphatic C₆ to C₁₂ alkyl group, which is linear, branched or cyclic and optionally further substituted, m is 3 or 4 and n is 9.
36. The method according to item 34, wherein the seed oil alcohol alkoxylate used as virus-deactivating non-ionic surfactant is of the formula X(C₃H₆O)ₘ(C₂H₄O)ₙ, wherein X is an aromatic or aliphatic C₆ to C₁₂ alkyl group, which is linear, branched or cyclic and optionally further substituted, m is 3 or 4 and n is 7.
37. The method according to item 34, wherein the seed oil alcohol alkoxylate used as virus-deactivating non-ionic surfactant is of the formula X(C₃H₆O)ₘ(C₂H₄O)ₙ, wherein X is an aromatic or aliphatic C₆ to C₁₂ alkyl group, which is linear, branched or cyclic and optionally further substituted, m is 3 or 4 and n is 4.
38. The method according to item 32 or 33, wherein the 2-ethyl hexanol ethoxylated propoxylated copolymer used as virus-deactivating non-ionic surfactant is of the formula C₈H₁₈O(C₃H₆O)ₘ(C₂H₄O)ₙ, wherein m is an integer from 1 to 12 and n is an integer from 3 to 9.
38. The method according to item 32 or 33, wherein the 2-ethyl hexanol ethoxylated propoxylated copolymer used as virus-deactivating non-ionic surfactant is of the formula C₈H₁₈O(C₃H₆O)ₘ(C₂H₄O)ₙ, wherein m is an integer from 1 to 12 and n is an integer from 3 to 9.
39. The method according to item 32 or 33, wherein the 2-ethyl hexanol ethoxylated propoxylated copolymer is of the formula C₈H₁₈O(C₃H₆O)ₘ(C₂H₄O)ₙ, wherein m is an integer from 1 to 12, preferably an integer from 1 to 8 and n is 9.
40. The method according to item 32 or 33, wherein the 2-ethyl hexanol ethoxylated propoxylated copolymer is of the formula C₈H₁₈O(C₃H₆O)ₘ(C₂H₄O)ₙ, wherein m is an integer from 1 to 12, preferably an integer from 1 to 8 and n is 6.
41. The method according to item 32 or 33, wherein the 2-ethyl hexanol ethoxylated propoxylated copolymer is of the formula C₈H₁₈O(C₃H₆O)ₘ(C₂H₄O)ₙ, wherein m is an integer from 1 to 12, preferably an integer from 1 to 8 and n is 3.
42. The method according to item 22, wherein the virus-deactivating substance is a polyoxyethylene-based non-ionic surfactant, preferably selected from the group consisting of
(aa) polyoxyethylene fatty acid esters, in particular polyoxyethylene sorbitan fatty acid esters; and
(bb) polyoxyethylene alkylphenyl ether, optionally wherein the polyoxyethylene alkyl phenyl ether is selected from the group consisting of polyoxyethylene nonylphenyl ether and polyoxyethylene isooctylphenyl ether.
43. The method according to item 42, wherein the non-ionic surfactant is a polyoxyethylene fatty acid ester, comprising
   a fatty acid derived from laureate, palmitate, stearate and oleate,
   a polyoxyethylene component containing from 2 to 150, 4 to 100, 6 to 50 or 6 to 30 (CH₂CH₂O) units,
wherein preferably, the polyoxyethylene fatty acid ester is selected from polysorbate 20, polysorbate 40, polysorbate 60 and polysorbate 80,
optionally wherein said non-ionic surfactant is used in combination with at least one additional virus-deactivating substance and/or heating in order to provide a virus-inactivated biological sample in (A).
44. The method according to item 42, wherein the non-ionic surfactant is a polyoxyethylene alkyl phenyl ether, comprising an alkyl group having from 5 to 15 carbon atoms, wherein the polyoxyethylene alkyl phenyl ether is preferably selected from the group consisting of polyoxyethylene nonylphenyl ether and polyoxyethylene isooctylphenyl ether.
45. The method according to item 22, wherein the virus-deactivating substance is a linear or branched alkyl polyethylene glycol ether, preferably of the general formula C₁₃H₂₁O(CH₂CH₂O)_{y}H, wherein y is an integer in the range of 5 to 14.
46. The method according to item 45, wherein the virus-deactivating substance is a C₁₀-Guerbet-based alkyl polyethylene glycol ether, preferably selected from the general formula C₁₀H₂₁(CH₂CH₂O)ₓH, wherein x is an integer in the range of 3 to 10 or 14.
47. The method according to item 46, wherein the virus-deactivating substance is a C₁₀-Guerbet-based alkyl polyethylene glycol ether is of the formula C₁₀H₂₁(CH₂CH₂O)₉H and has a HBL value in the range of 14 to 15, preferably of 14.5.
48. The method according to item 22, wherein the virus-deactivating substance is an alkyl glycoside-based non-ionic surfactant, optionally selected from the group consisting of decyl polyglucoside, decyl β-D glucopyranoside, lauryl glucoside, n-octyl β-D glucopyranoside.
49. The method according to any one of the preceding items, wherein the composition comprising the biological sample and the virus-deactivating substance comprises a non-ionic surfactant as virus-deactivating substance, wherein the concentration of the non-ionic surfactant lies in the range of 0.1% to 25%, optionally 0.2% to 2%.
50. The method according to any one of the preceding items, wherein the virus-deactivating substance is a non-ionic surfactant and wherein the concentration of the non-ionic surfactant in the amplification reaction provided in (C) lies in the range of 0.01% to 10%, 0.035% to 1.5%, optionally 0.05% to 1%.
51. The method according to item 22, wherein the virus-deactivating substance is an anionic surfactant, optionally selected from sodium dodecyl sulfate, N-lauroyl sarcosine and caprylic acid.
52. The method according to item 51, wherein an inhibitory effect of the anionic surfactant on the amplification reaction and/or the reverse transcription is counteracted prior to or during performing the amplification reaction in (C) by addition of at least one substance that can counteract the inhibitory effect of the anionic surfactant.
53. The method according to item 52, wherein the at least one substance that can counteract the inhibitory effect of the anionic surfactant is a surfactant and wherein the surfactant is added in pretreatment step (B) and/or is included in the amplification reaction of (C).
54. The method according to item 53, wherein the surfactant that counteracts the inhibitory effect of the anionic surfactant is a non-ionic surfactant or a cationic surfactant.
55. The method according to item 54, wherein the anionic surfactant used as virus-deactivating substance is SDS and the surfactant that counteracts the inhibitory effect of SDS is a quaternary ammonium salt, such as an alkyl- or aryl- or alkyl/aryl-quaternary ammonium salt, preferably didecyldimethylammonium chloride.
56. The method according to 22, wherein the virus-deactivating substance is an amine oxide-based zwitterionic surfactant.
57. The method according to item 56, wherein the amine oxide-based zwitterionic surfactant comprises a C8 to C18 alkyl group, optionally a C10 to C16 alkyl group or a C12 to C14 alkyl group, such as a C12 alkyl group.
58. The method according to item 56 or 57, wherein the amine oxide-based zwitterionic surfactant is lauryldimethylamine oxide.
59. The method according to any one of items 56 to 58, wherein the composition comprising the biological sample and the virus-deactivating substance comprises a zwitterionic surfactant as virus-deactivating substance, wherein the concentration of the zwitterionic surfactant lies in the range of 0.001% to 5.0%, optionally 0.02% to 1.5% (v/v) or 0.05% to 1% (v/v).
60. The method according to any one of items 56 to 59, wherein the virus-deactivating substance is a zwitterionic surfactant and wherein the concentration of the zwitterionic surfactant in the amplification reaction provided in (C) lies in the range of 0.002% to 1%, optionally 0.007% to 0.7%, optionally 0.01% to 0.1%.
61. The method according to any one of items 1 to 60, the virus-deactivating substance is an organophosphorous compound, optionally tri-n-butyl phosphate (TBP).
62. The method according to any one of items 1 to 61, wherein at least one virus-deactivating substances is used that is selected from the group consisting of quaternary ammonium salts, seed oil alcohol alkoxylates, 2-ethyl hexanol ethoxylated propoxylated copolymers, polyoxyethylene alkyl phenyl ether, SDS and an amine oxide-based zwitterionic surfactant comprising a C10 to C16 alkyl group.
63. The method according to any one of items 1 to 62, wherein at least one virus-deactivating substances is used that is selected from the group consisting iodine-releasing agents such as iodophores, quaternary ammonium salts and non-ionic surfactants.
64. The method according to any one of items 1 to 65, having one or more of the following characteristics:
(aa) the at least one target nucleic acid is a viral nucleic acid selected from RNA and/or DNA;
(bb) the target nucleic acid is provided by two or more viral target nucleic acids derived from the same virus to be detected;
(cc) the one or more target nucleic acids are RNA targets and wherein the amplification reaction is a reverse transcription amplification reaction.
65. The method according to any one of items 1 to 64, wherein the virus is an enveloped virus.
66. The method according to any one of items 1 to 65, wherein the virus is an RNA virus, optionally an enveloped RNA virus, and wherein the amplification reaction is a reverse transcription amplification reaction.
67. The method according to any one of items 1 to 66, wherein virus is a coronavirus and the at least one target nucleic acid to be amplified is derived from a coronavirus, in particular a coronavirus infectious for humans.
68. The method according to any one of items 1 to 67, wherein the at least one target nucleic acid to be amplified is derived from a severe acute respiratory syndrome-related coronavirus, preferably severe acute respiratory syndrome coronavirus 2 (SARS-CoV-2), severe acute respiratory syndrome coronavirus (SARS-CoV or SARS-CoV-1) or Middle East Respiratory Syndrome (MERS), optionally wherein the one or more target nucleic acids are derived from SARS-CoV-2, optionally wherein the one or more target nucleic acid sequences are selected from the SARS-CoV-2 genes N, N1, N2, RdRP, E and Orf1b.
69. The method according to any one of items 1 to 68, wherein the biological sample has one or more of the following characteristics:
(aa) it is a bodily sample;
(bb) it is a human sample;
(cc) it is a respiratory specimen, optionally collected from the upper or lower respiratory tract;
(dd) it is an oral sample, a nasal sample, a nasopharyngeal sample, an oropharyngeal sample, or a throat sample;
(ee) it is selected from the group consisting of saliva, sputum, spittle, mucus, drool, bronchoalveolar lavage, pharynx secretions, nasal secretions, nasopharyngeal secretions, salivary secretions, a swab or smear sample derived from mouth, nose and/or throat and a combination of the foregoing.
70. The method according to item 69, wherein the biological sample is selected from nasopharyngeal, oropharyngeal and nasal samples, preferably selected from a nasopharyngeal, oropharyngeal or nasal swab, smear or wash/aspirate samples, more preferably selected from swab or smear samples.
71. The method according to item 69, wherein the biological sample is selected from saliva, sputum and mucus.
72. The method according to any one of items 1 to 71, wherein the biological sample is comprised in medium and wherein the medium has one or more of the following characteristics:
(aa) the medium is a transport medium, optionally a transport medium for swab and/or smear samples;
(bb) the medium is an aqueous solution;
(cc) the medium is a saline solution suitable to keep the osmotic pressure in cells comprised in the biological sample when the medium is in contact with the biological sample;
(dd) the medium stabilizes the at least one target nucleic acid against degradation; and/or
(ee) the medium stabilizes cells and/or viral particles comprised in the biological sample.
73. The method according to item 72, wherein the medium has at least one of the following characteristics:
(aa) it comprises Hank's balanced salt solution;
(bb) it is a salt containing solution;
(cc) it is a physiological salt solution;
(dd) it is a solution comprising 0.7% to 1.2% (w/v) or 0.8% to 1% (w/v) alkali metal salts;
(ee) it is a 0.9% (w/v) sodium chloride solution;
(ff) it is a phosphate buffer, optionally a PBS buffer;
(gg) the medium comprises or consists of Hank's balanced salt solution, Universal Transport Medium (UTM), Viral Transport Medium (VTM) or a medium having a total salt concentration in a range+/−30% or +/−20% compared to one or more of the aforementioned media.
74. The method according to item 73, wherein the medium comprising the biological sample is a salt containing solution and wherein the total salt concentration in the medium comprising the biological sample lies in a range of 50 mM to 250 mM, such as 75 mM to 225 mM, 100 mM to 200 mM, 120 mM to 175 mM or 125 mM to 150 mM.
75. The method according to item 73 or 74, wherein the medium comprises at least one virus-deactivating substance.
76. The method according to item 75, wherein the composition that is formed when contacting the medium comprising at least one virus-deactivating substance with the biological sample is a composition as defined in any one of the preceding items.
77. The method according to any one of items 1 to 76, wherein pretreating in (B) is performed and wherein pretreating comprises
   contacting the biological sample with an extraction composition, preferably an extraction solution, comprising
   (a) at least one surfactant,
   (b) at least one nuclease inhibitor, and/or
   (c) at least one reducing agent, thereby providing an admixture;
   and
   incubating the admixture to provide a pretreated virus-inactivated biological sample; optionally wherein the pretreated virus-inactivated biological sample that is subjected to the amplification reaction provides at least 20%, at least 30% or at least 40% of the total reaction volume of the amplification reaction, optionally wherein the amount of the pretreated biological sample that is subjected to the amplification reaction provides up to 50% or up to 60% of the total reaction volume of the amplification reaction.
78. The method according to item 77,
   wherein per variant A, pretreatment in (B) is performed after providing the virus-inactivated biological sample in (A); or
   wherein per variant B, virus-inactivation (A) and pretreatment (B) are performed concurrently, optionally by contacting the biological sample with an extraction composition comprising the virus-deactivating substance and (a) at least one surfactant, (b) at least one nuclease inhibitor, and/or (c) at least one reducing agent and incubating the provided admixture to provide a pretreated virus-inactivated biological sample.
79. The method according to item 77 or 78, wherein the surfactant comprised in the extraction composition is selected from non-ionic and zwitterionic surfactants and wherein preferably, the surfactant is a non-ionic surfactant.
80. The method according to item 79, wherein the non-ionic surfactant is a polyoxyethylene-based non-ionic surfactant, preferably selected from the group consisting of
(aa) polyoxyethylene fatty acid esters, in particular polyoxyethylene sorbitan fatty acid esters;
(bb) polyoxyethylene fatty alcohol ether;
(cc) polyoxyethylene alkylphenyl ether, optionally wherein the polyoxyethylene alkyl phenyl ether is selected from the group consisting of polyoxyethylene nonylphenyl ether and polyoxyethylene isooctylphenyl ether; and
(dd) polyoxyethylene-polyoxypropylene block copolymers.
81. The method according to item 80, wherein the extraction composition comprises a polyoxyethylene fatty acid ester, comprising
   a fatty acid derived from laureate, palmitate, stearate and oleate,
   a polyoxyethylene component containing from 2 to 150, 4 to 100, 6 to 50 or 6 to 30 (CH₂CH₂O) units,
wherein preferably, the polyoxyethylene fatty acid ester is selected from polysorbate 20, polysorbate 40, polysorbate 60 and polysorbate 80.
82. The method according to item 80, wherein the extraction composition comprises a polyoxyethylene fatty alcohol ether, comprising
   a fatty alcohol component having from 6 to 22 carbon atoms, and
   a polyoxyethylene component containing from 2 to 150, 4 to 100, 6 to 50 or 6 to 30 (CH₂CH₂O) units,
wherein the polyoxyethylene fatty alcohol ether is preferably selected from the group consisting of polyoxyethylene lauryl ether, polyoxyethylene cetyl ether, polyoxyethylene stearyl ether and polyoxyethylene oleyl ether.
83. The method according to item 79, wherein the surfactant is a zwitterionic surfactant, optionally a betaine such as N,N,N trimethylglycine.
84. The method according to any one of item 77 to 83, having one or more of the following characteristics:
(i) the virus-deactivating substance differs from the surfactant comprised in the extraction composition;
(ii) the virus-inactivated biological sample is contacted with an extraction composition comprising a surfactant that is suitable to counteract an inhibitory effect of the at least one virus-deactivating substance comprised in the virus-inactivated biological sample on the amplification reaction and/or the reverse transcription reaction performed in (C).
85. The method according to any one of items 77 to 84, having one or more of the following characteristics:
(aa) wherein the extraction solution comprises the surfactant in a concentration that lies in a range of 0.1% to 30% (w/v), optionally selected from the ranges of 0.5% to 25% (w/v), 0.7% to 20% (w/v), 1% to 15% (w/v), 1.2% to 10% (w/v), 1.5% to 8% (w/v) and 2% to 5% (w/v); and/or
(bb) wherein the admixture comprising the virus-inactivated biological sample in contact with the extraction composition comprises the surfactant originating from the extraction composition in a concentration that lies in a range of 0.075% to 20% (w/v), optionally selected from the ranges of 0.1% to 15% (w/v), 0.15% to 15% (w/v), 0.2% to 10% (w/v), 0.25% to 8% (w/v), 0.3% to 5% (w/v), 0.35% to 3% (w/v) and 0.4% to 2% (w/v).
86. The method according to one or more of items 77 to 85, wherein the nuclease inhibitor comprised in the extraction composition is an RNase inhibitor or a DNase inhibitor, optionally wherein the extraction composition comprises two or more nuclease inhibitors, such as (i) two or more RNase inhibitors, (ii) two or more DNase inhibitors or (iii) one or more RNase inhibitors and one or more DNase inhibitors.
87. The method according to item 86, wherein a reverse transcription reaction and/or an amplification reaction can be performed in the presence of the comprised nuclease inhibitor.
88. The method according to item 86 or 87, wherein the nuclease inhibitor is an RNAase inhibitor, wherein preferably, the RNase inhibitor is a proteinaceous RNase inhibitor such as RNasin.
89. The method according to any one of items 77 to 88, wherein the extraction composition comprises the reducing agent and wherein the reducing agent is capable of destroying disulfide bonds and denaturing proteins, optionally wherein the reducing agent comprised in the extraction composition assists in liquefying the biological sample.
90. The method according to any one of items 77 to 88, wherein the extraction composition comprises the reducing agent and wherein the comprised reducing agent counteracts an inhibitory effect of the at least one virus-deactivating substance comprised in the virus-inactivated biological sample on the amplification reaction and/or the reverse transcription reaction performed in (C).
91. The method according to any one of items 77 to 90, wherein the reducing agent is selected from Tris(carboxyethyl)phosphine (TCEP), Dithiothreitol (DTT), N-acetyl cysteine, THPP (Tris(hydroxypropyl)phosphine), 1-thioglycerol and beta-mercaptoethanol, optionally wherein the extraction composition comprises Tris(carboxyethyl)phosphine (TCEP).
92. The method according to any one of items 77 to 91, having one or more of the following characteristics:
(aa) wherein the extraction composition comprises the reducing agent in a concentration that lies in a range of 0.3 mM to 50 mM, optionally selected from the ranges of 0.5 mM to 25 mM, 1 mM to 20 mM, 1.5 mM to 15 mM and 2 mM to 10 mM or 2 mM to 5 mM; and/or
(bb) wherein the admixture comprising the virus-inactivated biological sample in contact with the extraction composition comprises the reducing agent in a concentration that lies in a range of 0.1 mM to 15 mM, optionally selected from the ranges of 0.2 mM to 10 mM, 0.25 mM to 8 mM, 0.3 mM to 5 mM, 0.35 mM to 2 mM and 0.4 mM to 1.5 mM.
93. The method according to any one of items 77 to 92, wherein the extraction composition comprise a reducing agent selected from Tris(carboxyethyl)phosphine (TCEP), Dithiothreitol
(DTT), N-acetyl cysteine, THPP (Tris(hydroxypropyl)phosphine) and 1-thioglycerol in a concentration that lies in the range of 1 mM to 10 mM or 2 mM to 5 mM.
94. The method according to any one of items 77 to 93, wherein the extraction composition, which preferably is a liquid composition, is selected from the following embodiments:
(i) the extraction composition comprises
(a) at least one non-ionic surfactant,
(b) at least one proteinaceous RNase inhibitor, and
(c) at least one reducing agent;
(ii) the extraction composition comprises
(a) at least one polyoxyethylene-based non-ionic surfactant,
(b) at least one proteinaceous RNase inhibitor, and
(c) at least one reducing agent selected from Tris(carboxyethyl)phosphine (TCEP), Dithiothreitol (DTT), N-acetyl cysteine, THPP (Tris(hydroxypropyl)phosphine) and 1-thioglycerol;
(iii) the active ingredients of the extraction composition essentially consists of
(a) a non-ionic surfactant, preferably a polyoxyethylene-based non-ionic surfactant,
(b) a proteinaceous RNase inhibitor, and
(c) a reducing agent, preferably selected from Tris(carboxyethyl)phosphine (TCEP), Dithiothreitol (DTT), N-acetyl cysteine, THPP (Tris(hydroxypropyl)phosphine) and 1-thioglycerol;
(iv) the extraction composition comprises
(a) at least one polysorbate,
(b) at least one proteinaceous RNase inhibitor, and
(c) Tris(carboxyethyl)phosphine (TCEP);
(v) the active ingredients of the extraction composition essentially consists of
(a) at least one polysorbate,
(b) at least one proteinaceous RNase inhibitor, and
(c) Tris(carboxyethyl)phosphine (TCEP).
95. The method according to any one of items 77 to 94, wherein the extraction composition has a pH in the range of 6.0 to 9.0, optionally 6.0 to 8.5, 6.3 to 8.0 or 6.5 to 7.5.
96. The method according to any one of items 77 to 95, wherein the extraction composition used in (B) does not comprise one or more, two or more, three or more or all of the following components:
   an ionic surfactant;
   a chaotropic salt;
   chloride ions in a concentration exceeding 10 mM, wherein preferably the extraction composition does not comprise chloride ions;
   an aliphatic C1-C5 alcohol; and/or
   a proteinase enzyme.
97. The method according to any one of items 77 to 96, having one or more of the following characteristics:
(aa) the admixture provided in (B) is incubated for 1 to 60 min, 1 to 30 min, 1 to 20 min, 1 to 15 min, 1 to 10 min, 1.5 to 5 min or 2 to 3 min; and/or
(bb) preparing the admixture in (B) comprises agitating the virus-inactivated biological sample in contact with the extraction composition, optionally wherein the admixture is aspirated and dispensed and/or vortexed for agitation;
(cc) the steps of contacting the virus-inactivated biological sample with the extraction composition and incubating the admixture in (B) are carried out at ambient temperature and/or ice, optionally wherein all steps of the method apart from the enzymatic reaction are carried out at ambient temperature and/or ice.
98. The method according to any one of items 1 to 97, wherein the method is characterized by one or more of the following features:
(aa) wherein if pretreatment step (B) is performed, it does not involve heating the biological sample in contact with the extraction composition to a temperature 75° C., 70° C., 65° C., 60° C., 55° C., 50° C., 45° C. or 40° C. for at least 2 min prior to subjecting at least an aliquot or all of the pretreated virus-inactivated biological sample to the amplification reaction;
(bb) wherein if pretreatment step (B) is performed, it does not involve heating the biological sample in contact with the extraction composition to a temperature that would denature a comprised proteinaceous RNase inhibitor prior to subjecting at least an aliquot or all of the pretreated virus-inactivated biological sample to the amplification reaction;
(cc) it does not involve centrifuging the optionally pretreated virus-inactivated biological sample prior to subjecting at least an aliquot or all of the optionally pretreated virus-inactivated biological sample to the amplification reaction;
(dd) it does not involve removing cellular components from the optionally pretreated virus-inactivated biological sample prior to subjecting at least an aliquot or all of the optionally pretreated virus-inactivated biological sample to the amplification reaction;
(ee) it does not comprise purifying the target nucleic acid prior to subjecting at least an aliquot or all of the optionally pretreated virus-inactivated biological sample to the amplification reaction, and/or
(ff) pretreatment step (B) is performed in the presence of some or all components necessary for performing the amplification in (C).
99. The method according to any one of items 77 to 98, wherein providing the virus-inactivated biological sample in (A) comprises
(aa) heating the biological sample prior to contacting with a virus-deactivating substance, or
(bb) heating the composition comprising the biological sample and the virus-deactivating substance,
wherein heating is performed at a temperature suitable to inactivate pathogens, including viruses, prior to contacting the obtained virus-inactivated biological sample with the extraction composition in (B), optionally wherein heating is performed at a temperature ≥50° C., ≥55° C. or ≥60, preferably ≥75° C., ≥80° C. or ≥85° C., more preferably ≥90° C. or ≥95° C.
100. The method according to item 99, wherein (i) after heating, the virus-inactivated biological sample is contacted within ≤2 h, ≤1 h, ≤0.5 h, ≤20 min or ≤15 min with the extraction composition or wherein (ii) after heating the virus-inactivated biological sample is put on hold, stored or transported prior to contacting the pathogen heat-inactivated biological sample with the extraction composition.
101. The method according to any one of the preceding items, wherein virus-inactivation is assisted by heating and the method comprises heating the biological sample in the collection container used for receiving the collected biological sample, optionally wherein
(i) the biological sample is comprised in a medium in the collection container, wherein preferably, the medium comprises a virus-deactivating substance as defined in the preceding items;
(ii) the collection container has not been opened after collection of the biological sample and prior to heating.
102. The method according to any one of items 1 to 101, wherein subjecting at least an aliquot or all of the optionally pretreated virus-inactivated biological sample to an amplification reaction in (C) comprises contacting the optionally pretreated virus-inactivated biological sample with the components used for performing the amplification or reverse transcription amplification reaction thereby providing an amplification reaction admixture.
103. The method according to item 102, wherein the prepared amplification reaction admixture comprises:
(a) the optionally pretreated virus-inactivated biological sample;
(b) a DNA polymerase;
(c) optionally a reverse transcriptase, which is included in case a reverse transcription amplification is performed;
(d) an amplification reaction buffer comprising a Mg²⁺ source, a buffering agent and optionally further additives;
(e) nucleotides, preferably a dNTP mix, optionally wherein the nucleotides comprise modified nucleotides or dUTP; and
(f) primers for amplifying the one or more target nucleic acids and optionally probes.
104. The method according to item 103, wherein the method comprises contacting the optionally pretreated virus-inactivated biological sample with an amplification master mix comprising components (b) to (e) and separately provided primers for amplifying the one or more target nucleic acids and optionally probes.
105. The method according to item 103, wherein the method comprises contacting the optionally pretreated virus-inactivated biological sample with a direct amplification master mix comprising components (b) and (d) to (f) and optionally (c).
106. The method according to any one of items 103 to 105, wherein the optionally pretreated virus-inactivated biological sample comprises medium used for collecting the biological sample, wherein preferably, said medium is a medium as defined in any one of items 72 to 76, in particular items 72 to 74, and wherein the ionic strength of amplification reaction buffer (d), the amplification master mix comprising components (b) to (e) and/or the direct amplification master mix comprising components (b) to (f) is reduced to thereby compensate the introduction of ions, in particular ions derived from alkali metal salts and/or chlorides, into the amplification reaction admixture due to the prepared biological sample that contains the medium.
107. The method according to any one of items 103 to 106, wherein the amplification reaction buffer (d) has one or more of the following characteristics:
(aa) the amplification reaction buffer (d) does not comprise sodium chloride in a concentration ≥30 mM ≥20 mM ≥15 mM, ≥10 mM or ≥5 mM and wherein preferably, the amplification reaction buffer (d) contains no sodium chloride;
(bb) the amplification reaction buffer (d) does not comprise potassium chloride in a concentration ≥30 mM ≥20 mM ≥15 mM, ≥10 mM or ≥5 mM and wherein preferably, the amplification reaction buffer (d) contains no potassium chloride;
(cc) the amplification reaction buffer (d) does not comprise potassium chloride or sodium chloride;
(dd) the alkali metal chloride concentration in the amplification reaction buffer (d) is ≤30 mM, ≤20 mM, ≤15 mM or ≤10 mM and wherein preferably, the amplification reaction buffer (d) does not contain alkali metal chlorides; and
(ee) the alkali metal salt concentration in the amplification reaction buffer (d) is ≤30 mM, ≤20 mM, ≤15 mM or ≤10 mM and wherein preferably, the amplification reaction buffer (d) does not contain alkali metal salts.
108. The method according to any one of items 103 to 107, wherein the amplification reaction buffer (d) comprises a buffering agent that does not comprise chloride ions, optionally wherein the buffering agent is selected from the group consisting of tris(hydroxymethyl)aminomethane, N-(tri(hydroxymethyl)methyl)glycine, N,N-bis(2-hydroxyethyl)glycine, 3-(N-morpholino)propanesulphonic acid, N-(2-hydroxyethyl)piperazine-N′-(2-ethanesulphonic acid), piperazine-1,4-bis(2-ethanesulphonic acid), N-cyclohexyl-2-aminoethanesulphonic acid and 2-(N-morpholino)ethanesulphonic acid and preferably is selected from tris(hydroxymethyl)aminomethane and 3-(N-morpholino)propanesulphonic acid.
109. The method according to any one of items 103 to 108, wherein the pH of the amplification reaction buffer (d) is adjusted with an acid that does not comprise chloride, optionally wherein the pH is adjusted using an organic acid, preferably a carboxylic acid.
110. The method according to item 109, wherein the pH of the amplification reaction buffer (d) is adjusted with a carboxylic acid selected from acetic acid, formic acid, propionic acid and butyric acid and preferably is adjusted with acetic acid.
111. The method according to one or more of items 103 to 110, wherein the amplification reaction buffer (d) comprises a soluble magnesium salt as Mg²⁺ source that does not comprise chloride.
112. The method of item 111, wherein the soluble magnesium salt is a magnesium salt of an organic acid or wherein the magnesium salt is selected from magnesium sulfate and magnesium acetate.
113. The method according to any one of items 103 to 112, wherein the amplification reaction buffer (d) is characterized in that:
(i) it does not comprise potassium chloride or sodium chloride;
(ii) it comprises a buffering agent that does not comprise chloride ions, optionally wherein the buffering agent is selected from tris(hydroxymethyl)aminomethane and 3-(N-morpholino)-propanesulphonic acid, and
(iii) the pH of the amplification reaction buffer (d) is adjusted with an organic acid, preferably a carboxylic acid.
114. The method according to any one of items 103 to 113, when depending on item 105 or 106, wherein the amplification master mix comprising components (b) to (e) or the direct amplification master mix comprising components (b) to (f) has one or more of the following characteristics:
(aa) it does not comprise sodium chloride in a concentration ≥50 mM, ≥20 mM, ≥15 mM or ≥10 mM and wherein preferably, it contains no sodium chloride;
(bb) it does not comprise potassium chloride in a concentration ≥100 mM, ≥75 mM, ≥60 mM or ≥50 mM, optionally wherein it contains no potassium chloride;
(cc) it does not comprise potassium chloride or sodium chloride;
(dd) the alkali metal chloride concentration in the amplification master mix or the direct amplification master mix is ≤100 mM, ≤75 mM, ≤60 mM, ≤50 mM or ≤45 mM, optionally wherein it does not contain alkali metal chlorides;
(ee) wherein the alkali metal salt concentration in the amplification master mix or the direct amplification master mix is ≤100 mM, ≤75 mM, ≤60 mM, ≤50 mM or ≤45 mM; and/or
(ff) the chloride ion concentration is ≤250 mM, ≤200 mM, ≤175 mM or ≤150 mM.
115. The method according to any one of items 103 to 114, when depending on item 105 or 106, wherein the amplification master mix comprising components (b) to (e) or the direct amplification master mix comprising components (b) to (f) has one or both of the following characteristics:
(aa) it comprises a buffering agent that does not comprise chloride ions, optionally wherein the buffering agent is selected from the group consisting of tris(hydroxymethyl)aminomethane, N-(tri(hydroxymethyl)methyl)glycine, N,N-bis(2-hydroxyethyl)glycine, 3-(N-morpholino)propanesulphonic acid, N-(2-hydroxyethyl)piperazine-N′-(2-ethanesulphonic acid), piperazine-1,4-bis(2-ethanesulphonic acid), N-cyclohexyl-2-aminoethanesulphonic acid and 2-(N-morpholino)ethanesulphonic acid and preferably is selected from tris(hydroxymethyl)aminomethane and 3-(N-morpholino)propanesulphonic acid;
(bb) the pH is adjusted with an organic acid, preferably a carboxylic acid, optionally wherein the carboxylic acid selected from acetic acid, formic acid, propionic acid and butyric acid, preferably acetic acid.
116. The method according to any one of items 103 to 115, when depending on item 105 or 106, wherein the amplification master mix comprising components (b) to (e) or the direct amplification master mix comprising components (b) to (f) comprises a soluble magnesium salt as Mg²⁺ source that does not comprise chloride, optionally wherein the soluble magnesium salt is a magnesium salt of an organic acid, preferably selected from magnesium sulfate and magnesium acetate.
117. The method according to any one of items 103 to 116, when depending on item 105 or 106, wherein the amplification master mix comprising components (b) to (e) is characterized in that:
(i) it does not comprise sodium chloride in a concentration ≥50 mM, ≥20 mM, ≥15 mM or ≥10 mM and wherein preferably, it contains no sodium chloride;
(ii) it does not comprise potassium chloride in a concentration ≥100 mM, ≥75 mM, ≥60 mM or ≥50 mM, optionally wherein it contains no potassium chloride;
(iii) if chloride ions are present, the chloride ion concentration is ≤250 mM, ≤200 mM, ≤175 mM, ≤150 mM or ≤125 mM;
(iv) it comprises a buffering agent that does not comprise chloride ions, wherein preferably the buffering agent is selected from tris(hydroxymethyl)aminomethane and 3-(N-morpholino)-propanesulphonic acid;
(v) optionally wherein the pH is adjusted with an acid that does not comprise chloride, preferably an organic acid, more preferably a carboxylic acid, optionally wherein the carboxylic acid is acetic acid.
118. The method according to one or more of items 103 to 117, wherein the amplification buffer (d), the amplification master mix comprising components (b) to (e) or the direct amplification master mix comprising components (b) to (f) comprises one or more of the following additives:
   an ammonium salt, optionally selected from ammonium sulfate and ammonium chloride;
   polyethylene glycol;
   N,N,N-trimethylglycine;
   serum albumin;
   a metal ion chelator, optionally EGTA;
   glycerol;
   fish gelatine;
   PVP (polyvinylpyrrolidone);
   DMSO; and
   formamide.
119. The method according to one or more of items 103 to 118, wherein the prepared amplification reaction admixture comprises:
(g) at least one internal control template and primers for amplifying said internal control template and optionally probes for detection.
120. The method according to any one of items 1 to 119, wherein the optionally pretreated virus-inactivated biological sample that is subjected to the amplification reaction provides at least 20%, at least 30%, at least 40% or at least 45% of the total reaction volume of the amplification reaction, optionally wherein the optionally pretreated virus-inactivated biological sample that is subjected to the amplification reaction provides up to 60% or up to 50% of the total reaction volume of the amplification reaction, which preferably is a reverse transcription PCR or PCR.
121. The method according to any one of items 1 to 120, wherein the amplification reaction has one or more of the following characteristics (i) it is a reverse transcription amplification reaction; (ii) it is a reverse transcription PCR; (iii) it is an isothermal amplification reaction; (iv) it is a polymerase chain reaction (PCR); (v) it is a quantitative PCR; (vi) it is a quantitative reverse transcription PCR; (vii) it is a digital PCR.
122. The method according to any one of items 1 to 121, wherein the amplification reaction is a reverse-transcription amplification reaction, preferably a quantitative reverse-transcription polymerase chain reaction.
123. The method according to any one of items 77 to 122, wherein the steps of
   contacting the virus-inactivated biological sample with the extraction composition,
   incubating the admixture to provide the pretreated virus-inactivated biological sample, and
   performing the amplification reaction, preferably a reverse-transcription amplification reaction, are performed within the same reaction vessel.
124. The method according to any one of items 1 to 123, wherein (i) providing in (A) and pretreating in (B) are performed concurrently, (ii) providing in (A) and subjecting in (C) are performed concurrently, or (iii) providing in (A), pretreatment in (B) and subjecting in (C) are performed concurrently.
125. The method according to any one of items 1 to 124, wherein the biological sample is a respiratory biological sample and the virus is a RNA virus, wherein the method comprises
(A) providing a virus-inactivated biological sample, wherein providing such sample comprises preparing a composition comprising the biological sample and at least one virus-deactivating substance, and wherein providing comprises
   contacting the biological sample with at least one virus-deactivating substance; wherein the virus-deactivating substance is selected from the group consisting of oxidizing agents, cationic surfactants, non-ionic surfactants, and zwitterionic surfactants; and
   incubating the composition to provide the virus-inactivated biological sample;
(B) optionally pretreating the biological sample, wherein optionally pretreating comprises
   contacting an aliquot or all of the biological sample with an extraction composition as defined in any one of item 77 to 96, thereby providing an admixture; and
   incubating the admixture to provide the pretreated virus-inactivated biological sample;
and
(C) subjecting at least an aliquot or all of the optionally pretreated virus-inactivated biological sample to a reverse transcription and amplification reaction, which preferably is a quantitative RT-PCR reaction, wherein the optionally pretreated virus-inactivated biological sample is in contact with the components used for performing the reverse transcription amplification reaction thereby providing an amplification reaction admixture, wherein the prepared amplification reaction admixture comprises
   (a) the optionally pretreated virus-inactivated biological sample;
   (b) a DNA polymerase;
   (c) a reverse transcriptase;
   (d) an amplification reaction buffer comprising a Mg²⁺ source, a buffering agent and optionally further additives;
   (e) nucleotides, preferably a dNTP mix; and
   (f) primers for reverse transcribing and amplifying the one or more target nucleic acids,
   and performing the reverse transcription and amplification reaction to reverse transcribe and amplify at least one RNA target nucleic acid derived from the RNA virus.
126. The method according to item 125, wherein pretreatment step (B) is performed and wherein in (C) the pretreated virus-inactivated biological sample provides at least 20%, at least 30% or at least 40% of the total reaction volume of the prepared amplification reaction admixture; and
wherein at least the steps of
   contacting the biological sample with the extraction composition to prepare the admixture,
   incubating the admixture, and
   performing the reverse-transcription amplification reaction,
are performed within the same reaction vessel,
and wherein the target nucleic acid is provided by one or more, preferably two or more, target nucleic acids derived from a severe acute respiratory syndrome-related coronavirus, preferably severe acute respiratory syndrome coronavirus 2 (SARS-CoV-2).
127. The method according to any one of items 1 to 126, wherein steps (B) and (C) are completed in 2 h or less, 1.5 h or less or 1 h or less.
128. A kit for performing a method as defined in any one of items 1 to 127, comprising
(a) a virus-deactivating substance as defined in any one of the preceding items; and one or more and preferably all of the following components:
(b) a DNA polymerase;
(c) a reverse transcriptase;
(d) an amplification reaction buffer comprising a Mg²⁺ source, a buffering agent and optionally further additives;
(e) nucleotides, preferably a dNTP mix; and
(f) primers for amplifying the at least one target nucleic acid, optionally wherein components (b) to (e) or (b) to (f) are comprised in a single composition.
129. The kit according to item 128, wherein the kit comprises
(g) an extraction composition as defined in any one of items 77 to 96.
130. The kit according to item 128 or 129, wherein the amplification buffer (d) is as defined in any one of items 107 to 113.
131. The kit according to any one of items 128 to 130, wherein components (b) to (e) are comprised in a single composition thereby providing an amplification master mix.
132. The kit according to any one of items 128 to 130, wherein components (b) to (f) are comprised in a single composition thereby providing a direct amplification master mix.
133. The kit according to item 131 or 132, wherein the amplification master mix comprising components (b) to (e) or the direct amplification master mix comprising components (b) to (f) is as defined in any one of items 114 to 117.
134. The kit according to any one of items 128 to 133, wherein the amplification buffer (d), the amplification master mix comprising components (b) to (e) or the direct amplification master mix comprising components (b) to (f) comprises one or more of the following additives:
   an ammonium salt, optionally selected from ammonium sulfate and ammonium chloride;
   polyethylene glycol;
   N,N,N-trimethylglycine;
   serum albumin;
   a metal ion chelator, optionally EGTA;
   glycerol;
   fish gelatin;
   PVP (polyvinylpyrrolidone);
   DMSO; and
   formamide.
135. The kit according to any one of items 128 to 134, wherein the kit comprises
(h) at least one internal control template and primers for amplifying said internal control template and optionally probes for detection.
136. The kit according to any one of items 128 to 134, wherein the kit comprises at least one substance suitable to counteract the inhibitory effect of the at least one virus-deactivating substance on the activity of the DNA polymerase and/or the reverse transcriptase.
137. The kit according to item 136, wherein the substance suitable to counteract the inhibitory effect of the at least one virus-deactivating substance is a surfactant or a reducing agent.
138. Use of a kit according to any one of items 128 to 137 in a method as defined in any one of items 1 to 127.
139. Use of a virus-deactivating substance for preparing a virus-inactivated biological sample for use in the direct amplification of target nucleic acids comprised in the biological sample without prior target nucleic acid purification, wherein preferably, the amplification reaction is a reverse transcription amplification reaction.
140. Use according to item 139, wherein the virus-deactivating substance is comprised in a liquid composition that is contacted with the biological sample to provide the virus-inactivated biological sample.
141. Use according to item 140, wherein the liquid composition comprising the virus-deactivating substance is a medium for storing or transporting the biological sample.
142. Use according to any one of items 139 to 141, wherein the use comprises pretreating the virus-inactivated biological sample prior to performing the amplification reaction, which preferably is a is reverse transcription amplification reaction.
143. Use according to any one of items 139 to 142, wherein the virus-deactivating substance is used in combination with a further substance suitable to counteract the inhibitory effect of the at least one virus-deactivating substance on the activity of the DNA polymerase and/or the reverse transcriptase used in the amplification reaction, optionally wherein the substance suitable to counteract the inhibitory effect of the at least one virus-deactivating substance is a surfactant or a reducing agent.
144. Use according to any one of items 139 to 143, having one or more of the following features:
(a) the virus-deactivating substance is as defined in any one of items 16 to 63;
(b) for pretreatment of the virus-inactivated biological sample, an extraction composition as defined in any one of items 77 to 96 is used;
(c) the use comprises performing the method according to any one of items 1 to 127.

As demonstrated in the examples, the method according to the invention using a virus-deactivating substance as disclosed herein is capable of providing constant Ct-values in PCR and RT-PCR applications for detecting the presence of absence of a virus, such as a RNA virus, such as SARS-CoV-2, in a biological sample, including respiratory specimens. This demonstrates that the viral RNA is stable and not degraded in spite of the virus inactivation technology according to the invention.

### The Kit According to the Second Aspect

According to the second aspect, a kit for performing the method according to the first aspect is provided. Said kit comprises:
a) a virus-deactivating substance as defined in any one of the preceding embodiments; and one or more and preferably all of the following components:
(b) a DNA polymerase;
(c) optionally a reverse transcriptase;
(d) an amplification reaction buffer comprising a Mg²⁺ source, a buffering agent and optionally further additives;
(e) nucleotides, preferably a dNTP mix; and
(f) primers for amplifying the at least one target nucleic acid, optionally wherein components (b) to (e) or (b) to (f) are comprised in a single composition.

The kit may furthermore comprise
(g) an extraction composition as defined in any one of the embodiments above.

According to one embodiment, the kit comprises an amplification buffer (d) as disclosed in conjunction with the method according to the first aspect and it is referred to the respective disclosure. According to one embodiment, components (b) to (e) are comprised in a single composition thereby providing an amplification master mix. According to one embodiment, components (b) to (f) are comprised in a single composition thereby providing a direct amplification master mix. The kit may comprise an amplification master mix comprising components (b) to (e) or a direct amplification master mix comprising components (b) to (f) as disclosed in conjunction with the method according to the first aspect and it is referred to the respective disclosure.

According to one embodiment, the amplification buffer (d), the amplification master mix comprising components (b) to (e) or the direct amplification master mix comprising components (b) to (f) comprises one or more of the following additives:
an ammonium salt, optionally selected from ammonium sulfate and ammonium chloride;
polyethylene glycol;
N,N,N-trimethylglycine;
serum albumin;
a metal ion chelator, optionally EGTA;
glycerol;
fish gelatin;
PVP (polyvinylpyrrolidone);
DMSO; and
formamide.

According to one embodiment, the kit comprises
(h) at least one internal control template and primers for amplifying said internal control template and optionally probes for detection.

According to one embodiment, the kit furthermore comprises at least one substance suitable to counteract the inhibitory effect of the at least one virus-deactivating substance on the activity of the DNA polymerase and/or the reverse transcriptase. According to an advantageous embodiment, the substance suitable to counteract the inhibitory effect of the at least one virus-deactivating substance is a surfactant or a reducing agent. Substances suitable to counteract the inhibitory effect of different virus-deactivating substances and combinations of specific virus-deactivating substances and counteracting substances were described above in conjunction with the method and are also disclosed in the examples. It is referred to the corresponding disclosure which also applies in conjunction with the kit.

### Uses According to the Third and Fourth Aspects

In a third aspect, the present invention relates to the use of a kit according to the second aspect in the method according to the first aspect. Details of the respective kit and the methods are described in detail above and it is referred to the respective disclosure which also applies here.

In a fourth aspect, the present invention relates to the use of a virus-deactivating substance for preparing a virus-inactivated biological sample for use in the direct amplification of target nucleic acids comprised in the biological sample without prior target nucleic acid purification, wherein preferably, the amplification reaction is a reverse transcription amplification reaction. According to one embodiment the virus-deactivating substance is comprised in a liquid composition that is contacted with the biological sample to provide the virus-inactivated biological sample. According to an advantageous embodiment, the liquid composition comprising the virus-deactivating substance is a medium for storing or transporting the biological sample.

According to one embodiment, the use according to the fourth aspect comprises pretreating the virus-inactivated biological sample prior to performing the amplification reaction, which preferably is a reverse transcription amplification reaction. Suitable embodiments for pretreatment were described in detail in conjunction with the method according to the present invention and it is referred to the corresponding disclosure. An extraction composition as disclosed herein, which preferably is an extraction solution, can be used for pretreatment. As disclosed herein, the technology of the present disclosure has the advantage that at least 20%, at least 30%, at least 40% or at least 45% of the total reaction volume of the amplification reaction can be provided by the optionally pretreated virus-inactivated biological sample that is subjected to the amplification reaction. In embodiments, it provides up to 60% or up to 50% of the total reaction volume of the amplification reaction, which preferably is a reverse transcription PCR or PCR.

According to one embodiment, the virus-deactivating substance is used in combination with a further substance suitable to counteract the inhibitory effect of the at least one virus-deactivating substance on the activity of the DNA polymerase and/or the reverse transcriptase used in the amplification reaction, optionally wherein the substance suitable to counteract the inhibitory effect of the at least one virus-deactivating substance is a surfactant or a reducing agent.

According to one embodiment, the use according to the fourth aspect having one or more of the following features:
(a) the virus-deactivating substance is as defined in any one of the embodiments above;
(b) for pretreatment of the virus-inactivated biological sample, an extraction composition as defined in any one of the embodiments above is used;
(c) the use comprises performing the method according to any one of the embodiments above.

According to a fifth aspect the present disclosure pertains to the use of the method according to the first aspect for the detection of pathogens other than viruses.

As is apparent from the above disclosure and the examples provided below, the present invention provides a streamlined workflow for providing virus-inactivated biological samples for amplification based detection of target nucleic acids and viruses without prior nucleic acid purification which inter alia enables an immediate and fast real-time PCR run. The present invention enables a straightforward workflow. A primary sample, such as a nasopharyngeal, an oropharyngeal or a nasal swab, optionally comprised in transport media, such as Universal Transport Media (UTM™), is contacted with a virus-deactivating substance of the present invention to provide a virus-inactivated biological sample. This virus inactivation technology allows the handling of the sample under standard biosafety conditions circumventing the need for time- and cost-intensive preventive measures. Subsequently to virus inactivation, the virus-inactivated biological sample optionally is pretreated by contacting the virus-inactivated biological sample to the extraction solution of the present invention that is particularly suitable to pretreat viral nucleic acids, including viral RNA, without degradation. Furthermore, the extraction composition of the invention may comprise substances that counteract inhibitory effects on reverse transcription and/or amplification reactions of the virus-deactivating substances, thereby overcoming the drawbacks of prior art virus inactivation methods. The admixture comprising the optionally pretreated virus-inactivated biological sample comprising the virus-deactivating substance of the invention, transport media and the extraction solution of the invention is then combined with the components of the amplification reaction, which preferably is a reverse transcription amplification. As disclosed herein, a routine real-time PCR can be performed using the optionally pretreated virus-inactivated biological sample without prior purification which provides reliable and sensitive results. Advantageously any cycler can be used and the overall rapid workflow of the invention allows to deliver results in under one hour. Thus, the present invention significantly simplifies and accelerates PCR analysis compared to standard extraction-based quantitative PCR processes, which e.g. require three hours and more to obtain a result. This enables laboratories to significantly increase the frequency of virus tests. The level of detection that can be achieved with the method of the present invention is similar to or better than regular PCR workflows and its performance compares to standard public health protocols of the U.S. Centers of Disease Control (CDC), the World Health Organization (WHO) and others that use the gold standard for sample extraction. Furthermore, the present invention is compatible with standard laboratory automation equipment, standard assay and transport media and allows to combine the reagents for sample preparation and target detection in one kit. Furthermore, significant cost savings are possible by reducing plastic and reagent use as well as laboratory utilization. Overall, the present invention removes key testing bottlenecks for virus detection, such as SARS-CoV-2 and other RNA viruses by providing a safe, accelerated and simplified workflow that does not require nucleic acid purification.

The present application claims priority of the following applications EP 21 158 281.2 of Feb. 19, 2021, EP 21 158 283.8 of Feb. 19, 2021, EP 20 200 426.3 of Oct. 6, 2020, EP 20 200 425.5 of Oct. 6, 2020, U.S. 63/088,423 of Oct. 6, 2020 and EP 20 214 412.7 of Dec. 16, 2020 the content of which is herein incorporated by reference.

### EXAMPLES

It should be understood that the following examples are for illustrative purpose only and are not to be construed as limiting this invention in any manner.

The following examples demonstrate the superior performance of the direct PCR system using virus-inactivated biological samples according to the present invention.

### Abbreviations

UTM Universal Transport Medium
VTM Viral Transport Medium
DDAC Didecyldimethylammonium chloride
LDAO Lauryldimethylamin-N-oxide (N,N-Dimethyldodecylamine N-oxide)
NaCl sodium chloride
SDS Sodium dodecyl sulfate
TCEP Tris-(2-carboxyethyl)-phosphin
RT reverse transcriptase
PCR polymerase chain reaction
IC internal control
NADB Nucleic Acid Dilution Buffer (QIAGEN, Hilden)

If not otherwise mentioned, experiments were performed either with the QuantiNova Pathogen+IC Kit (QIAGEN, Hilden) or the QIAprep&amp Viral RNA UM Kit (QIAGEN, Hilden) as indicated in the respective examples, both herein incorporated by reference. Table 1 presents the PCR components used for preparing the PCR reaction admixture comprised in the QuantiNova Pathogen Master Mix, as is also indicated in the QuantiNova Pathogen+IC Kit Handbook (QIAGEN, May 2016), and in the QIAprep&amp Viral RNA UM Master Mix, as indicated in the QIAprep&amp Viral RNA UM Kit Handbook (QIAGEN, November 2020), respectively:

TABLE 1
Core components of the QuantiNova Pathogen Master Mix
as indicated in the QuantiNova Pathogen + IC Kit
Handbook (QIAGEN, May 2016) and the QIAprep&amp Viral
RNA UM Master Mix as indicated in the QIAprep&amp Viral
RNA UM Kit Handbook (QIAGEN, November 2020), respectively

| Kit | Component | Description |
|---|---|---|
| QuantiNova | QuantiNova DNA | Modified Taq polymerase |
| Pathogen + | Polymerase | (recombinant 94 kDa DNA |
| IC Kit | | polymerase) |
| | HotStartRT-Script | Modified form of a recombinant |
| | Reverse Transcriptase | 77 kDa reverse transcriptase |
| | QuantiNova Pathogen | Tris-HCl |
| | Buffer (also referred to | KCl |
| | herein as QN reaction | NH₄Cl |
| | buffer or QN PCR | MgCl₂ |
| | buffer) | additives enabling fast |
| | | cycling |
| | dNTP mix | dATP, dCTP, dGTP, dTTP |
| QIAprep&amp | QuantiNova DNA | Modified Taq polymerase |
| Viral RNA | Polymerase | (recombinant 94 kDa DNA |
| UM Kit | | polymerase) |
| | HotStartRT-Script | Modified form of a recombinant |
| | Reverse Transcriptase | 77 kDa reverse transcriptase |
| | Buffer | components enabling fast |
| | | cycling, including Q-Bond |
| | dNTP mix | dATP, dCTP, dGTP, dTTP |

Unless indicated otherwise in the below examples, the amplification protocol of the QuantiNova Pathogen+IC Kit as it is disclosed in the 2016 Handbook (QIAGEN, Hilden) or of the QIAprep&amp Viral RNA UM Kit as it is disclosed in the 2020 Handbook (QIAGEN, Hilden) was followed. All reaction volumes were 20 μl in total following the manufacturer's instructions. All virus-deactivating substances were mixed with the transport medium UTM in a given concentration (see examples below) and 7 μl of this mixture were applied to the amplification reaction resulting in a final concentration as given in the examples. Amplification took place for 40 or 45 cycles as indicated in the respective examples.

To distinguish inhibitory effects on the different enzymes by the test substances all substances were tested with RNA and DNA templates for effects on reverse transcriptase (RT) and Taq polymerase as example of a common DNA polymerase, respectively.

For the Taq polymerase, if not otherwise mentioned, human genomic DNA was used as template material with the IC assay from the Investigator QuantiPlex Pro Kit (QIAGEN, Hilden). Respective Ct-values are presented as grey bars in the diagrams shown in the examples below. In case of the reverse transcription (RT), Internal Control RNA from the QuantiNova Pathogen Kit (QN IC RNA; QIAGEN, Hilden) was used. Corresponding Ct-values are shown as black bars in the diagrams shown in the examples below.

All targets and assays are listed in the respective experiment and suitable primers were included in the PCR reaction admixture to allow amplification of the targets. As target heat-inactivated virus particles (SARS-CoV-2; Zeptometrix, Buffalo NY, Cat-No. NATSARS(COV2)-ST) were used as mentioned in the respective example. SARS-CoV-2 Assays performed in the following experiments rely on target sequences for the SARS-CoV-2 genes N1 and N2 published by the US CDC (htts://www.cdc.gov/coronavirus/2019-ncov/lab/rt-pcr-panel-primer-probes.html, last updated May 29, 2020).

Virucidal activity of the test substances was demonstrated for the Vaccinia virus, a DNA virus, and the Bovine coronavirus (Betacoronavirus 1), a RNA virus. Analysis was performed by Henkel (Dusseldorf) according to the European norm DIN EN 14476:2019-10.

### Example 1: Estimation of Non-Inhibiting Concentrations of Neutral and Non-Ionic Surfactants

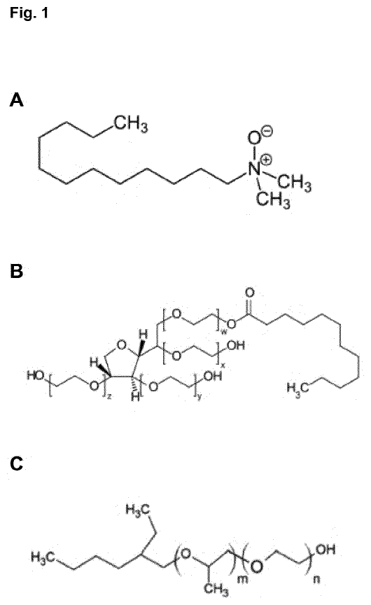

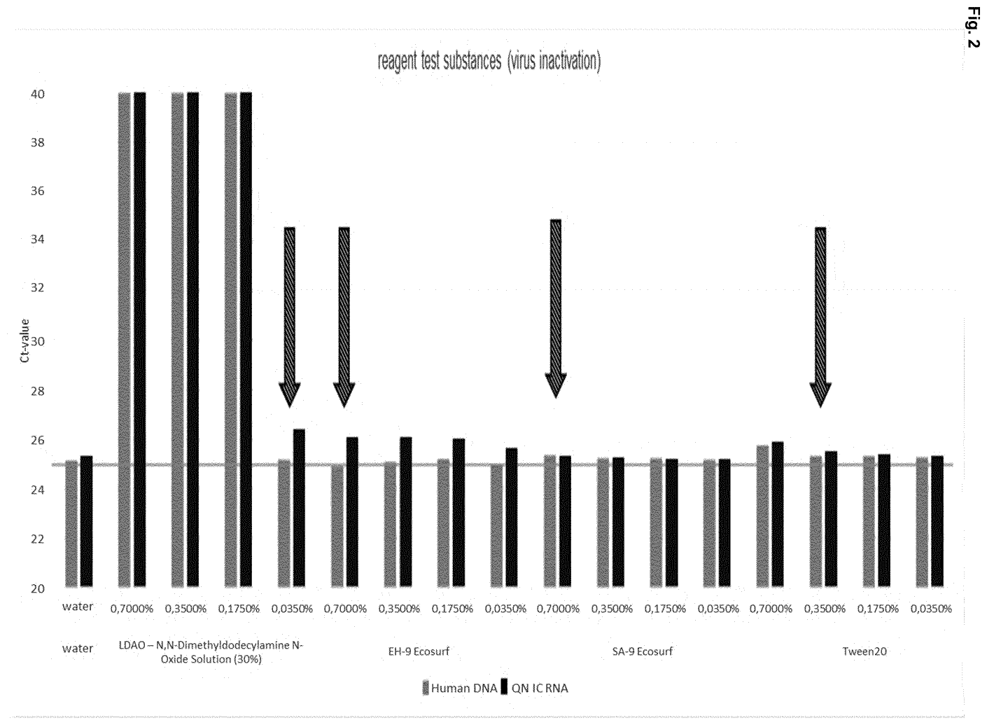

A number of non-ionic and neutral (zwitterionic) surfactants were tested to determine the maximal non-inhibiting concentrations in (RT-)PCR (FIG. 1). In detail, the inhibitory effect of Lauryldimethylamin-N-oxide (N,N-Dimethyldodecylamine N-oxide, LDAO), Ecosurf EH-9, Ecosurf SA-9, and Tween20 on the amplification reaction was investigated in this example. The following concentrations in the transport medium (UTM) and the final concentrations in the (RT-)PCR reactions of the tested surfactants were used:

### Concentrations in UTM (%):

LDAO (w/w): 2.0/1.0/0.5/0.1
Ecosurf EH-9 (v/v): 2.0/1.0/0.5/0.1
Ecosurf SA-9 (v/v): 2.0/1.0/0.5/0.1
Tween20 (v/v): 2.0/1.0/0.5/0.1

### Final Concentrations in (RT-)PCR Reactions (%):

LDAO (w/w): 0.7/0.35/0.175/0.035
Ecosurf EH-9 (v/v): 0.7/0.35/0.175/0.035
Ecosurf SA-9 (v/v): 0.7/0.35/0.175/0.035
Tween20 (v/v): 0.7/0.35/0.175/0.035

(RT-)PCR war performed with the QuantiNova Pathogen Mix (QIAGEN) for 40 cycles. The substances were added in decreasing concentrations to the (RT-)qPCR. Water was used as control. The underlined highlighted concentrations indicate the maximal non-inhibiting concentrations according to FIG. 2 (see arrows) tested in this experiment.

### Results:

As shown in FIG. 2, LDAO shows a slight but acceptable inhibition of the RT reaction (black bars) and no inhibition of the Taq polymerase (grey bars) only at the lowest tested concentration of 0.035% compared to water control, which is comparable to the results obtained with Ecosurf EH-9 at all tested concentrations. For Ecosurf SA-9 no inhibition of the RT or Taq polymerase was observed for all tested concentrations. Furthermore, no enzyme inhibition was detected by the use of Tween20 from tested concentrations down from 0.35%. At 0.7%, a some inhibition was seen with Tween20.

Therefore, these substances are suitable to be used in the preparation of a virus-inactivating biological sample that can be directly used in an amplification reaction, including a reverse transcription amplification, without prior purification of the comprised nucleic acids.

### Example 2: Estimation of Non-Inhibiting Concentrations of Other Virus-Deactivating Substances

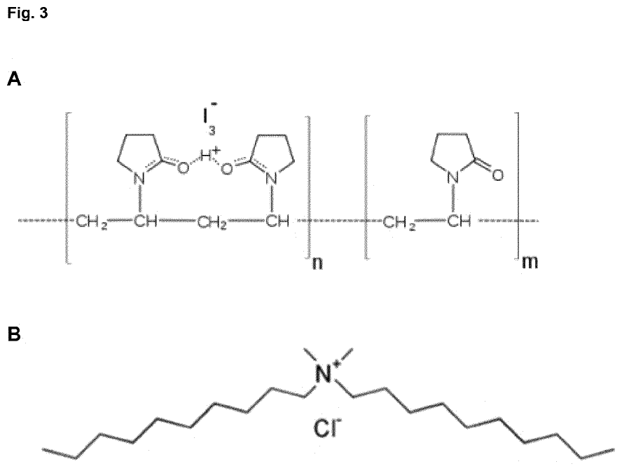

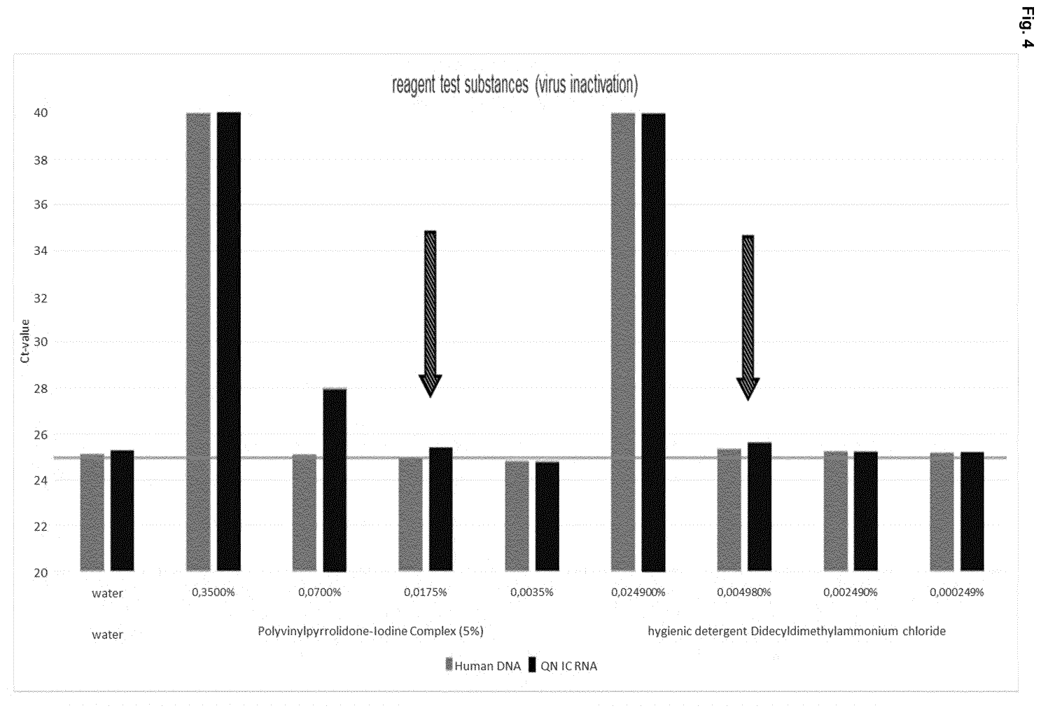

In addition to non-ionic and neutral surfactants, other virus-deactivating substances were tested for their compatibility with the (RT-)qPCR, here a disinfectant and a cationic surfactant. Povidone iodine was used as exemplary disinfectant and a quaternary ammonium compound, namely Didecyldimethylammonium chloride (DDAC), as cationic surfactant (FIG. 3).

Both substances were added in decreasing concentrations to the (RT-)qPCR and the Ct-values were compared to a water control to detect inhibitory effects.

### Concentrations in UTM (%):

Povidone iodine (w/w): 1.0/0.2/0.05/0.01
DDAC (w/w): 0.071/0.014/0.007/0.0007

### Final Concentrations in RT-qPCR Reactions (%):

Povidone iodine (w/w): 0.35/0.07/0.0175/0.0035
DDAC (w/w): 0.025/0.005/0.0025/0.00025

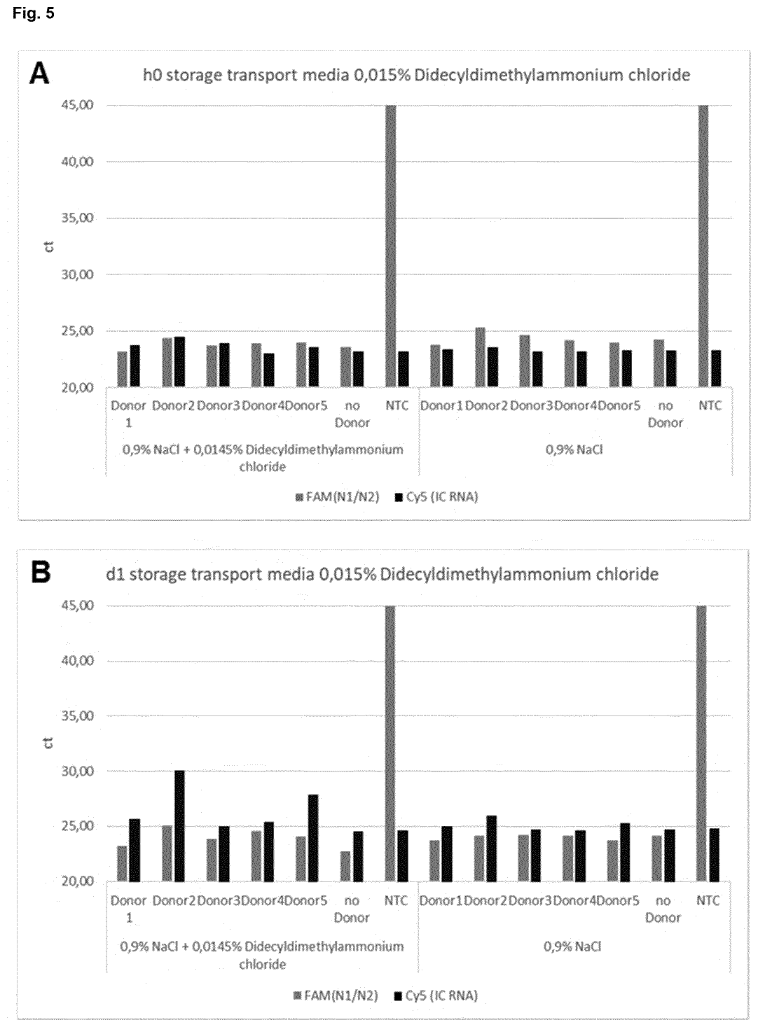

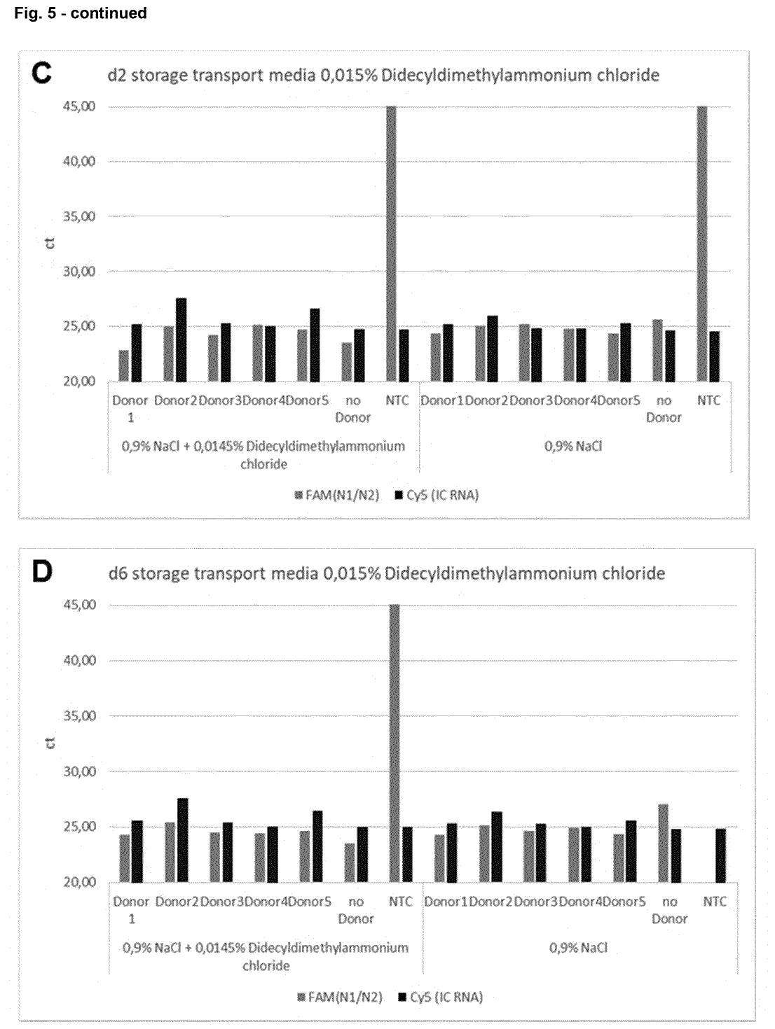

(RT-)PCR war performed with the QuantiNova Pathogen Mix (QIAGEN) for 40 cycles. The underlined concentrations indicate the maximal non-inhibiting concentrations according to FIG. 4 (see arrows) tested in this experiment.

### Results:

As shown in FIG. 4, povidone iodine shows no inhibition of both RT (black bars) and Taq polymerase (grey bars) if 0.0175% or less is applied to the reaction compared to the water control. For DDAC no enzyme inhibition was observed with 0.005% or less.

Furthermore, and as shown below, it is also possible to use higher concentrations of these virus-deactivating substances in the amplification reaction, when additionally including a substance that counteracts the inhibitory effect of the virus-deactivating substance in the amplification reaction. As shown below, this allows to use higher concentrations of the virus-deactivating substance for virus-inactivation (thereby improving the virus-inactivation) while maintaining the performance of the amplification reaction.

### Example 3: Demonstration of Virucidal Activity for Vaccinia Virus

The following test for virucidal activity of the different test substances were performed by Henkel (Dusseldorf) according to the European norm DIN EN 14476:2019-10. According to the recommendations of this standard method the Vaccinia virus is used as model virus. Vaccinia virus is a model virus for enveloped viruses.

The concentrations of the substances tested for virus-inactivation predominantly correspond to the highest concentrations in UTM for which no inhibition in the (RT-)qPCR was observed (see Examples 1 and 2 above). Ecosurf EH-9 and SA-9 were used at lower concentrations (1% as compared to 2% in Example 1).

The experimental conditions were as follows:

### Test Concentration(s)

Tween20 active substance in test 1%
Ecosurf EH-9 active substance in test 1%
Ecosurf SA-7 active substance in test 1%
Ecosurf SA-9 active substance in test 1%
DDAC active substance in test 0.015%
Braunol povidone iodine active substance in test 0.05%
   Diluent WSH acc. EN14476

### Appearance of the Test Product Solution(s)

Tween20 1%×1.25=clear, colorless, liquid, no precipitation
Ecosurf EH-9 1%×1.25=clear, colorless, liquid, no precipitation
Ecosurf SA-7 1%×1.25=clear, colorless, liquid, no precipitation
Ecosurf SA-9 1%×1.25=clear, colorless, liquid, no precipitation
DDAC 0.015%×1.25=clear, colorless, liquid, no precipitation
Braunol povidone iodine 0.05%×1.25=clear, orange, liquid, no precipitation

### Appearance of Product(s) Plus Load and Virus-Suspension

Tween20 1%=clear, colorless, liquid, no precipitation
Ecosurf EH-9 1%=clear, colorless, liquid, no precipitation
Ecosurf SA-7 1%=clear, colorless, liquid, no precipitation
Ecosurf SA-9 1%=clear, colorless, liquid, no precipitation
DDAC 0.015%=clear, colorless, liquid, no precipitation
Braunol povidon iodine 0.05%=clear, colorless, liquid, no precipitation

### Test Organisms

Vaccinia virus, Elstree strain, ATCC VR-1549 obtained ATCC in 2020,
Passage 2 from 2020-05-14

### Host Cells

Vero-B4-Zellen AC 33, obtained from DSMZ in 2010, Passage 75 from 2020-07-27

### Contact Time(s)

0 minutes, 15 minutes, 60 minutes

### Test Temperature(s)

20° C.±1° C.

### Interfering Substance(s)

0.3 g/L BSA (=clean conditions)

### Procedure Prior the Test

All assay ingredients were allowed to adjust to test temperature. The assay was performed in a temperature-controlled water bath. For the titer determination of the virus inoculum preparation blanks were done with WSH instead of test product. After the contact time the reaction is annulled by transfer of 100 μl aliquots to 900 μl ice-cold FCS-supplemented DMEM and serial 1:10-dilutions up to 10-9. As annulment verification this is already controlled at time 0. The recovery of residual active virus was performed in quantal tests on the corresponding host cells in microtiter plates using 8 parallels by transferring 100 μl aliquots of each dilution to confluent monolayer cells. After readout for cytopathic effects (CPEs) with an inverse microscope the effects are evaluated as follows:
0=no cell damage=no virus activity
1=<25% cell damage=virus activity
2≈50% cell damage=virus activity
3≈75% cell damage=virus activity
4≈100% cell damage=virus activity

### Neutralization

Instantaneous dilutions of the test assays in ice-cold FCS-supplemented DMEM (1:10, 1:100

TABLE 2
Reagents used in the virucidal activity test.

| Reagent | Supplier | Batch/LOT | Best before |
|---|---|---|---|
| BSA (bovine serum albumine) | Serva | 200084 | October 2023 |
| DMEM | BioSell | BS.FG.04155P | February 2022 |
| FCS (Fetal Calf Serum) No. S-10-L | c-c.pro | G174 | October 2021 |
| Formol (as 37% formaldehyde) | Applichem | 1642934 | December 2024 |
| 10 × PBS | VWR | 19D1856448 | December 2020 |
| Trypsine/EDTA | Biowest | MS00EK | November 2021 |
| Distilled water | HSA-Microbiology | 2020060408-1 | December 2020 |
| WSH | HSA-Microbiology | 2020072801-1 | pH 6.92 |
| D-PBS | Hyclone | AF29477517 | May 2020 |

The virucidal activity tests revealed the following results after 0, 15 or 60 minutes. The reduction factor of the titer is given in log₁₀.

TABLE 3
Results of test for virucidal activity after
a residence time of 0 minutes (t = 0).

| | | | log₁₀ reduction factor(s) | |
|---|---|---|---|---|
| | | | incl. 95% confidence | |
| | | | interval(s) against | |
| | | | Vaccinia Virus, Elstree | |
| Parameter | | | strain, ATCC VR1549 | |
| | application | contact | temperature | With clean |
| product | conc. | time | (° C.) | conditions |
| Tween20 | 1% | 0 | 20 | 2.00 ± 0.54 |
| Ecosurf EH-9 | 1% | 0 | 20 | 2.38 ± 0.45 |
| Ecosurf SA-7 | 1% | 0 | 20 | 2.50 ± 0.38 |
| Ecosurf SA-9 | 1% | 0 | 20 | 2.75 ± 0.50 |
| DDAC | 0.015% | 0 | 20 | 1.25 ± 0.50 |
| Braunol | 0.05% | 0 | 20 | 3.00 ± 0.54 |
| povidone iodine | | | | |

| | | | | Titer (log₁₀) |
|---|---|---|---|---|
| | application | contact | temperature | with clean |
| control | conc. | time | (° C.) | conditions |
| WSH | — | 0 | 20 | 6.00 ± 0.38 |

TABLE 4
Results of tests for virucidal activity after
a residence time of 15 minutes (t = 15).

| | | | log₁₀ reduction factor(s) | |
|---|---|---|---|---|
| | | | incl. 95% confidence | |
| | | | interval(s) against | |
| | | | Vaccinia Virus, Elstree | |
| Parameter | | | strain, ATCC VR1549 | |
| | application | contact | temperature | With clean |
| product | conc. | time | (° C.) | conditions |
| Tween20 | 1% | 15 | 20 | 2.00 ± 0.54 |
| Ecosurf EH-9 | 1% | 15 | 20 | 2.25 ± 0.44 |
| Ecosurf SA-7 | 1% | 15 | 20 | 2.38 ± 0.37 |
| Ecosurf SA-9 | 1% | 15 | 20 | 3.25 ± 0.44 |
| DDAC | 0.015% | 15 | 20 | 2.63 ± 0.49 |
| Braunol | 0.05% | 15 | 20 | 4.00 ± 0.52 |
| povidone iodine | | | | |

| | | | | Titer (log₁₀) |
|---|---|---|---|---|
| | application | contact | temperature | with clean |
| control | conc. | time | (° C.) | conditions |
| WSH | — | 15 | 20 | 5.88 ± 0.37 |

TABLE 5
Results of tests for virucidal activity after
a residence time of 60 minutes (t = 60).

| | | | log₁₀ reduction factor(s) | |
|---|---|---|---|---|
| | | | incl. 95% confidence | |
| | | | interval(s) against | |
| | | | Vaccinia Virus, Elstree | |
| Parameter | | | strain, ATCC VR1549 | |
| | application | contact | temperature | With clean |
| product | conc. | time | (° C.) | conditions |
| Tween20 | 1% | 60 | 20 | 1.88 ± 0.41 |
| Ecosurf EH-9 | 1% | 60 | 20 | 2.63 ± 0.45 |
| Ecosurf SA-7 | 1% | 60 | 20 | 2.13 ± 0.25 |
| Ecosurf SA-9 | 1% | 60 | 20 | 3.13 ± 0.25 |
| DDAC | 0.015% | 60 | 20 | 4.13 ± 0.25 |
| Braunol | 0.05% | 60 | 20 | 4.00 ± 0.52 |
| povidone iodine | | | | |

| | | | | Titer (log₁₀) |
|---|---|---|---|---|
| | application | contact | temperature | with clean |
| control | conc. | time | (° C.) | conditions |
| WSH | — | 60 | 20 | 5.63 ± 0.25 |

### Results:

Thus, the virucidal test clearly—and surprisingly—demonstrates that the Ecosurf SA line, the cationic detergent DDAC as well as the disinfectant povidone iodine have a strong virucidal activity. Povidone iodine and DDAC fulfill the requirement of the DIN EN 14476:2019-10 for virucidal activity by reducing the virus titer by 4 log₁₀ grades. Therefore, these virus-deactivating substances can be used alone in order to provide the virus-inactivated biological sample. Furthermore, as shown below, also higher concentrations of these virus-deactivating substances can be used, if their inhibitory effect on the amplification reaction is counteracted. Ecosurf SA-7 and SA-9 both show strong cytotoxicity (2-3 log steps). As shown by the examples, members of the Ecosurf series, such as in particular Ecosurf SA-9, can reduce the virus titer by more than 3 log₁₀ grades at the tested concentrations. This result is also in accordance with DIN EN 14476:2019-10 for substances which have a strong cytotoxic effect and therefore require dilutions which do not allow the 4 log₁₀ grades. Furthermore, also higher concentrations could be used (see Example 1). Tween20 showed the least reduction of the virus titer at the tested concentration. As disclosed herein, it can be used at higher concentrations also in combination with other virus-deactivating substances such as SDS and/or heating to prepare a virus-inactivated biological sample that can be directly used in the amplification reaction without prior purification of the viral nucleic acids.

It was very surprising that the non-ionic surfactants Ecosurf SA-7 and Ecosurf SA-9, the cationic surfactant DDAC as well as povidone iodine fulfill the criteria for virucidal activity at these low concentrations that were shown to have no inhibitory effect on the (RT-)qPCR.

The examples in combination therefore demonstrate that the direct PCR detection of viruses is possible using the method of the invention, even after significant reduction of the virus titer fulfilling the requirement for successful virus inactivation.

### Example 4: Demonstration of Virucidal Activity for Bovine Coronavirus

The following test for virucidal activity of the different test substances were performed by Henkel (Dusseldorf) according to the European norm DIN EN 14476:2019-10. The experimental conditions were identical to those in Example 3 but with a Bovine coronavirus instead of Vaccinia virus. This demonstrates the transferability of test results for applications with severe acute respiratory syndrome coronavirus 2 (SARS-CoV-2) and other coronavirus-containing samples.

TABLE 6
Initial results of tests for virucidal activity after a residence time
of 15 minutes (t = 15) and 60 minutes (t = 60).

| | | Log₁₀ reduction factor(s) incl. | |
|---|---|---|---|
| | | 95% confidence interval(s) | |
| | | against Bovine coronavirus | |
| contact time: | conc. | 15 min | 60 min |
| Tween20 | 1% | — | — |
| Ecosurf EH-9 | 1% | — | — |
| Ecosurf SA-7 | 1% | >3.13 ± 0.25 | >3.88 ± 0.25 |
| Ecosurf SA-7 | 0.5% | >5.13 ± 0.25 | >4.88 ± 0.25 |
| Ecosurf SA-9 | 1% | >4.13 ± 0.25 | >3.88 ± 0.25 |
| Ecosurf SA-9 | 0.5% | >5.13 ± 0.25 | >4.88 ± 0.25 |
| DDAC | 0.015% | >5.13 ± 0.25 | >4.88 ± 0.25 |
| Braunol povidone | 0.05% | >6.13 ± 0.25 | >5.88 ± 0.25 |
| iodine | | | |
| WSH | — | 6.63 | — |

### Results:

The data supports the results of Example 3 and again demonstrated a virucidal activity of the different test substances also against RNA viruses, such as Bovine coronavirus. Especially the members of the Ecosurf SA line as non-ionic surfactants, the cationic surfactant DDAD as well as povidone iodine effectively inactivated the coronavirus at the tested concentrations. These results again demonstrate the virucidal activity of the tested substances in general and their applicability for the direct PCR analysis of different viruses, including coronaviruses such as SARS-CoV-2. Also other members of these classes, to which the tested substances belong, show a good virucidal activity while allowing the direct use of the obtained virus-inactivated biological sample in an amplification reaction.

### Example 5: PCR Compatibility of Virus Inactivation in the Transport Medium

As disclosed herein, the one or more virus-deactivating substances can be added to the transport medium containing the biological sample, such as a nasopharyngeal, oropharyngeal or nasal swab. The virus-deactivating substance may be directly included into the medium or it may be added thereto prior to or after addition of the collected biological sample. This advantageously results in virus-inactivation directly after sample collection and even before transportation of the patient sample to the laboratory that performs the amplification based assay for detecting the presence or absence of the target virus. After arrival, the patient sample can be handled due to the virus-inactivation under less restricted conditions. The virus-inactivated biological sample can be directly added to the amplification mixture, e.g. a PCR mixture or RT-PCT mixture, without prior purification of the comprised nucleic acids. As disclosed herein, the virus-inactivated biological sample may also be pretreated in advance of the amplification reaction, e.g. in order to support the release of the nucleic acids and/or in order to include a substance that can counteract an inhibitory effect of the virus-deactivating substance that was included in the medium, if such effect occurs to further improve the performance of the amplification reaction. Because of the virus-inactivation, the direct PCR analysis can be performed in a standard laboratory without the need of risk class 3 work benches or laboratories. This is a great advantage for high throughput testing of biological samples, in particular in situations such as a pandemic.

Commonly and widely used viral transport media such as UTM or VTM are based on Hanks Balanced Salt Solution (HBSS), which is basically a physiological sodium chloride (NaCl) solution. Therefore, a physiological sodium chloride solution (0.9%) was used to demonstrate that the virus-deactivating substances are compatible with standard transport media. Five swabs from the same SARS-CoV-2 negative donor were stored for different time periods in 0.9% NaCl solution with and without 0.0145% DDAC. The strong virucidal effect of DDAC at this concentration is demonstrated in Examples 3 and 4. Heat-inactivated SARS-CoV-2 virus particles were added. To detect any background effects of the negative donor sample a control sample containing only the heat-inactivated virus particles but no swab was included into the experiment.

After t=0 (starting point), day 1 (d1), day 2 (d2), day 6 (d6) and day 10 (d10), 8 μl of each storage medium was directly introduced without prior purification of the nucleic acids into (RT-)PCR for the detection of the N1 and N2 genes of SARS-CoV-2. The samples were pretreated using the QIAprep&amp Viral RNA UM Prep buffer (QIAGEN) and the (RT-)PCR was performed with the QIAprep&amp Viral RNA UM Master Mix (QIAGEN) for 45 cycles.

### Results:

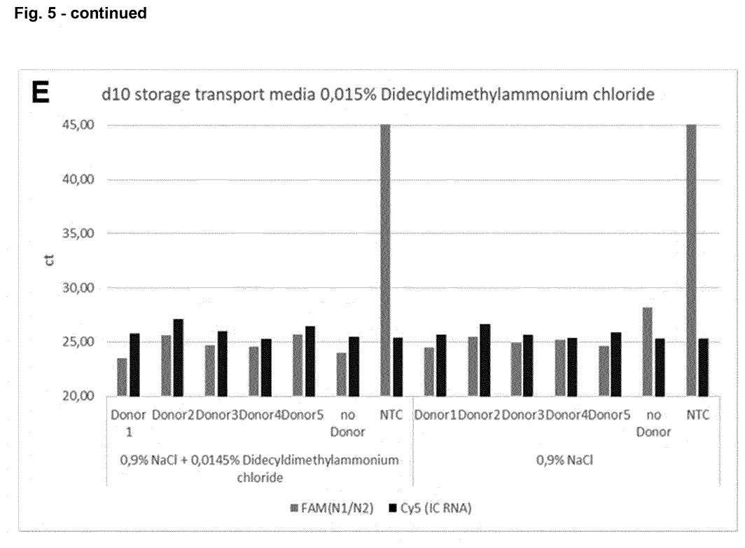

As shown in FIG. 5, there was no inhibition of the (RT-)PCR by DDAC present in the transport/storage medium. This clearly demonstrates the feasibility to inactivate the virus by using one or more virus-deactivating substances already at the earliest possible stage of the workflow, i.e. upon sample collection. This advantageously reduces the risk for people coming in contact with the collected biological sample during transport and/or analysis for the virus.

Another important aspect shown in Example 5 is the constant Ct-value. This demonstrates that the viral RNA is stable and not degraded by hydrolysis in spite of the performed virus inactivation. The so obtained virus-inactivated samples may thus also be transported and/or stored for extended periods of time, without compromising the quality of the subsequent reverse transcription amplification that is performed in order to detect the presence or absence of the target RNA virus in the collected biological sample.

### Example 6: Further Estimation of Non-Inhibiting Concentrations of Non-Ionic Surfactants in the PCR Reaction

This example is an extension of Example 1 and demonstrates the PCR compatibility of various non-ionic surfactants at different concentrations. Specifically, different polysorbates (Tween20 and Tween60), a polyoxyethylene fatty alcohol ether (Brij 58) as well as Ecosurf EH-9 and Ecosurf SA-9, were investigated in the amplification reaction at different concentrations.

The (RT-)PCR were performed with the QuantiNova Pathogen Kit (QIAGEN, Hilden) according to the manufacturer's recommendations.

### Results:

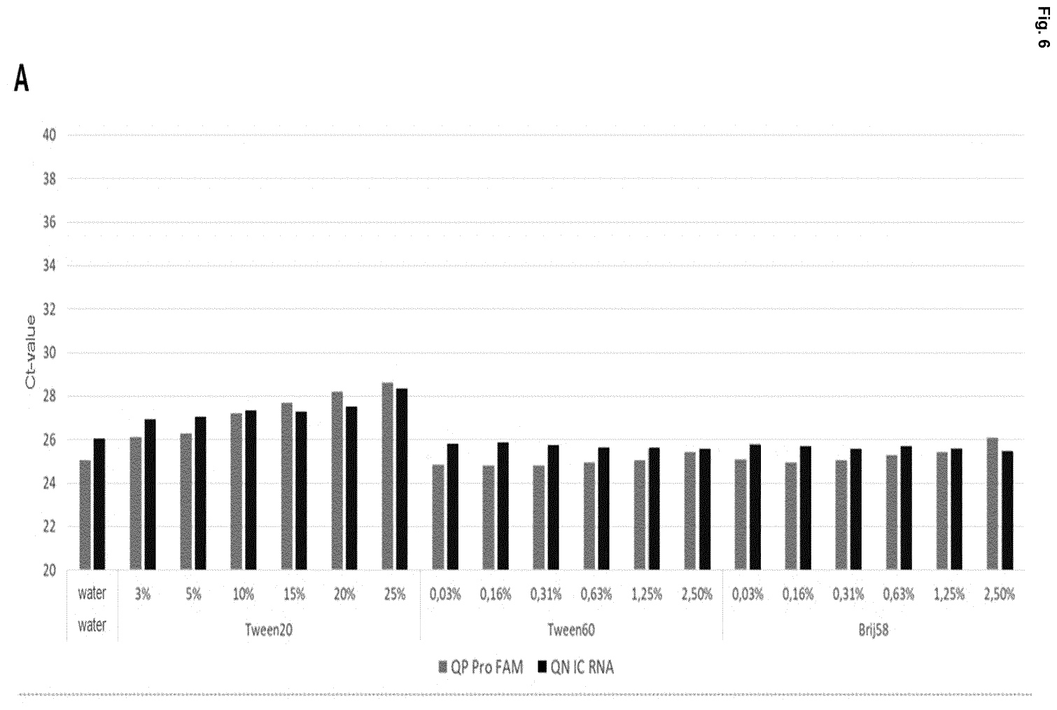

As shown in FIG. 6A only a moderate inhibition of the (RT-)PCR was observed even with very high concentrations of Tween20. The much lower concentrations of Tween60 and Brij58 tested resulted in no PCR inhibition at all. Furthermore, concentrations up to 32% of Ecosurf EH-9 and Ecosurf SA-9 did not inhibit PCR (FIG. 6B, grey bars). Ecosurf SA-9 concentrations could be increased up to 16% in RT-PCR without inhibiting the transcription and amplification reactions (FIG. 6B, black bars). Only at 32% Ecosurf SA-9 led to a significantly increased Ct-value. Moreover, concentrations of Ecosurf EH-9 of 16% or above increased the Ct-value in RT-PCR significantly. These results demonstrate that non-ionic surfactants such as Ecosurf EH-9 and Ecosurf SA-9 have an inhibitory effect on the reverse transcriptase only at very high concentrations.

This demonstrates that various non-ionic surfactants are compatible with the amplification reaction/reverse transcription amplification reaction also at high concentrations. This makes these non-ionic surfactants particularly suitable for use in combination with other virus-deactivating substances. Furthermore, as disclosed herein and demonstrated in Example 7, non-ionic surfactants can advantageously be used in order to counteract and thus neutralize the inhibiting effect of other virus-deactivating substances, e.g. anionic surfactants, such as SDS. Without being bound in theory, this advantageous counteracting or neutralizing effect may be due to mixed micelle and/or polymer pair effects (see e.g. Tomita et al, 2020). Therefore, non-ionic surfactants with virus inactivating activity can be used as virus-deactivating substance and non-ionic surfactants are also very suitable as counteracting substances as they do not have an inhibitory effect at the required concentrations.

### Example 7: PCR Inhibition by SDS and Counteracting Effect of Non-Ionic Surfactants

The anionic surfactant SDS shows a strong virucidal activity and is therefore suitable to be used as virus-deactivating substance for virus-inactivation. However, SDS has a strong inhibitory effect on the (RT-)PCR reaction. Example 7 demonstrates that this inhibitory effect of SDS on the reverse transcription amplification reaction can be effectively counteracted and thus neutralized by the addition of other detergents, in this example by different non-ionic detergents.

Template DNA and RNA in NDAB (Nucleic Acid Dilution Buffer, QIAGEN Hilden) was added to the PCR together with SDS in increasing concentrations. In addition, the same concentration series was done in the presence and absence of 3% and 5% Tween20 in the PCR, respectively. (RT-)PCR were performed with the QuantiNova Pathogen Kit (QIAGEN, Hilden) according to the manufacturer's recommendations.

### Results:

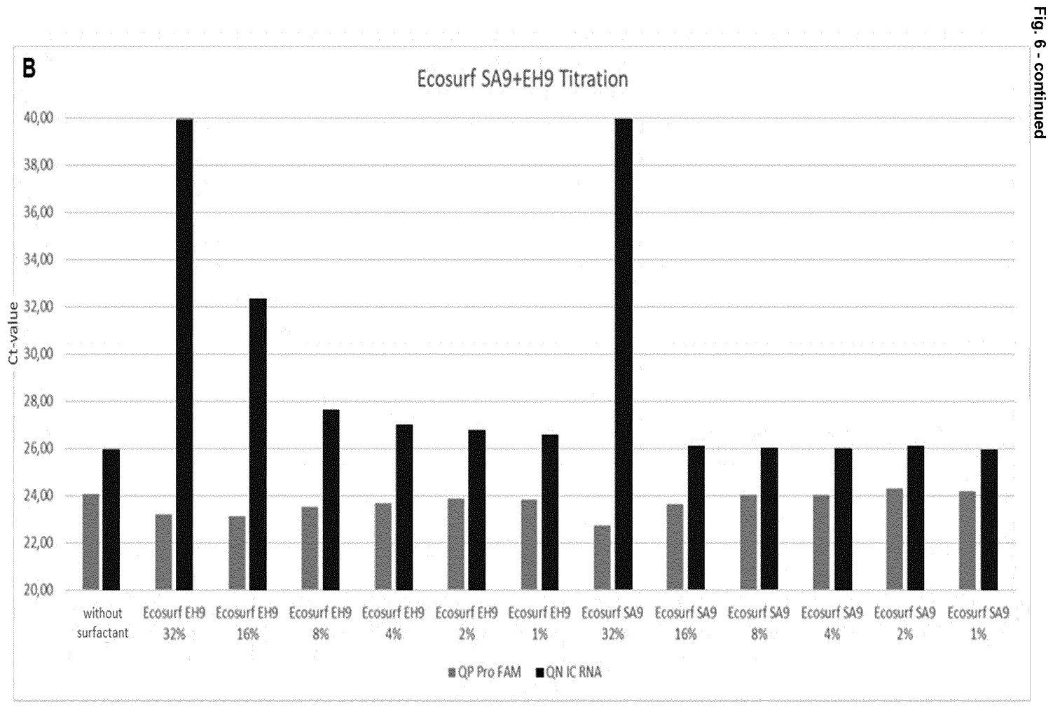

The presence of SDS in the PCR led to a significant inhibition of both, the Taq polymerase and the reverse transcriptase, as shown by increasing Ct-values starting from 0.125% SDS for the Taq polymerase and from 0.03125% for the reverse transcriptase (FIG. 7).

The addition of Tween20 reactivates the activity of the Taq polymerase even at the highest concentrations of SDS tested. The inhibitory effect on the reverse transcription (RT) was also significantly reduced depending on the used Tween20 concentration. The more Tween20 is present in the reaction, the less pronounced was the RT inhibition. Therefore, non-ionic surfactants such as polysorbates can counteract the inhibitory effects of a virus-deactivating substance, such as here SDS, on the amplification reaction and the reverse transcription. The neutralizing effect was enhanced at higher concentrations. It can be assumed that even higher concentrations of the counteracting non-ionic surfactant can result in a complete recovery of the RT activity. As demonstrated in Example 6, such non-ionic surfactants can also be used in higher concentrations in the reverse transcription reaction.

### Example 8: PCR Inhibition by SDS and DDAC and Mutual Reversion

Anionic and cationic surfactants can exhibit a strong virucidal activity and therefore, are particularly useful as virus-deactivating substances. However, as shown herein for SDS and DDAC, these surfactants can inhibit the (RT-)PCR reaction at higher concentrations when they are introduced into the reaction via the virus-inactivated biological sample from which the nucleic acids are not purified prior to performing the (RT-)PCR reaction. To investigate if SDS or DDAC can neutralize each other in a similar way as the inhibitory effect of SDS on the (RT-)PCR reaction is neutralized by a non-ionic surfactant Tween20 (see Example 7), SDS and DDAC were added in increasing concentrations to the (RT-)PCR reaction until inhibition of the Taq-polymerase and the reverse transcriptase (RT) was clearly visible. To analyze if cationic and anionic surfactants can reciprocally counteract their inhibitory effects, DDAC and SDS were added in the same ratio to the (RT)-PCR. (RT-)PCR were performed with the QIAprep&amp reaction mix (QIAGEN, Hilden) (but without the Extraction Buffer step).

### Results:

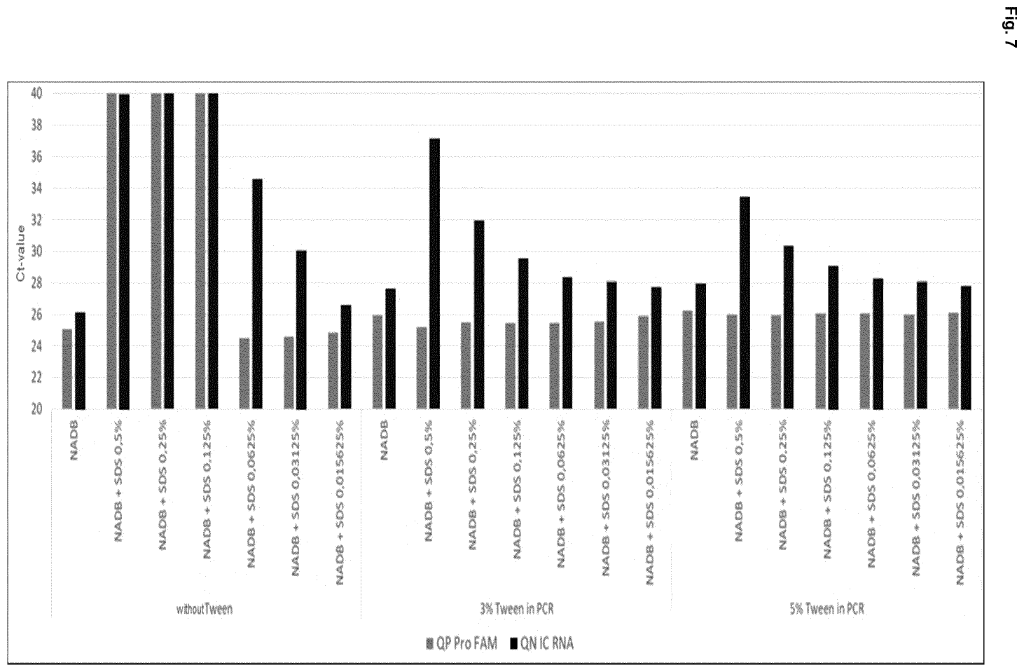

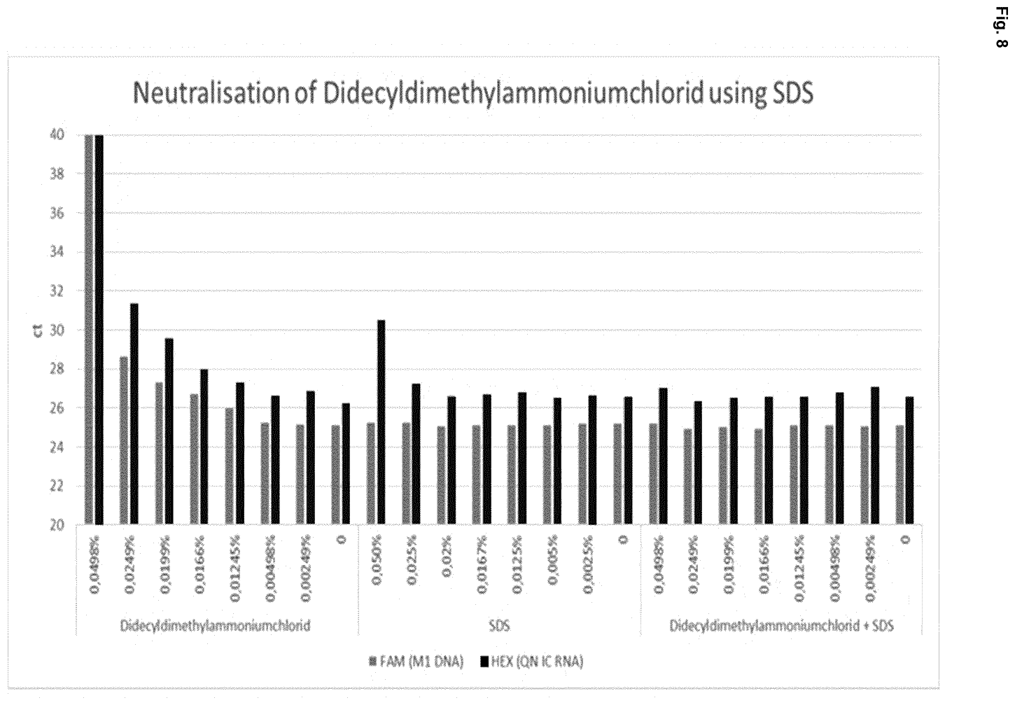

As shown in FIG. 8 the addition of SDS and DDAC led to a significant inhibition of the (RT)-PCR. Surprisingly, the addition of both virus-deactivating substances SDS and DDAC neutralizes these inhibitory effects of each other and completely restored the activity of the Taq polymerase and RT.

### Example 9: Neutralization of Povidone Iodine by a Reducing Agent

Although povidone iodine does not inhibit (RT)-PCR at concentrations sufficient to inactivate viruses as described in Example 2, there may be viruses for which a higher concentration of povidone iodine are advantageous or required for effective virus inactivation. Such higher concentrations may then lead to inhibition of the transcription and amplification reactions.

Analog to the “neutralization” of the inhibitory effect of surfactants by other surfactants as described in Examples 7 and 8, it was evaluated if povidone iodine as a redox-active-substance (oxidizing agent) can be “neutralized” by other redox-active substances (reducing agents).

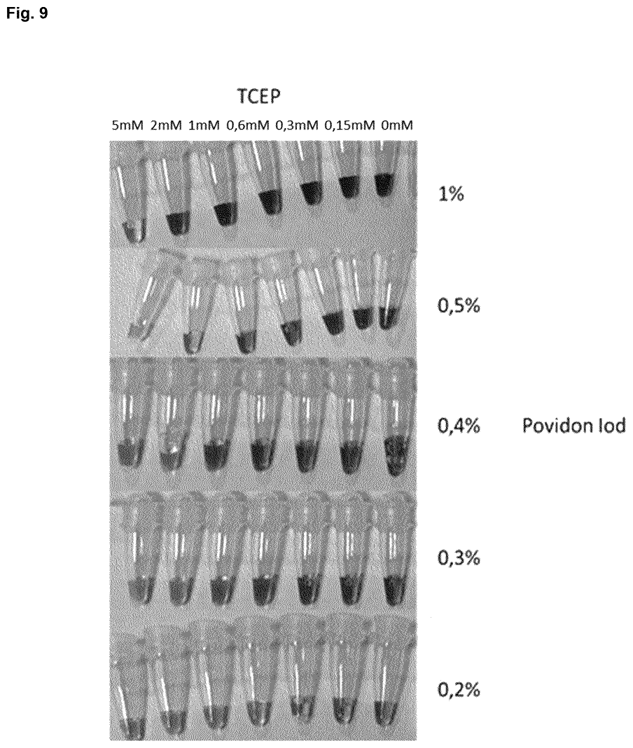

In an initial experiment the reaction of povidone iodine with TCEP was investigated. Different concentrations of povidone iodine ranging from 0.2% to 1% (see FIG. 9) were mixed with increasing amounts of TCEP. Complete “neutralization” of povidone iodine is identifiable by the disappearance of the brownish (dark) color of the iodine.

### Results:

The dark (brownish) color of povidone iodine caused by the iodine content disappears in the presence of sufficient amount of TCEP as visible in FIG. 9. This result clearly demonstrates that a reducing agent like TCEP can remove the inhibitory active oxidative component of povidone iodine and therefore should restore the full enzymatic activity in a nucleic acid amplification reaction comprising higher amounts of povidone iodine.

### Example 10: Neutralization of Povidone Iodine in Amplification

To demonstrate the “neutralizing” effect observed in Example 9, povidone iodine was added in increasing concentrations—0%/0.2%/0.3%/0.4%/0.5%/1.0%—into a 0.9% NaCl solution spiked with 2000 copies of in vitro transcribed SARS-CoV-2 genes N1 and N2, together with 200 copies of the internal RNA control (IC) and 10 ng human gDNA mimicking the background of a real sample. TCEP was added according to the different concentrations tested in Example 9 (0 mM, 0.15 mM, 0.3 mM, 0.6 mM, 1 mM, 2 mM, 5 mM).

(RT-)PCR were performed with the QIAprep&amp reaction mix (QIAGEN, Hilden) (but without the Extraction Buffer step).

### Results:

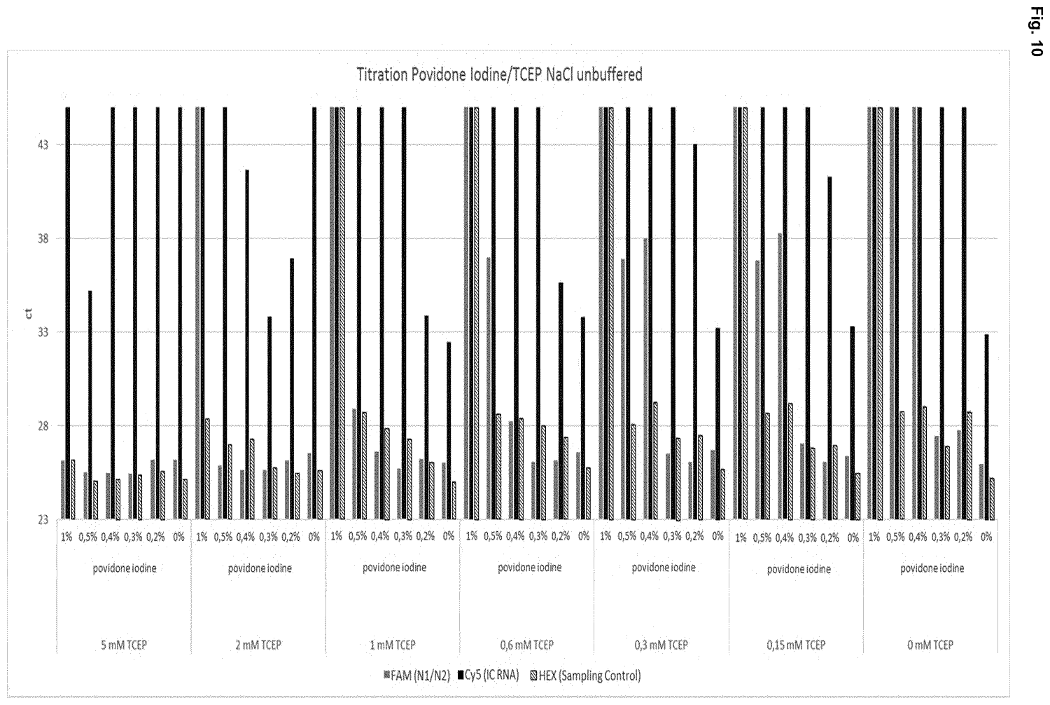

In higher concentrations povidone iodine led to a significant inhibition of the (RT)-PCR as shown in FIG. 10 (0 mM TCEP). With increasing amounts of TCEP added the inhibitory effect of povidone iodine disappears as indicated by decreasing Ct-values. For example, comparison of 0.3% povidone iodine/0 mM TCEP with 0.3% povidone iodine/1 mM TCEP results in a decrease of the Ct-value of about 2 when the N1/N2 genes of SARS-CoV-2 were analyzed.

However, in the used test setting, the addition of 2 mM or more TCEP revealed again increasing Ct-values. These results indicate that the virus-deactivating substance and the neutralizing substance need to be added in an equilibrium to effectively counteract the inhibitory effect. For instance, 5 mM TCEP neutralizes the inhibitory effect of 0.5% povidone iodine in RT-PCR. In contrast, a lower povidone iodine concentration (<0.5%) results in TCEP-induced inhibition while a higher povidone iodine concentration (>0.5%) leads to a povidone iodine-induced inhibition. Suitable concentrations for the virus-deactivating substance and the counteracting substance can thus be determined by testing different concentration series as disclosed herein.

### Example 11: Virus Inactivation of SARS-CoV-2 in Human Samples by Virus-Deactivating Substances and Direct Amplification-Based Pathogen Detection by PCR

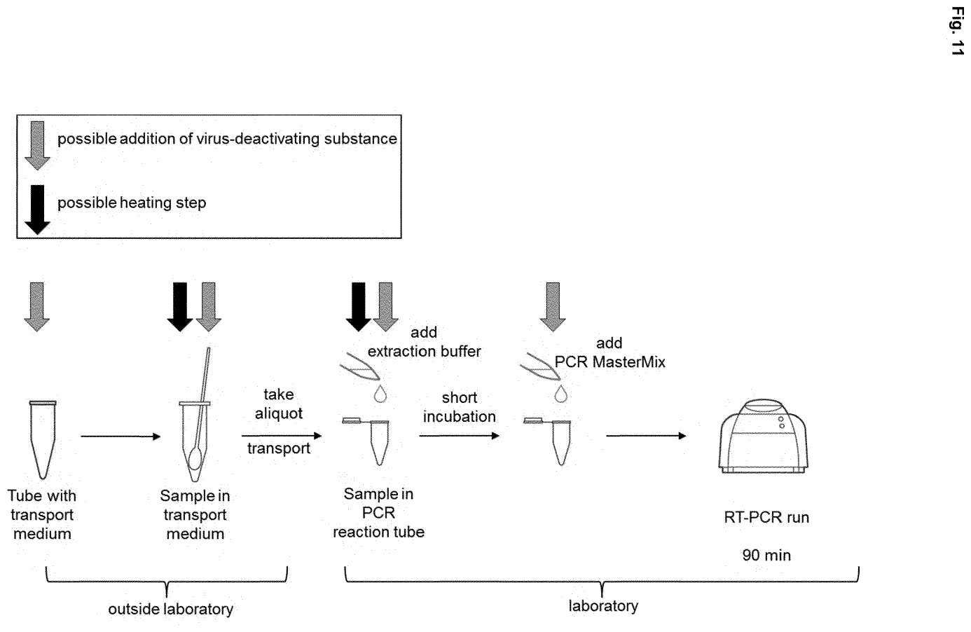

FIG. 11 illustrates exemplary workflows of the method according to the invention. These workflows can be advantageously used for the virus inactivation in human biological samples, e.g. collected with nasal, nasopharyngeal or oropharyngeal swabs, and subsequent detection of numerous pathogens by (RT-)PCR. As disclosed herein, the method according to the invention is highly effective with regard to virus inactivation, rapid, does not require prior nucleic acid purification. It allows the rapid detection of the pathogen target nucleic acids with high sensitivity also in the presence of one or more virus-deactivating substances that were used to provide the virus-inactivated sample. It is particularly suited to amplify and thus detect RNA target nucleic acids and can thus be advantageously used for the detection of RNA viruses, such as SARS-CoV-2, in human biological samples.

A virus-deactivating substance can be combined with the collected biological sample at different time points during the workflow as illustrated in FIG. 11. As disclosed herein, also two or more virus-deactivating substances can be added either at the same time or at different time points. According to one exemplary workflow, the virus-deactivating substance is included in or added to the medium. This can be a common transport medium or other saline-solution or buffer as described herein. Next, the biological sample (e.g. swab sample) is collected from the subject and placed in the medium comprising the virus-deactivating substance. It is also within the scope of the present invention to collect the biological sample (e.g. swab) as dry sample, i.e. without medium, for shipping. In this case, the dry sample is then placed in medium comprising the virus-deactivating substance when processing the biological sample. The biological sample may be agitated in the medium comprising the virus-deactivating substance (e.g. vortexing the swab containing sample), thereby providing the virus-inactivated biological sample. Subsequently, an aliquot of the virus-inactivated biological sample (comprising the biological sample, the medium and the virus-deactivating substance) is then transferred to a reaction vessel (e.g. PCR tube or well). Optionally, a pretreatment step is performed by adding an extraction composition (e.g. extraction solution) as disclosed herein to the aliquot of the virus-inactivated biological sample or the virus-inactivated biological sample is added to a reaction vessel that already contains the extraction composition. The resulting admixture may be incubated to pretreat the virus-inactivated biological sample for amplification. As disclosed herein, the incubation time may be short, thereby supporting that the method according to the present invention can be performed rapidly. Furthermore, one or more substances counteracting a potentially inhibitory effect of the virus-deactivating substance used can be included in the extraction composition. The optionally pre-treated virus-inactivated biological sample is contacted with the components necessary for performing the amplification reaction, which is a reverse transcription amplification reaction in case of RNA viral targets. While the optionally pretreated virus-inactivated biological sample may be transferred to a new reaction vessel comprising the amplification reagents, it is particularly advantageous that the method can be performed in a single reaction vessel (“one pot”). Therefore, the components necessary for performing the amplification reaction are preferably added to the same reaction vessel containing the optionally pretreated virus-inactivated biological sample. To further ease the handling and reduce pipetting errors, all components necessary for the amplification reaction may advantageously be included in a direct PCR master mix that contains besides the template all components used for the amplification reaction (including the polymerase(s), nucleotides, primers, probes, potential IC controls etc.). In one embodiment, the PCR master mix comprises at least one substance that can counteract a potential inhibitory effect of the virus-deactivating substance used and thus comprised in the (optionally pretreated) virus-inactivated biological sample. The direct PCR master mix may then be contacted with the incubated admixture and thus the optionally pretreated virus-inactivated biological sample. The so prepared amplification reaction admixture is then ready for performing the amplification reaction. As illustrated in FIG. 11, a reverse transcription amplification reaction, such as a RT-PCR, is a core embodiment of the present invention as it allows the sensitive detection of RNA viruses, such as coronaviruses. Advantageously, the reverse transcription and PCR amplification may take place in a single tube.

According to one embodiment, the biological sample (e.g. swab sample) is collected from the subject and placed in medium. As described above, this can be a common transport medium or other saline-solution or buffer as described herein. Next, the virus-deactivating substance is added to the composition comprising the biological sample and medium. It is also within the scope of the present invention to collect the biological sample (e.g. swab) as dry sample, i.e. without medium, for shipping. In this case, the dry sample is then placed in medium when processing the biological sample. The biological sample is agitated in the medium comprising the virus-deactivating substance (e.g. vortexing the swab containing sample), thereby providing the virus-inactivated biological sample. The subsequent steps may then be performed as described in the exemplary workflow above.

According to a further workflow, the biological sample (e.g. swab sample) is collected from the subject and either directly placed in medium or shipped as a dry sample and then placed in medium. The biological sample is agitated in the medium (e.g. vortexing the swab containing sample) and an aliquot of the composition comprising the medium and biological sample is then transferred to a reaction vessel (e.g. PCR tube or well). The at least virus-deactivating substance and optionally the extraction composition according to the present invention are then contacted with the composition comprising the biological sample and the medium, thereby preparing a composition that comprises the biological sample and a virus-deactivating substance (in addition the medium and optionally the extraction composition). Said composition can be incubated for efficient virus-inactivation. In another workflow, biological sample collection and pretreatment steps using an extraction composition according to the invention are performed as described above without adding a virus-deactivating substance. The virus-deactivating substance is contacted together with the components required for the amplification reaction which is then performed as described above. However, to simplify the handling of the biological samples it is preferred to introduce the virus-deactivating substance at an early stage of the workflow.

The virus-deactivating substance may be added at one or more of the above described steps.

As illustrated in FIG. 11, one or more heating steps can be included to support the lysis and/or virus inactivation. Such a heating step may be performed once the biological sample has been placed into the medium which may comprise the virus-deactivating substance. Such heating step may be performed after contacting the virus-deactivating substance with the biological sample. Furthermore, heating steps may be performed before and additionally after the addition of the virus-deactivating substance to the crude biological sample.

Preferably, no such a heating step is performed after addition of the extraction composition comprising a nuclease inhibitor, such as a proteinaceous RNase inhibitor.

The following conditions may be applied for the one or more additional heating steps to improve lysis and/or virus inactivation:

TABLE 7
Conditions for additional heating steps

| time (min) | temperature (° C.) |
|---|---|
| 30 | 56 |
| 15 | 60 |
| 10 | 65 |
| 10 | 70 |
| 5 | 90 |
| 3 | 95 |
| 2 | 98 |

1-23. (canceled)

24. A method for detecting the presence or absence of a virus in a biological sample based on amplifying at least one target nucleic acid derived from the virus without prior nucleic acid purification, comprising:
(A) providing a virus-inactivated biological sample, wherein providing such sample comprises preparing a composition comprising the biological sample and at least one virus-deactivating substance;
(B) optionally pretreating the biological sample; and
(C) subjecting at least an aliquot or all of the optionally pretreated virus-inactivated biological sample to an amplification reaction and amplifying the at least one target nucleic acid, optionally wherein a reverse transcription reaction is performed in order to reverse transcribe RNA to cDNA prior to amplification.

25. The method according to claim 24, wherein an inhibitory effect of the at least one virus-deactivating substance on the amplification reaction and/or the reverse transcription is counteracted prior to or during performing the amplification reaction in (C) by addition of at least one substance that can counteract the inhibitory effect of the at least one virus-deactivating substance, optionally wherein the at least one substance that can counteract an inhibitory effect of the at least one virus-deactivating substance is added in pretreatment step (B) and/or is included in the amplification reaction of (C).

26. The method according to claim 24, wherein at least one virus-deactivating substance is used for virus inactivation, and wherein the at least one virus-deactivating substance is a disinfectant and/or a surfactant, optionally wherein the virus-deactivating substance is selected from oxidizing agents, cationic surfactants, non-ionic surfactants, anionic surfactants and zwitterionic surfactants.

27. The method according to claim 24, wherein the virus-deactivating substance is an oxidizing agent selected from the group consisting of
(aa) iodine-releasing agents;
(bb) peroxide-based disinfectants; and
(cc) chlorine-releasing disinfectants.

28. The method according to claim 24, wherein the virus-deactivating substance is an iodophore.

29. The method according to claim 24, wherein the virus-deactivating substance is a cationic surfactant which is a quaternary ammonium salt, optionally wherein the quaternary ammonium salt has at least one of the following characteristics:
(aa) it is a tetraalkylammonium salt, wherein (i) at least one alkyl substituent has a chain length selected from C₈ to C₂₀ and (ii) two or three alkyl substituents have a chain length selected from C₁ to C₆;
(bb) it is a dialkyl dimethyl ammonium salt, wherein the chain length of the alkyl groups is selected from C₈ to C₁₆, optionally wherein the chain length of the alkyl groups is the same;
(cc) it is an alkyltrimethylammonium salt, wherein the chain length of the alkyl group is selected from C₈ to C₂₀;
(dd) it is an alkyl/aryl-quaternary ammonium salt, wherein the chain length of the alkyl group is selected from C₈ to C₂₀; and
(ee) the anion is selected from a halide and a sulfate, preferably a halide selected from chloride and bromide.

30. The method according to claim 24, wherein a cationic surfactant is used as the at least one virus-deactivating substance, and wherein the cationic surfactant is a didecyldimethylammonium halide.

31. The method according to claim 24, wherein a non-ionic surfactant is used as the at least one virus-deactivating substance, and wherein the non-ionic surfactant is an alcohol ethoxylate or an alkyl glycoside.

32. The method according to claim 31, wherein the alcohol ethoxylate is selected from the group consisting of seed oil alcohol alkoxylates and 2-ethyl hexanol ethoxylated propoxylated copolymers.

33. The method according to claim 32, wherein the virus-deactivating substance is a seed oil alcohol alkoxylate of the formula X(C₃H₆O)ₘ(C₂H₄O)ₙ, wherein X is an aromatic or aliphatic C₆ to C₁₂ alkyl group, which is linear, branched or cyclic and optionally further substituted, m is 3 or 4 and n is an integer from 4 to 9.

34. The method according to claim 24, wherein the at least one virus-deactivating substance is an anionic surfactant, optionally selected from sodium dodecyl sulfate, N-lauroyl sarcosine and caprylic acid, optionally wherein an inhibitory effect of the anionic surfactant on the amplification reaction and/or the reverse transcription is counteracted prior to or during performing the amplification reaction in (C) by addition of at least one non-ionic surfactant or a cationic surfactant.

35. The method according to claim 24, wherein the at least one virus-deactivating substance is an amine oxide-based zwitterionic surfactant.

36. The method according to claim 24, wherein one or more virus-deactivating substances selected from the group consisting
iodine-releasing agents,
quaternary ammonium salts, and
non-ionic surfactants selected from seed oil alcohol alkoxylates and 2-ethyl hexanol ethoxylated propoxylated copolymers,
are used for virus-inactivation.

37. The method according to claim 24, wherein the method has one or more of the following characteristics:
(aa) the target virus is an RNA virus, optionally an enveloped RNA virus, and wherein the amplification reaction is a reverse transcription amplification reaction;
(bb) the target virus is a coronavirus and the at least one target nucleic acid to be amplified is derived from a coronavirus;
(cc) the at least one target nucleic acid to be amplified for virus detection is derived from a severe acute respiratory syndrome-related coronavirus, optionally wherein the one or more target nucleic acids are derived from SARS-CoV-2, optionally wherein the one or more target nucleic acid sequences are selected from the SARS-CoV-2 genes N, N1, N2, RdRP, E and Orf1b;
(dd) two or more virus-deactivating substances are used for virus inactivation; and
(ee) a heating step is performed to assist virus inactivation.

38. The method according to claim 24, wherein the composition provided in (A) comprises medium that was used for collecting and/or storing the biological sample, optionally wherein the medium has one or more of the following characteristics:
(aa) the medium is a transport medium, optionally a transport medium for swab and/or smear samples;
(bb) the medium is an aqueous solution;
(cc) the medium is a saline solution suitable to keep the osmotic pressure in cells comprised in the biological sample when the medium is in contact with the biological sample;
(dd) the medium stabilizes the at least one target nucleic acid against degradation;
(ee) the medium stabilizes cells and/or viral particles comprised in the biological sample; and
(ff) the medium comprises at least one virus-deactivating substance.

39. The method according to claim 24, wherein pretreating in (B) is performed and wherein pretreating comprises:
contacting the biological sample with an extraction composition comprising
(a) at least one surfactant,
(b) at least one nuclease inhibitor, and/or
(c) at least one reducing agent,
thereby providing an admixture; and
incubating the admixture to provide a pretreated virus-inactivated biological sample.

40. The method according to claim 39, wherein the method has one or more of the following characteristics:
(aa) the virus-deactivating substance differs from the surfactant comprised in the extraction composition;
(bb) the virus-inactivated biological sample is contacted with an extraction composition comprising a surfactant that is suitable to counteract an inhibitory effect of the at least one virus-deactivating substance comprised in the virus-inactivated biological sample on the amplification reaction and/or the reverse transcription reaction performed in (C),
(cc) the extraction composition is selected from the following embodiments:
(i) the extraction composition comprises
(a) at least one non-ionic surfactant,
(b) at least one proteinaceous RNase inhibitor, and
(c) at least one reducing agent;
(ii) the extraction composition comprises
(a) at least one polyoxyethylene-based non-ionic surfactant,
(b) at least one proteinaceous RNase inhibitor, and
(c) at least one reducing agent selected from Tris(carboxyethyl)phosphine (TCEP), Dithiothreitol (DTT), N-acetyl cysteine, THPP (Tris(hydroxypropyl)phosphine) and 1-thioglycerol;
(iii) active ingredients of the extraction composition consist essentially of
(a) a non-ionic surfactant, preferably a polyoxyethylene-based non-ionic surfactant,
(b) a proteinaceous RNase inhibitor, and
(c) a reducing agent;
(iv) the extraction composition comprises
(a) at least one polysorbate,
(b) at least one proteinaceous RNase inhibitor, and
(c) Tris(carboxyethyl)phosphine (TCEP);
(v) active ingredients of the extraction composition consist essentially of
(a) at least one polysorbate,
(b) at least one proteinaceous RNase inhibitor, and
(c) Tris(carboxyethyl)phosphine (TCEP).

41. The method according to claim 24, wherein the biological sample is a respiratory biological sample and the virus is a RNA virus, and wherein the method comprises
(A) providing a virus-inactivated biological sample, wherein providing such sample comprises preparing a composition comprising the biological sample and at least one virus-deactivating substance, and wherein providing comprises contacting the biological sample with at least one virus-deactivating substance, wherein the virus-deactivating substance is selected from the group consisting of oxidizing agents such as iodophores, cationic surfactants, non-ionic surfactants, and zwitterionic surfactants, and incubating the composition to provide the virus-inactivated biological sample;
(B) optionally pretreating the biological sample, wherein optionally pretreating comprises contacting an aliquot or all of the biological sample with an extraction composition comprising
(a) at least one surfactant,
(b) at least one nuclease inhibitor, and/or
(c) at least one reducing agent,
thereby providing an admixture; and incubating the admixture to provide the pretreated virus-inactivated biological sample; and
(C) subjecting at least an aliquot or all of the optionally pretreated virus-inactivated biological sample to a reverse transcription and amplification reaction, wherein the optionally pretreated virus-inactivated biological sample is in contact with the components used for performing the reverse transcription amplification reaction thereby providing an amplification reaction admixture, wherein the prepared amplification reaction admixture comprises
(a) the optionally pretreated virus-inactivated biological sample,
(b) a DNA polymerase,
(c) a reverse transcriptase,
(d) an amplification reaction buffer comprising a Mg²⁺ source, a buffering agent and optionally further additives,
(e) nucleotides, and
(f) primers for reverse transcribing and amplifying the one or more target nucleic acids, and
performing the reverse transcription and amplification reaction to reverse transcribe and amplify at least one RNA target nucleic acid derived from the RNA virus.

42. The method according to claim 41, wherein pretreatment step (B) is performed and wherein in (C) the pretreated virus-inactivated biological sample provides at least 20% of the total reaction volume of the prepared amplification reaction admixture; and
wherein at least the steps of
contacting the biological sample with the extraction composition to prepare the admixture,
incubating the admixture, and
performing the reverse-transcription amplification reaction, are performed within the same reaction vessel, and wherein the target nucleic acid is provided by one or more, preferably two or more, target nucleic acids derived from a severe acute respiratory syndrome-related coronavirus.

43. The method according to claim 24, wherein the virus-deactivating substance reduces the virus titer of the virus-inactivated biological sample obtained in (A) compared to the biological sample by >2 log₁₀.

44. A kit for performing a method as defined in claim 24, the kit comprising:
(a) a virus-deactivating substance, wherein the virus-deactivating substance is a disinfectant and/or a surfactant, optionally wherein the virus-deactivating substance is selected from oxidizing agents, cationic surfactants, non-ionic surfactants, anionic surfactants and zwitterionic surfactants; and
one or more and preferably all of the following components:
(b) a DNA polymerase;
(c) a reverse transcriptase;
(d) an amplification reaction buffer comprising a Mg²⁺ source, a buffering agent and optionally further additives;
(e) nucleotides; and
(f) primers for amplifying the at least one target nucleic acid,
optionally wherein components (b) to (e) or (b) to (f) are comprised in a single composition, and
(g) an extraction composition comprising (a) at least one surfactant, (b) at least one nuclease inhibitor, and/or (c) at least one reducing agent.
